(19) United States
(12) Patent Application Publication
(10) Pub. No.: US 2026/0242469 A1
(43) Pub. Date: Aug. 20, 2026

(54) ANTI-CD2 ANTIBODIES FOR AMYOTROPHIC LATERAL SCLEROSIS

(71) Applicant: ZELARION MALTA LIMITED, Birkirkara (MT)

(72) Inventors: Erik BERGLUND, New York, NY (US); David BERGLUND, New York, NY (US); Alan Slade, Basking Ridge, NJ (US)

(21) Appl. No.: 19/160,969

(22) PCT Filed: Feb. 29, 2024
(86) PCT No.: PCT/EP2024/055255

§ 371 Date: Aug. 29, 2025

Related U.S. Application Data
(60) Provisional application No. 63/487,840, filed on Mar. 1, 2023.
(60) Provisional application No. 63/626,815, filed on Jan. 30, 2024.

(51) Int. Cl. C07K 16/28; A61K 39/00

(52) U.S. Cl. CPC C07K 16/2806; A61K 2039/54; A61K 2039/545; C07K 2317/24; C07K 2317/524; C07K 2317/526; C07K 2317/565

(57) ABSTRACT

Provided herein is an anti-CD2 antibody or antigen binding fragment thereof for treating and/or preventing ALS in a subject in need thereof.

### 1. CROSS-REFERENCE TO RELATED APPLICATIONS

The application is a U.S. National Stage Application under 35 U.S.C. § 371 of International Patent Application No. PCT/EP2024/055255, filed Feb. 29, 2024, which claims the benefit of U.S. Provisional Patent Application No. 63/487,840, filed Mar. 1, 2023, and U.S. Provisional Patent Application No. 63/626,815, filed Jan. 30, 2024, the content of each of which is incorporated by reference herein in its entirety.

### 2. REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14526-018-228_SEQ_LISTING.xml”, was created on Feb. 16, 2024, and is 49,151 bytes in size.

### 3. FIELD

The present application relates to anti-CD2 antibodies for treating and/or preventing amyotrophic lateral sclerosis (ALS).

### 4. BACKGROUND

ALS, also known as motor neuron disease, is characterized by neurodegeneration, leading to progressive loss of motor neurons. Neuroinflammation is an underlying contributor to amyotrophic lateral sclerosis (ALS). Typically, the degeneration of motor neurons usually begins in limb or bulbar muscles first before spreading to other body regions, and eventually ending with respiratory muscle dysfunction. Whereas a minor fraction (5-10%) of ALS is familial and several gene mutations are implicated in ALS pathogenesis, the majority (90-95%) of ALS is sporadic and the exact mechanism(s) contributing to disease progression remains unknown.

Currently, ALS remains a fatal disease as there is no cure for ALS. There are currently three drugs approved by the U.S. Food and Drug Administration (FDA) indicated for the treatment of ALS: AMX0035 (a combination of sodium phenylbutyrate and taurursodiol, also known as RELYVRIO™); edaravone (also known as RADICAVA® and RADICAVA ORS®); and riluzole (also known as RILUTEK®, TIGLUTIK™ and EXSERVAN™). However, these therapies are only capable of extending the survival of ALS patients by a few months. There is also evidence that the condition is increasing in incidence. As such, there is a need in the field to identify and develop more effective and beneficial treatments and regimens for ALS patients.

The present disclosure indicates that an anti-CD2 antibody can modulate the neuroinflammatory responses in the central nervous system (CNS) associated with motor neuron death. Provided herein is an anti-CD2 antibody (e.g., anti-CD2 Antibody 1) and methods of use thereof for treating ALS in subjects diagnosed with either sporadic or familial ALS.

### 5. SUMMARY

In one aspect, the present disclosure provides a method of treating, preventing or ameliorating at least one symptom associated with amyotrophic lateral sclerosis (ALS) in a subject in need thereof, wherein the method comprises administering to the subject an effective amount of a recombinant anti-CD2 antibody or an antigen binding fragment thereof.

In certain embodiments, the recombinant anti-CD2 antibody comprises: (a) a heavy chain variable region CDR1 comprising the amino acid sequence of SEQ ID NO: 3; (b) a heavy chain variable region CDR2 comprising the amino acid sequence of SEQ ID NO: 4; (c) a heavy chain variable region CDR3 comprising the amino acid sequence of SEQ ID NO: 5; (d) a light chain variable region CDR1 comprising the amino acid sequence of SEQ ID NO: 6; (e) a light chain variable region CDR2 comprising the amino acid sequence of SEQ ID NO: 7; and (f) a light chain variable region CDR3 comprising the amino acid sequence of SEQ ID NO: 8.

In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 1. In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 2.

In certain embodiments, the heavy chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 16. In certain embodiments, the light chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 17.

In certain embodiments, the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 9, 10, 11, 12, or 13. In certain embodiments, the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 14 or 15.

In certain embodiments, the recombinant anti-CD2 antibody comprises: (a) a heavy chain variable region CDR1 comprising the amino acid sequence of SEQ ID NO: 24; (b) a heavy chain variable region CDR2 comprising the amino acid sequence of SEQ ID NO: 25; (c) a heavy chain variable region CDR3 comprising the amino acid sequence of SEQ ID NO: 26; (d) a light chain variable region CDR1 comprising the amino acid sequence of SEQ ID NO: 27; (e) a light chain variable region CDR2 comprising the amino acid sequence of SEQ ID NO: 28; and (f) a light chain variable region CDR3 comprising the amino acid sequence of SEQ ID NO: 29.

In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 22. In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 23.

In certain embodiments, the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 30. In certain embodiments, the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 31.

In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 31, 32, 33, 34, or 35. In certain embodiments, the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 36, 37, 38, 39, or 40.

In certain embodiments, the anti-CD2 antibody or antigen binding fragment thereof comprises an additional CH2 domain. In some embodiments, the additional CH2 domain comprises an amino acid sequence that is about or at least about 80%, 85%, 90%, 95%, 98%, or 99% identical to the amino acid sequence of SEQ ID NO: 18 or 19.

In certain embodiments, the anti-CD2 antibody or antigen binding fragment thereof comprises an additional CH3 domain. In some embodiments, the additional CH3 domain comprises an amino acid sequence that is about or at least about 80%, 85%, 90%, 95%, 98%, or 99% identical to the amino acid sequence of SEQ ID NO: 20 or 21.

In certain embodiments, the anti-CD2 antibody or antigen binding fragment thereof is a humanized antibody.

In certain embodiments, the anti-CD2 antibody or antigen binding fragment thereof is an IgG1. In certain embodiments, the anti-CD2 antibody or antigen binding fragment thereof is an IgG4.

In certain embodiments, the effective amount is sufficient to: (a) increase the subject's Revised Amyotrophic Lateral Sclerosis Functional Rating Scale (ALSFRS-R) total score by at least 0.5 points; (b) maintain the subject's ALSFRS-R total score; or (c) slow the subject's disease progression rate by at least 0.5 ALSFRS-R/month, as compared to the ALSFRS-R total score obtained from the subject prior to the administering.

In certain embodiments, the effective amount is sufficient to increase life expectancy of the subject after the administering as compared to a subject having a similar diagnosis and rate of disease progression that was not administered the effective amount of a recombinant anti-CD2 antibody or an antigen binding fragment thereof. In some embodiments, the increase is an increase of about or at least about a month, 3 months, 6 months, a year, 2 years, 3 years, 4 years, 5 years, or greater than 5 years.

In certain embodiments, the effective amount is sufficient to increase the percentage of the ratio of regulatory T cells to effector T cells (Treg:Teff) in a biological sample from the subject after the administering as compared to the percentage of Treg:Teff ratio in a biological sample obtained from the subject prior to the administering. In some embodiments, the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

In certain embodiments, the effective amount is sufficient to decrease circulating T-cells in a biological sample from the subject after the administering as compared to the amount of circulating T-cells in a biological sample obtained from the subject prior to the administering. In some embodiments, the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

In certain embodiments, the effective amount is sufficient to decrease natural killer (NK)-cells in a biological sample from the subject after the administering as compared to the amount of NK-cells in a biological sample obtained from the subject prior to the administering. In some embodiments, the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

In certain embodiments, the effective amount is sufficient to increase regulatory T cells in a biological sample from the subject after the administering as compared to the amount of regulatory T cells in a biological sample obtained from the subject prior to the administering. In some embodiments, the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

In certain embodiments, a biological sample comprises the subject's blood, serum, peripheral blood mononuclear cells (PBMCs), cerebrospinal fluid (CSF), or any combination thereof.

In certain embodiments, a subject in need thereof has been diagnosed as having familial ALS or sporadic ALS. In some embodiments, a subject in need thereof has been diagnosed as having ALS within 36 months or less. In some embodiments, a subject in need thereof has been diagnosed as having ALS within 24 months or less.

In certain embodiments, the at least one symptom associated with ALS comprises dyspnea, orthopnea, muscle weakness, dysphagia, dysarthria, or a combination of two or more thereof.

In certain embodiments, the administering to the subject comprises intravenous administration, intrathecal administration, or both.

In certain embodiments, the subject is currently being treated or has been previously treated with one or more ALS treatments. In some embodiments, the one or more ALS treatments comprises riluzole, tauroursodeoxycholic acid (TUDCA), sodium phenylbutyrate, or any combination thereof.

In certain embodiments, the administering is for about or at least about 26 weeks. In certain embodiments, the administering is for about or at least about 24 weeks. In some embodiments, the administering is once every week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 3 months, once every 3.5 months, once every 4 months, once every 4.5 months, once every 5 months, once every 5.5 months, or once every 6 months.

In certain embodiments, the effective amount is sufficient to achieve a trough concentration (C0) greater than about 19 g/mL recombinant anti-CD2 antibody or an antigen binding fragment thereof in the subject. In certain embodiments, the effective amount is sufficient to achieve about 10 ng/ml recombinant anti-CD2 antibody or an antigen binding fragment thereof in the subject's CSF at a steady state. In certain embodiments, the effective amount is sufficient to achieve about 10 ng/ml recombinant anti-CD2 antibody or an antigen binding fragment thereof in the subject's CSF at a steady state for at least 30 days. In certain embodiments, the effective amount is sufficient to achieve about 10 ng/ml recombinant anti-CD2 antibody or an antigen binding fragment thereof in the subject's CSF at a steady state for at least 50 days.

In certain embodiments, the effective amount is from about 0.01 mg/kg to about 100 mg/kg and the administering is about every 4-14 weeks for a period of time sufficient to treat, prevent, or ameliorate at least one symptom associated with ALS in the subject. In certain embodiments, the effective amount is 7 mg/kg and the administering is about every 4-14 weeks for a period of time sufficient to treat, prevent, or ameliorate at least one symptom associated with ALS in the subject. In some embodiments, the administering occurs three times between the first administration and the last administration for a total of five administrations. In some embodiments, the last administration is 26 weeks after the first administration.

In certain embodiments, the effective amount is from about 0.01 mg/kg to about 100 mg/kg and the administering is about every 4-16 weeks for a period of time sufficient to treat, prevent, or ameliorate at least one symptom associated with ALS in the subject. In certain embodiments, the effective amount is 10 mg/kg and the administering is about every 4-16 weeks for a period of time sufficient to treat, prevent, or ameliorate at least one symptom associated with ALS in the subject. In some embodiments, the administering occurs two times between the first administration and the last administration for a total of four administrations. In some embodiments, the last administration is 24 weeks after the first administration.

### 6. BRIEF DESCRIPTION OF THE FIGURES

The foregoing and other objects, features, and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1 shows an ALS study overview using an anti-CD2 antibody (e.g., anti-CD2 Antibody 1) or antigen binding fragment thereof of the present disclosure.

FIG. 2 shows the schedule of assessments using an anti-CD2 antibody (e.g., anti-CD2 Antibody 1) or antigen binding fragment thereof of the present disclosure for treatment of ALS. “X” denotes clinic visit; “V” denotes Visit; “D” denotes Day; “W” denotes Week; and “EOS” denotes end of study or early termination. Footnote “a” indicates that vital signs include blood pressure, pulse, temperature, respiratory rate and weight; height will be collected at the first visit only. Footnote “b” indicates that infectious disease screening for exclusionary infections of HIV, HBV, HCV, SARS-Cov2 and TB. Footnote “c” indicates that hematology includes hemoglobin, hematocrit, red blood cell (RBC), platelet count, white blood cells (WBC) with differential count (% and abs). Footnote “d” indicates that chemistry includes sodium, potassium, chloride, urea, bicarbonate, creatine, glucose, ALT, ALP, alkaline phosphatase, bilirubin, and CRP. Footnote “e” indicates that female participants of reproductive capacity will be monitored for pregnancy and must have a negative serum pregnancy test prior to Day 1. Footnote “f” indicates that pre-medication must precede first of CD2 Ab 1 for all subjects.

FIG. 3 shows representative oligosaccharides suitable for use in production of an anti-CD2 antibody or antigen binding fragment thereof of the present disclosure.

FIG. 4 shows an ALS study overview using an anti-CD2 antibody (e.g., anti-CD2 Antibody 1) or antigen binding fragment thereof of the present disclosure.

FIG. 5 shows the schedule of assessments using an anti-CD2 antibody (e.g., anti-CD2 Antibody 1) or antigen binding fragment thereof of the present disclosure for treatment of ALS. “X” denotes clinic visit; “V” denotes Visit; “D” denotes Day; “W” denotes Week; and “EOS” denotes end of study or early termination. Footnote “a” indicates that vital signs include blood pressure, pulse, temperature and weight; height will be collected at the first visit only. Footnote “b” indicates that infectious disease screening for HIV, HBV, HCV, EBV, CMV and TB; SARS-COV2 testing per local practice. Footnote “c” indicates that hematology includes hemoglobin, hematocrit, red blood cell (RBC), platelet count, white blood cells (WBC) with differential count (% and abs). Footnote “d” indicates that chemistry includes sodium, potassium, chloride, urea, bicarbonate, creatine, glucose, ALT, AST, alkaline phosphatase and total bilirubin. Footnote “e” indicates that female participants of reproductive capacity will be monitored for pregnancy and must have a negative serum pregnancy test prior to Day 1. Footnote “f” indicates that CSF sampling at screening (Visit 1) should only be performed when the patient has met all inclusion/exclusion criteria. Footnote “g” indicates that Day 0 PK samples will be collected prior to and at 2 (+/−10 min) and 6 hours (+/−30 min) after start of infusion. At visits up to and including Week 24, one PK sample will be collected pre-dose. Footnote “h” indicates that pre-medication must precede the first administration of siplizumab, subsequent premedication at the investigator's discretion. When anti-CD2 Antibody 1 is being administered, a PK sample should be taken prior to infusion; at non-dosing visits, PK sample can be collected at any time.

### 7. DETAILED DESCRIPTION

Provided herein is an anti-CD2 antibody or antigen binding fragment thereof and methods of use thereof for treating, preventing or ameliorating at least one symptom associated with amyotrophic lateral sclerosis (ALS) in a subject in need thereof. The anti-CD2 antibody or antigen binding fragment thereof described herein can be administered to a subject for a fixed amount of time or for a period of time sufficient to treat, prevent, or ameliorate at least one symptom associated with ALS. In some embodiments, the anti-CD2 antibody or antigen binding fragment thereof described herein is administered to a subject to treat or prevent progression of ALS.

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. In particular, unless otherwise noted the terms mean within plus or minus 10% of a given value or range. In instances where an integer is required, the terms mean within plus or minus 10% of a given value or range, rounded either up or down to the nearest integer.

### 7.1 Methods of Treating ALS

Provided herein is a method to treat a patient with ALS wherein the method comprises administering to the patient in need of an anti-CD2 antibody or antigen binding fragment thereof, such as the antibodies or antigen binding fragments thereof described in Section 7.1.1. Selection of patients for treatment with the methods provided herein is described in Section 7.1.2. Dosing regimens for administering the anti-CD2 antibody or antigen binding fragment thereof in the methods provided herein are described in Section 7.1.3 below. The methods provided herein for selecting the patients, determining the outcome of these methods, and/or serving as criteria in any way for these methods are described in Section 7.3 below. The therapeutic outcome of the methods provided herein is described in Section 7.2 below. Therefore, a person skilled in the art would understand that the methods provided herein include all permutations, including modifications, and combinations of the anti-CD2 antibodies or antigen binding fragments thereof, patients, dosing regimens, diagnostic and ALS staging criteria, biomarkers, and therapeutic outcomes as described above and below.

### 7.1.1 Antibodies for Use with the Methods of Treating ALS

The anti-CD2 antibody or antigen binding fragment thereof of the present disclosure is described in Section 7.1.1.1. Methods for generating the anti-CD2 antibody or antigen binding fragment thereof of the present disclosure are described in Section 7.1.1.2. Pharmaceutical compositions and kits of the anti-CD2 antibody or antigen binding fragment thereof of the present disclosure are described in Section 7.1.1.3 and Section 7.1.1.4, respectively.

### 7.1.1.1 Anti-CD2 Antibody or Antigen Binding Fragment Thereof

Provided herein is a method of treating ALS in a subject in need thereof with an anti-CD2 antibody or antigen binding fragment thereof of the present disclosure. In some embodiments, the methods described herein include administering an anti-CD2 antibody or antigen binding fragment thereof to a subject (e.g., a juvenile or adult subject diagnosed with or suspected of having ALS).

As used herein, an antibody refers to an immunoglobulin including IgG, IgM, IgE, IgA, and IgD. The antibody described herein can be a monoclonal antibody or a polyclonal antibody. In some embodiments the antibody can be a chimeric antibody. In some embodiments, the antibody can be a humanized antibody. In a specific embodiment, the antibody is a recombinant antibody. In a specific embodiment, the antibody is a humanized antibody. In some embodiments, an anti-CD2 antibody or antigen binding fragment thereof described herein is an IgG. In some embodiments, the IgG can be an IgG1, IgG2, IgG3, or an IgG4. In specific embodiments, an anti-CD2 antibody or antigen binding fragment thereof described herein is an IgG1, IgG2, or an IgG4. As described herein, an anti-CD2 antibody or antigen binding fragment thereof described herein can be an antigen binding fragment. In certain embodiments, an antigen binding fragment is, or can comprise of, Fab, F(ab′) 2, scFv (VH fused to a VL), or sdAb.

As provided herein, the present disclosure relates, in part, to methods of treating ALS in a subject in need thereof with an anti-CD2 antibody (e.g., an anti-CD2 antibody described in WO 2021/259927) or an antigen binding fragment thereof. In some embodiments, an anti-CD2 antibody for use with the present methods can be anti-CD2 Antibody 1. As used herein, the term “anti-CD2 Antibody 1” refers to any antibody that comprises one or more of the CDRs and/or one or more of the VH and VL regions set forth in Table 1. In some embodiments, an anti-CD2 antibody or antigen binding fragment thereof for use with the methods and compositions disclosed herein has the CDR sequences of a rat anti-CD2 monoclonal antibody BTI-322 (see, e.g., WO 02/098370). In some embodiments, an anti-CD2 antibody or antigen binding fragment thereof can be a humanized IgG1 version of BTI-322 (siplizumab; previously known as MEDI-507). In some embodiments, an anti-CD2 antibody or antigen binding fragment thereof is a Fc-silent anti-CD2 antibody or an antigen binding fragment thereof. In some embodiments, an anti-CD2 antibody or antigen binding fragment thereof is BTI-322 or an antigen binding fragment thereof. In some embodiments, an anti-CD2 antibody or antigen binding fragment thereof is CB.219 (see, e.g., Erben U, et al. Clin Immunol. 2015 March; 157 (1): 16-25) or an antigen binding fragment thereof. In some embodiments, an anti-CD2 antibody or antigen binding fragment thereof is LO-CD2b (see, e.g., WO/2000/078814) or an antigen binding fragment thereof. In some embodiments, an anti-CD2 antibody or antigen binding fragment thereof is siplizumab or an antigen binding fragment thereof. In some embodiments, the methods described herein include administering at least one, at least two, at least three, or more than at least three anti-CD2 antibody or antigen binding fragment thereof. In some embodiments, the methods described herein include administering one or more than one anti-CD2 antibody or antigen binding fragment thereof. In certain embodiments, the methods described herein include administering an anti-CD2 antibody or antigen binding fragment thereof in combination with another agent to a subject. In certain embodiments, the methods described herein include administering an anti-CD2 antibody or antigen binding fragment thereof prior to, at the same time, and/or after another agent to a subject. In certain embodiments, the methods described herein include administering an anti-CD2 antibody or antigen binding fragment thereof as monotherapy (e.g., for treating ALS). In certain embodiments, the methods described herein consist essentially of administering an anti-CD2 antibody or antigen binding fragment thereof to a subject (e.g., for treating ALS). In some embodiments, the methods described herein comprise administering an anti-CD2 antibody or antigen binding fragment thereof as the only antibody to the subject (e.g., for treating ALS).

An antibody as described herein can be comprised of two heavy and two light chains connected by a disulfide bond. Each heavy chain can comprise a variable region (VH) and a constant region. Each light chain can comprise a variable region (VL) and a constant region. The variable region of both the heavy and the light chain dictates the binding of the antibody to the antigen. The complementarity determining regions (CDRs) are variable loops on the variable regions of the heavy and light chain. There are three CDRs on each heavy chain and three CDRs on each light chain. In certain embodiments, the antibody as described herein binds to CD2.

In certain embodiments, administration of an anti-CD2 antibody or antigen binding fragment thereof described herein does not result in target cell depletion. In certain embodiments, an anti-CD2 antibody or antigen binding fragment thereof described herein exhibits immunomodulatory activity. In a specific embodiment, the ability of an anti-CD2 antibody or antigen binding fragment thereof described herein to refrain from target cell depletion while retaining immunomodulatory activity is accomplished by eliminating glycosylation of Fc region.

In some embodiments, an anti-CD2 antibody (e.g., anti-CD2 Antibody 1) or antigen binding fragment thereof as described herein binds specifically to CD2 (also called T11, SRBC (sheep red blood cell receptor), and LFA-2). In a specific embodiment, an anti-CD2 antibody or antigen binding fragment thereof described herein binds to human CD2 (GenBank Accession No. NM_001328609.1 (isoform 1); NM_001767.5 (isoform 2)). In some embodiments, an anti-CD2 antibody (e.g., anti-CD2 Antibody 1) or antigen binding fragment thereof binds to a unique epitope on human CD2, distinct from the CD58 binding site, with high affinity (kd-5 nM), inhibiting co-stimulation and T-cell activation. As used herein, an epitope is the region of the antigen to which the antibody or the antigen binding fragment binds. In some embodiments, the epitope can be linear. In some embodiments, the epitope can be conformational. In some embodiments, the epitope can be formed by contiguous amino acids. In some embodiments, the epitope can be formed by noncontiguous amino acids. In certain embodiments, the anti-CD2 antibody or antigen binding fragment thereof described herein binds to an epitope on CD2. In certain embodiments, the anti-CD2 antibody or antigen binding fragment thereof provided herein has an IC50 value of about 0.5 nM, about 0.6 nM, about 0.7 nM, about 0.8 nM, about 0.9 nM, about 1.0 nM, about 1.1 nM, about 1.2 nM, about 1.3 nM, about 1.4 nM, about 1.5 nM, between about 0.5 nM and about 0.8 nM, between about 0.6 nM and about 0.9 nM, between about 0.7 nM and about 1.0 nM, between about 0.8 nM and about 1.1 nM, between about 0.9 nM and about 1.2 nM, between about 1.0 nM and about 1.3 nM, between about 1.1 nM and about 1.4 nM, or between about 1.2 nM and about 1.5 nM.

In certain embodiments, the anti-CD2 antibody or antigen binding fragment thereof provided herein has an IC50 value of at least about 0.5 nM, about 0.6 nM, about 0.7 nM, about 0.8 nM, about 0.9 nM, about 1.0 nM, about 1.1 nM, about 1.2 nM, about 1.3 nM, about 1.4 nM, about 1.5 nM, between about 0.5 nM and about 0.8 nM, between about 0.6 nM and about 0.9 nM, between about 0.7 nM and about 1.0 nM, between about 0.8 nM and about 1.1 nM, between about 0.9 nM and about 1.2 nM, between about 1.0 nM and about 1.3 nM, between about 1.1 nM and about 1.4 nM, or between about 1.2 nM and about 1.5 nM.

In certain embodiments, the anti-CD2 antibody or antigen binding fragment thereof provided herein has an IC50 value of at most about 0.5 nM, about 0.6 nM, about 0.7 nM, about 0.8 nM, about 0.9 nM, about 1.0 nM, about 1.1 nM, about 1.2 nM, about 1.3 nM, about 1.4 nM, about 1.5 nM, between about 0.5 nM and about 0.8 nM, between about 0.6 nM and about 0.9 nM, between about 0.7 nM and about 1.0 nM, between about 0.8 nM and about 1.1 nM, between about 0.9 nM and about 1.2 nM, between about 1.0 nM and about 1.3 nM, between about 1.1 nM and about 1.4 nM, or between about 1.2 nM and about 1.5 nM.

In certain embodiments, the sequence of the VH region of the anti-CD2 antibody (e.g., anti-CD2 Antibody 1) or antigen binding fragment thereof described herein is 80%, 85%, 90%, 91%, 92%, 93%, 94%, 95%, 96%, 97%, 98%, 99%, or 100% identical to SEQ ID NO: 1. In certain embodiments, the sequence of the VL region of the anti-CD2 antibody or antigen binding fragment thereof described herein is 80%, 85%, 90%, 91%, 92%, 93%, 94%, 95%, 96%, 97%, 98%, 99%, or 100% identical to SEQ ID NO: 2. In certain embodiments, the sequence of the VH CDR1 of the anti-CD2 antibody or antigen binding fragment thereof described herein is, or comprises, SEQ ID NO: 3. In certain embodiments, the sequence of the VH CDR2 of the anti-CD2 antibody or antigen binding fragment thereof described herein is, or comprises, SEQ ID NO: 4. In certain embodiments, the sequence of the VH CDR3 of the anti-CD2 antibody or antigen binding fragment thereof described herein is, or comprises, SEQ ID NO: 5. In certain embodiments, the sequence of the VL CDR1 of the anti-CD2 antibody or antigen binding fragment thereof described herein is, or comprises, SEQ ID NO: 6. In certain embodiments, the sequence of the VL CDR2 of the anti-CD2 antibody or antigen binding fragment thereof described herein is, or comprises, SEQ ID NO: 7. In certain embodiments, the sequence of the VL CDR3 of the anti-CD2 antibody or antigen binding fragment thereof described herein is, or comprises, SEQ ID NO: 8. In certain embodiments, the sequence of the VH CDR1 of the anti-CD2 antibody or antigen binding fragment thereof described herein comprises an amino acid sequence consisting of SEQ ID NO: 3. In certain embodiments, the sequence of the VH CDR2 of the anti-CD2 antibody or antigen binding fragment thereof described herein comprises an amino acid sequence consisting of SEQ ID NO: 4. In certain embodiments, the sequence of the VH CDR3 of the anti-CD2 antibody or antigen binding fragment thereof described herein comprises an amino acid sequence consisting of SEQ ID NO: 5. In certain embodiments, the sequence of the VL CDR1 of the anti-CD2 antibody or antigen binding fragment thereof described herein comprises an amino acid sequence consisting of SEQ ID NO: 6. In certain embodiments, the sequence of the VL CDR2 of the anti-CD2 antibody or antigen binding fragment thereof described herein comprises an amino acid sequence consisting of SEQ ID NO: 7. In certain embodiments, the sequence of the VL CDR3 of the anti-CD2 antibody or antigen binding fragment thereof described herein comprises an amino acid sequence consisting of SEQ ID NO: 8. These sequences are shown in Table 1.

In certain embodiments, the anti-CD2 antibody (e.g., anti-CD2 Antibody 1) or antigen binding fragment thereof for use with the methods provided herein has a heavy chain variable region comprising VH CDRs of SEQ ID NOS: 3-5, respectively, and a VL of SEQ ID NO: 2. In certain embodiments, the anti-CD2 antibody or antigen binding fragment thereof for use with the methods provided herein has a light chain variable region comprising VL CDRs of SEQ ID NOS: 6-8, respectively, and a VH of SEQ ID NO: 1.

In certain embodiments, the anti-CD2 antibody (e.g., anti-CD2 Antibody 1) or antigen binding fragment thereof for use with the methods provided herein has a heavy chain variable region CDR1 of SEQ ID NO: 3; a heavy chain variable region CDR2 of SEQ ID NO: 4; a heavy chain variable region CDR3 of SEQ ID NO: 5; a light chain variable region CDR1 of SEQ ID NO: 6; a light chain variable region CDR2 of SEQ ID NO: 7; and a light chain variable region CDR3 of SEQ ID NO: 8. In certain embodiments, the anti-CD2 antibody or antigen binding fragment thereof for use with the methods provided herein has a heavy chain variable region CDR1 comprising a sequence that is at least about or about 75%, 80%, 85%, 90%, 95%, 96%, 97%, 98%, 99%, or 100% identical to SEQ ID NO: 3; a heavy chain variable region CDR2 comprising a sequence that is at least about or about 75%, 80%, 85%, 90%, 95%, 96%, 97%, 98%, 99%, or 100% identical to SEQ ID NO: 4; a heavy chain variable region CDR3 comprising a sequence that is at least about or about 75%, 80%, 85%, 90%, 95%, 96%, 97%, 98%, 99%, or 100% identical to SEQ ID NO: 5; a light chain variable region CDR1 comprising a sequence that is at least about or about 75%, 80%, 85%, 90%, 95%, 96%, 97%, 98%, 99%, or 100% identical to SEQ ID NO: 6; a light chain variable region CDR2 comprising a sequence that is at least about or about 75%, 80%, 85%, 90%, 95%, 96%, 97%, 98%, 99%, or 100% identical to SEQ ID NO: 7; and/or a light chain variable region CDR3 comprising a sequence that is at least about or about 75%, 80%, 85%, 90%, 95%, 96%, 97%, 98%, 99%, or 100% identical to SEQ ID NO: 8.

In certain embodiments, the anti-CD2 antibody (e.g., anti-CD2 Antibody 1) or antigen binding fragment thereof for use with the methods provided herein has a heavy chain variable region CDR1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 3; a heavy chain variable region CDR2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 4; a heavy chain variable region CDR3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 5; a light chain variable region CDR1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 6; a light chain variable region CDR2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 7; and/or a light chain variable region CDR3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 8.

In some embodiments, an amino acid substitution is a conservative substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

In certain embodiments, an anti-CD2 antibody (e.g., anti-CD2 Antibody 1) or antigen binding fragment thereof for use with the present methods and compositions comprises 1, 2, or 3 of the heavy chain CDRs of humanized anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of rhesusized anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of deglycosylated rhesusized anti-CD2 antibody or antigen binding fragment thereof. In certain embodiments, an anti-CD2 antibody or antigen binding fragment thereof for use with the present methods and compositions comprises 1, 2, or 3 of the heavy chain CDRs of deglycosylated anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of a ligand blocking anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of a deglycosylated ligand blocking anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of a Fc silent ligand blocking anti-CD2 antibody or an antigen binding fragment thereof. In certain embodiments, an anti-CD2 antibody or antigen binding fragment thereof for use with the present methods and compositions comprises 1, 2, or 3 of the heavy chain CDRs of a humanized ligand blocking anti-CD2 antibody or an antigen binding fragment thereof.

In certain embodiments, an anti-CD2 antibody (e.g., anti-CD2 Antibody 1) or antigen binding fragment thereof for use with the present methods and compositions comprises 1, 2, or 3 of the heavy chain CDRs set forth in Table 1. In certain embodiments, an anti-CD2 antibody or antigen binding fragment thereof for use with the present methods and compositions comprises 1, 2, or 3 of the light chain CDRs of set forth in Table 1. In certain embodiments, an anti-CD2 antibody or antigen binding fragment thereof for use with the methods described herein comprises 1, 2, 3, 4, 5, or all 6 of the CDRs set forth in Table 1. In certain embodiments, 1, 2, 3, 4, 5, and/or all 6 of the CDRs set forth in Table 1 can have 1, 2, 3, 4, 5, 6, 7, 8, 9, or 10 amino acid substitutions. In a more specific embodiment, such an amino acid substitution is a conservative amino acid substitution.

TABLE 1
Sequences of anti-CD2 Antibody 1

| Description | Sequence |
|---|---|
| VH region | QVQLVQSGAEVQRPGASVKVSCKASGYIF |
| (SEQ ID NO: 1) | TEYYMYWVRQAPGQGLELVGRIDPEDGSI |
| | DYVEKFKKKVTLTADTSSSTAYMELSSLT |
| | SDDTAVYYCARGKFNYRFAYWGQGTLVTV |
| | SS |
| VL region | DVVMTQSPPSLLVTLGQPASISCRSSQSL |
| (SEQ ID NO: 2) | LHSSGNTYLNWLLQRPGQSPQPLIYLVSK |
| | LESGVPDRFSGSGSGTDFTLKISGVEAED |
| | VGVYYCMQFTHYPYTFGQGTKLEIK |
| VH CDR1 | EYYMY |
| (SEQ ID NO: 3) | |
| VH CDR2 | RIDPEDGSIDYVEKFKK |
| (SEQ ID NO: 4) | |
| VH CDR3 | GKFNYRFAY |
| (SEQ ID NO: 5) | |
| VL CDR1 | RSSQSLLHSSGNTYLN |
| (SEQ ID NO: 6) | |
| VL CDR2 | LVSKLES |
| (SEQ ID NO: 7) | |
| VL CDR3 | MQFTHYPYT |
| (SEQ ID NO: 8) | |

In certain embodiments, the DNA sequence of the anti-CD2 antibody (e.g., anti-CD2 Antibody 1) or antigen binding fragment thereof can be modified to optimize the product yield during manufacture. The sequence optimization can increase product yield while not impacting product quality secretion of the molecule during production since the amino acid sequence generated can be the same as described in Table 1. The optimized DNA sequence for the anti-CD2 antibody or antigen binding fragment thereof heavy chain and light chain are presented in Table 2.

TABLE 2
Optimized sequences encoding the anti-CD2 Antibody 1

| Description | Sequence |
|---|---|
| Optimized Heavy | CAAGTGCAGCTGGTGCAGAGCGGAGCTGAGGTGCAGAG |
| chain sequence | ACCCGGCGCCAGCGTCAAGGTGAGCTGTAAGGCCAGCG |
| (SEQ ID NO: 16) | GCTACATCTTCACAGAATACTACATGTACTGGGTGAGGC |
| | AAGCCCCCGGCCAAGGACTGGAGCTGGTGGGCAGAATC |
| | GATCCAGAGGATGGCAGCATCGACTACGTGGAGAAGTT |
| | CAAGAAGAAGGTGACTCTGACAGCCGACACAAGCAGCA |
| | GCACTGCTTACATGGAGCTGAGCTCTCTGACTAGCGATG |
| | ACACTGCCGTGTACTACTGTGCTAGGGGCAAGTTCAACT |
| | ATAGGTTCGCCTACTGGGGCCAAGGCACTCTGGTGACAG |
| | TCAGCAGCGCTAGCACCAAGGGCCCATCGGTCTTCCCCC |
| | TGGCACCCTCCTCCAAGAGCACCTCTGGGGGCACAGCGG |
| | CCCTGGGCTGCCTGGTCAAGGACTACTTCCCCGAACCGG |
| | TGACGGTGTCGTGGAACTCAGGCGCCCTGACCAGCGGCG |
| | TGCACACCTTCCCGGCCGTCCTACAGTCCTCAGGACTCT |
| | ACTCCCTCAGCAGCGTGGTGACCGTGCCCTCCAGCAGCT |
| | TGGGCACCCAGACCTACATCTGCAACGTGAATCACAAGC |
| | CCAGCAACACCAAGGTGGACAAGAAGGTTGAGCCCAAA |
| | TCTTGTGACAAAACTCACACATGCCCACCGTGCCCAGCA |
| | CCTGAACTCCTGGGGGGACCGTCAGTCTTCCTCTTCCCCC |
| | CAAAACCCAAGGACACCCTCATGATCTCCCGGACCCCTG |
| | AGGTCACATGCGTGGTGGTGGACGTGAGCCACGAAGAC |
| | CCTGAGGTCAAGTTCAACTGGTACGTGGACGGCGTGGAG |
| | GTGCATAATGCCAAGACAAAGCCGCGGGAGGAGCAGTA |
| | CAACAGCACGTACCGTGTGGTCAGCGTCCTCACCGTCCT |
| | GCACCAGGACTGGCTGAATGGCAAGGAGTACAAGTGCA |
| | AGGTCTCCAACAAAGCCCTCCCAGCCCCCATCGAGAAAA |
| | CCATCTCCAAAGCCAAAGGGCAGCCCCGAGAACCACAG |
| | GTGTACACCCTGCCCCCATCCCGGGACGAGCTGACCAAG |
| | AACCAGGTCAGCCTGACCTGCCTGGTCAAAGGCTTCTAT |
| | CCCAGCGACATCGCCGTGGAGTGGGAGAGCAATGGGCA |
| | GCCGGAGAACAACTACAAGACCACGCCTCCCGTGCTGG |
| | ACTCCGACGGCTCCTTCTTCCTCTACAGCAAGCTCACCGT |
| | GGACAAGAGCAGGTGGCAGCAGGGGAACGTCTTCTCAT |
| | GCTCCGTGATGCATGAGGCTCTGCACAACCACTACACGC |
| | AGAAGAGCCTCTCCCTGTCTCCGGGTAAATGATGA |
| Optimized Light | GACGTGGTGATGACTCAGAGCCCTCCTTCTCTGCTGGTG |
| chain sequence | ACTCTGGGCCAGCCAGCCAGCATCAGCTGTAGGAGCAG |
| (SEQ ID NO: 17) | CCAGTCTCTGCTGCACTCCAGCGGCAACACTTATCTGAA |
| | CTGGCTGCTGCAGAGACCCGGCCAGAGCCCTCAGCCTCT |
| | GATCTACCTCGTGAGCAAGCTGGAGAGCGGCGTGCCAG |
| | ATAGGTTTAGCGGCAGCGGAAGCGGCACTGACTTCACTC |
| | TGAAGATCAGCGGCGTGGAAGCTGAGGATGTGGGCGTC |
| | TACTACTGCATGCAGTTCACACACTACCCATACACTTTC |
| | GGCCAAGGCACAAAGCTGGAAATCAAGCGTACGGTGGC |
| | TGCACCATCTGTCTTCATCTTCCCGCCATCTGATGAGCAG |
| | TTGAAATCTGGAACTGCCTCTGTTGTGTGCCTGCTGAAT |
| | AACTTCTATCCCAGAGAGGCCAAAGTACAGTGGAAGGT |
| | GGATAACGCCCTCCAATCGGGTAACTCCCAGGAGAGTGT |
| | CACAGAGCAGGACAGCAAGGACAGCACCTACAGCCTCA |
| | GCAGCACCCTGACGCTGAGCAAAGCAGACTACGAGAAA |
| | CACAAAGTCTACGCCTGCGAAGTCACCCATCAGGGCCTG |
| | AGCTCGCCCGTCACAAAGAGCTTCAACAGGGGAGAGTG |
| | TTGATGA |

In certain embodiments, the nucleotide sequence of the heavy chain of the anti-CD2 antibody or antigen binding fragment thereof described herein is about 80%, about 85%, about 90%, about 91%, about 92%, about 93%, about 94%, about 95%, about 96%, about 97%, about 98%, about 99%, or 100% identical to SEQ ID NO: 16. In certain embodiments, the nucleotide sequence of the heavy chain of the anti-CD2 antibody or antigen binding fragment thereof described herein is at least about 80%, about 85%, about 90%, about 91%, about 92%, about 93%, about 94%, about 95%, about 96%, about 97%, about 98%, or about 99% identical to SEQ ID NO: 17. In certain embodiments, the nucleotide sequence of the heavy chain of the anti-CD2 antibody or antigen binding fragment thereof described herein is about 80%, about 85%, about 90%, about 91%, about 92%, about 93%, about 94%, about 95%, about 96%, about 97%, about 98%, about 99%, or 100% identical to SEQ ID NO: 16. In certain embodiments, the nucleotide sequence of the heavy chain of the anti-CD2 antibody or antigen binding fragment thereof described herein is at least about 80%, about 85%, about 90%, about 91%, about 92%, about 93%, about 94%, about 95%, about 96%, about 97%, about 98%, or about 99% identical to SEQ ID NO: 17.

In certain embodiments, the VL CDR1, VL CDR2, or VL CDR3 of the anti-CD2 antibody or antigen binding fragment thereof described herein is the CDR that differs from its anti-CD2 Antibody 1 counterpart. In other embodiments, the VH CDR1, VH CDR2, or VH CDR3 of the anti-CD2 antibody or antigen binding fragment thereof described herein is the CDR that differs from its anti-CD2 Antibody 1 counterpart. In certain embodiments, the different CDR of the anti-CD2 antibody or antigen binding fragment thereof described herein is longer than the sequence of its anti-CD2 Antibody 1 counterpart. In other embodiments, the different CDR of the anti-CD2 antibody or antigen binding fragment thereof described herein is shorter than the sequence of its anti-CD2 Antibody 1 counterpart. In certain embodiments, the sequence of the heavy chain constant region (CH) of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 9. In certain embodiments, the sequence of the heavy chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 10. In certain embodiments, the sequence of the heavy chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 11. In certain embodiments, the sequence of the heavy chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 12. In certain embodiments, the sequence of the heavy chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 13. In certain embodiments, the sequence of the heavy chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 30.

In certain embodiments, the sequence of the light chain constant region (CL) of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 14. In certain embodiments, the sequence of the light chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 15. In certain embodiments, the sequence of the light chain constant region of the anti-CD2 antibody or antigen binding fragment thereof described herein is about or at least about 80%, 85%, 90%, 91%, 92%, 93%, 94%, 95%, 96%, 97%, 98%, 99%, or 100% identical to SEQ ID NO: 31.

In certain embodiments, the anti-CD2 antibody or antigen binding fragment thereof described herein can be directed to three regions of human CD2 known in the art as T11₁, T11₂, and T11₃ (Peterson, A., Seed, B., 1987. Nature 329, 842-846; Branco et al., 1999. Transplantation 68, 1588-1596; Arulanandam, A. R., et al., 1993. Proc. Natl. Acad. Sci. U.S.A. 90, 11613-11617; Damschroder et al., 2004. Molecular Immunology 41, 985-1000). Three residues in the adhesion domain of human CD2 that are critical for its binding to siplizumab are N18, K55, and T5 (Damschroder et al., 2004. Molecular Immunology 41, 985-1000). In certain embodiments, the residues N18, K55, and T59 in the extracellular CD2 domain are critical residues in the binding affinity of the anti-CD2 antibody or antigen binding fragment thereof described herein to human CD2. In certain embodiments, the anti-CD2 antibody or antigen binding fragment thereof described herein binds the same epitope as siplizumab. In certain embodiments, the anti-CD2 antibody or antigen binding fragment thereof described herein binds competitively with siplizumab to human CD2.

In certain embodiments, the sequence of the VH region of an anti-CD2 antibody or antigen binding fragment thereof described herein is 80%, 85%, 90%, 91%, 92%, 93%, 94%, 95%, 96%, 97%, 98%, 99%, or 100% identical to one or more of any one of SEQ ID NOs: 22 and 36-40. In certain embodiments, the sequence of the VH region of an anti-CD2 antibody or antigen binding fragment thereof described herein is 80%, 85%, 90%, 91%, 92%, 93%, 94%, 95%, 96%, 97%, 98%, 99%, or 100% identical to SEQ ID NO: 22. In certain embodiments, the sequence of the VH region of an anti-CD2 antibody or antigen binding fragment thereof described herein is 80%, 85%, 90%, 91%, 92%, 93%, 94%, 95%, 96%, 97%, 98%, 99%, or 100% identical to one or more of any one of SEQ ID NOs: 36-40. In certain embodiments, the sequence of the VL region of an anti-CD2 antibody or antigen binding fragment thereof described herein is 80%, 85%, 90%, 91%, 92%, 93%, 94%, 95%, 96%, 97%, 98%, 99%, or 100% identical to one or more of any one of SEQ ID NOs: 23 and 32-35. In certain embodiments, the sequence of the VL region of an anti-CD2 antibody or antigen binding fragment thereof described herein is 80%, 85%, 90%, 91%, 92%, 93%, 94%, 95%, 96%, 97%, 98%, 99%, or 100% identical to SEQ ID NO: 23. In certain embodiments, the sequence of the VL region of an anti-CD2 antibody or antigen binding fragment thereof described herein is 80%, 85%, 90%, 91%, 92%, 93%, 94%, 95%, 96%, 97%, 98%, 99%, or 100% identical to one or more of any one of SEQ ID NOs: 32-35. In certain embodiments, the sequence of the VH CDR1 of an anti-CD2 antibody or antigen binding fragment thereof described herein is SEQ ID NO: 24. In certain embodiments, the sequence of the VH CDR2 of an anti-CD2 antibody or antigen binding fragment thereof described herein is SEQ ID NO: 25. In certain embodiments, the sequence of the VH CDR3 of an anti-CD2 antibody or antigen binding fragment thereof described herein is SEQ ID NO: 26. In certain embodiments, the sequence of the VL CDR1 of an anti-CD2 antibody or antigen binding fragment thereof described herein is SEQ ID NO: 27. In certain embodiments, the sequence of the VL CDR2 of an anti-CD2 antibody or antigen binding fragment thereof described herein is SEQ ID NO: 28. In certain embodiments, the sequence of the VL CDR3 of an anti-CD2 antibody or antigen binding fragment thereof described herein is SEQ ID NO: 29. In certain embodiments, the heavy chain constant region of an anti-CD2 antibody or antigen binding fragment thereof comprises a sequence that is about or at least about 90%, 91%, 92%, 93%, 94%, 95%, 96%, 97%, 98%, 99%, or 100% identical to the amino acid sequence of SEQ ID NO: 30. In certain embodiments, the light chain constant region of an anti-CD2 antibody or antigen binding fragment thereof comprises a sequence that is about or at least about 90%, 91%, 92%, 93%, 94%, 95%, 96%, 97%, 98%, 99%, or 100% identical to the amino acid sequence of SEQ ID NO: 31. In certain embodiments, the heavy chain constant region of an anti-CD2 antibody or antigen binding fragment thereof comprises the amino acid sequence of SEQ ID NO: 30. In certain embodiments, the light chain constant region of an anti-CD2 antibody or antigen binding fragment thereof comprises the amino acid sequence of SEQ ID NO: 31. In certain embodiments, the heavy chain constant region of an anti-CD2 antibody or antigen binding fragment thereof consists of the amino acid sequence of SEQ ID NO: 30. In certain embodiments, the light chain constant region of an anti-CD2 antibody or antigen binding fragment thereof consists of the amino acid sequence of SEQ ID NO: 31. In certain embodiments, an anti-CD2 antibody or antigen binding fragment thereof for use with the methods provided herein has a heavy chain variable region comprising VH CDRs of SEQ ID NOS: 24-26, respectively, and/or a VL of any one of SEQ ID NOs: 23 and 32-35. In certain embodiments an anti-CD2 antibody or antigen binding fragment thereof for use with the methods provided herein has a heavy chain variable region comprising VH CDRs of SEQ ID NOS: 24-26, respectively, and/or a VL of SEQ ID NOs: 23. In certain embodiments, an anti-CD2 antibody or antigen binding fragment thereof for use with the methods provided herein has a heavy chain variable region comprising VH CDRs of SEQ ID NOS: 24-26, respectively, and/or a VL of any one of SEQ ID NOs: 32-35. In certain embodiments, an anti-CD2 antibody or antigen binding fragment thereof for use with the methods provided herein has a heavy chain variable region comprising VL CDRs of SEQ ID NOS: 27-29, respectively, and/or a VH of any one of SEQ ID NO: 22 and 36-40. In certain embodiments, an anti-CD2 antibody or antigen binding fragment thereof for use with the methods provided herein has a heavy chain variable region comprising VL CDRs of SEQ ID NOS: 27-29, respectively, and/or a VH of SEQ ID NO: 22. In certain embodiments, an anti-CD2 antibody or antigen binding fragment thereof for use with the methods provided herein has a heavy chain variable region comprising VL CDRs of SEQ ID NOS: 27-29, respectively, and/or a VH of any one of SEQ ID NO: 36-40.

In certain embodiments, an anti-CD2 antibody or antigen binding fragment thereof for use with the methods provided herein has a heavy chain variable region CDR1 of SEQ ID NO: 24; a heavy chain variable region CDR2 of SEQ ID NO: 25; a heavy chain variable region CDR3 of SEQ ID NO: 26; a light chain variable region CDR1 of SEQ ID NO: 27; a light chain variable region CDR2 of SEQ ID NO: 28; and/or a light chain variable region CDR3 of SEQ ID NO: 29. In certain embodiments, an anti-CD2 antibody or antigen binding fragment thereof for use with the methods provided herein has a heavy chain variable region CDR1 comprising a sequence that is at least about or about 75%, 80%, 85%, 90%, 95%, 96%, 97%, 98%, 99%, or 100% identical to SEQ ID NO: 24; a heavy chain variable region CDR2 comprising a sequence that is at least about or about 75%, 80%, 85%, 90%, 95%, 96%, 97%, 98%, 99%, or 100% identical to SEQ ID NO: 25; a heavy chain variable region CDR3 comprising a sequence that is at least about or about 75%, 80%, 85%, 90%, 95%, 96%, 97%, 98%, 99%, or 100% identical to SEQ ID NO: 26; a light chain variable region CDR1 comprising a sequence that is at least about or about 75%, 80%, 85%, 90%, 95%, 96%, 97%, 98%, 99%, or 100% identical to SEQ ID NO: 27; a light chain variable region CDR2 comprising a sequence that is at least about or about 75%, 80%, 85%, 90%, 95%, 96%, 97%, 98%, 99%, or 100% identical to SEQ ID NO: 28; and/or a light chain variable region CDR3 comprising a sequence that is at least about or about 75%, 80%, 85%, 90%, 95%, 96%, 97%, 98%, 99%, or 100% identical to SEQ ID NO: 29.

In certain embodiments, an anti-CD2 antibody or antigen binding fragment thereof for use with the methods provided herein has a heavy chain variable region CDR1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 24; a heavy chain variable region CDR2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 25; a heavy chain variable region CDR3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 26; a light chain variable region CDR1 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 27; a light chain variable region CDR2 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 28; and/or a light chain variable region CDR3 comprising a sequence that has about or at least about 1 amino acid substitution, about or at least about 2 amino acid substitutions, about or at least about 3 amino acid substitutions, about or at least about 4 amino acid substitutions, or more than at least about 4 amino acid substitutions relative to SEQ ID NO: 29.

In certain embodiments, an anti-CD2 antibody or antigen binding fragment thereof described herein is an IgG1 antibody and has a modification in the Fc region. In certain embodiments, an anti-CD2 antibody or antigen binding fragment thereof described herein is an IgG2 antibody and has a modification in the Fc region. In certain embodiments, an anti-CD2 antibody or antigen binding fragment thereof described herein is an IgG3 antibody and has a modification in the Fc region. In certain embodiments, an anti-CD2 antibody or antigen binding fragment thereof described herein is an IgG4 antibody and has a modification in the Fc region. In certain embodiments, an anti-CD2 antibody or antigen binding fragment thereof described herein comprises at least one mutation (e.g., about or at least one, about or at least two, about or at least three, about or at least four, about or at least five, about or at least six, about or at least seven, about or at least eight, about or at least nine, about or at least ten, or more than about ten mutations) in comparison to anti-CD2 Antibody 1. In certain embodiments, an anti-CD2 antibody or antigen binding fragment thereof described herein comprises about one mutation, about two mutations, about three mutations, about four mutations, about five mutations, about six mutations, about seven mutations, about eight mutations, about nine mutations, about ten mutations, or more than about ten mutations in relation to anti-CD2 Antibody 1 (e.g., mutation in the Fc region of anti-CD2 Antibody 1). In certain embodiments, an anti-CD2 antibody or antigen binding fragment thereof described herein comprises anti-CD2 Antibody 1 CDRs in IgG1 heavy chain and at least one mutation (e.g., about or at least one, about or at least two, about or at least three, about or at least four, about or at least five, about or at least six, about or at least seven, about or at least eight, about or at least nine, about or at least ten, or more than about ten mutations). In certain embodiments, an anti-CD2 antibody or antigen binding fragment thereof described herein comprises anti-CD2 Antibody 1 CDRs in IgG2 heavy chain and at least one mutation (e.g., about or at least one, about or at least two, about or at least three, about or at least four, about or at least five, about or at least six, about or at least seven, about or at least eight, about or at least nine, about or at least ten, or more than about ten mutations). In certain embodiments, an anti-CD2 antibody or antigen binding fragment thereof described herein comprises anti-CD2 Antibody 1 CDRs in IgG3 heavy chain and at least one mutation (e.g., about or at least one, about or at least two, about or at least three, about or at least four, about or at least five, about or at least six, about or at least seven, about or at least eight, about or at least nine, about or at least ten, or more than about ten mutations). In certain embodiments, an anti-CD2 antibody or antigen binding fragment thereof described herein comprises anti-CD2 Antibody 1 CDRs in IgG4 heavy chain and at least one mutation (e.g., about or at least one, about or at least two, about or at least three, about or at least four, about or at least five, about or at least six, about or at least seven, about or at least eight, about or at least nine, about or at least ten, or more than about ten mutations). The mutation can comprise of at least one alteration in the amino acid sequence of an anti-CD2 antibody or antigen binding fragment thereof as compared to the wild-type counterpart wherein the alteration results in the reduction or elimination of the binding of the Fc region to its cognate receptor. In some embodiments, a mutation is one or more mutation and includes a mutation in amino acid position L234, L235, P329, V234, G237, P238, H268, V309, A330, P331, and/or S228 (e.g., based on Edelman (EU) numbering). In some embodiments, a mutation is one or more mutation and includes a mutation in amino acid position L234 and/or L235. In some embodiments, a mutation is one or more mutation and includes a mutation in amino acid position L234, L235, and/or P329. In some embodiments, a mutation is one or more mutation and includes a mutation in amino acid position V234, G237, P238, H268, V309, A330, and/or P331. In some embodiments, a mutation is one or more mutation and includes a mutation in amino acid position S228. In some embodiments, a mutation is one or more mutation and includes a mutation in amino acid position S228, P329, and/or L235. In some embodiments, a mutation is one or more mutation and includes L234A (or another conservative amino acid mutation). In some embodiments, a mutation is one or more mutation and includes L235A (or another conservative amino acid mutation). In some embodiments, a mutation is one or more mutation and includes P329G (or another conservative amino acid mutation). In some embodiments, a mutation is one or more mutation and includes V234A (or another conservative amino acid mutation). In some embodiments, a mutation is one or more mutation and includes G237A (or another conservative amino acid mutation). In some embodiments, a mutation is one or more mutation and includes P238S (or another conservative amino acid mutation). In some embodiments, a mutation is one or more mutation and includes H268A (or another conservative amino acid mutation). In some embodiments, a mutation is one or more mutation and includes V309L (or another conservative amino acid mutation). In some embodiments, a mutation is one or more mutation and includes A330S (or another conservative amino acid mutation). In some embodiments, a mutation is one or more mutation and includes P331S (or another conservative amino acid mutation). In some embodiments, a mutation is one or more mutation and includes S228P (or another conservative amino acid mutation). In some embodiments, a mutation is one or more mutation and includes L235E (or another conservative amino acid mutation). In some embodiments, the mutation includes L234A and L235A (or other conservative amino acid mutations). In some embodiments, the mutation includes P329G, L234A and L235A (or other conservative amino acid mutations). In some embodiments, the mutation includes V234A, G237A, P238S, H268A, V309L, A330S, and P331S (or other conservative amino acid mutations) (e.g., for IgG2). In some embodiments, a mutation is one or more mutation and includes S228P (or another conservative amino acid mutation) (e.g., for IgG4). In some embodiments, a mutation is one or more mutation and includes P329G, S228P, and L235E (or another conservative amino acid mutations) (e.g., for IgG4). In some embodiments, the amino acid position is a position based on any antibody numbering scheme (e.g., Edelman (EU) numbering). In some embodiments, the amino acid position is a position based on Edelman (EU) numbering. In some embodiments, the amino acid position is a position based on Kabat numbering scheme. In some embodiments, the amino acid position is a position based on Clothia numbering scheme. In some embodiments, the amino acid position is a position based on IMGT numbering scheme.

The modification can comprise of at least one alteration in the amino acid sequence of the anti-CD2 antibody or antigen binding fragment thereof Fc region as compared to the wild-type Fc region wherein the alteration results in the reduction or elimination of the binding of the Fc region to its cognate receptor. The Fc receptor is located on immune effectors cells including B cells, NK cells, macrophages, and neutrophils. Without being bound by any particular theory, in wild type IgG, the Fc interaction with the Fc receptor (FcR) leads to downstream effector cell functions including stimulating the phagocytic or cytotoxic activities of the immune cell. Reduction or elimination of Fc/FcR interaction results in elimination of effector functions.

In certain embodiments, the anti-CD2 antibody or antigen binding fragment thereof exhibits reduced binding to the FcγRIIIA receptor as compared to anti-CD2 Antibody 1. In certain embodiments, an anti-CD2 antibody or antigen binding fragment thereof exhibits reduced binding to the FcγRIIA receptor as compared to anti-CD2 Antibody 1. In certain embodiments, an anti-CD2 antibody or antigen binding fragment thereof exhibits reduced binding to the FcγRI receptor as compared to anti-CD2 Antibody 1. In certain embodiments, an anti-CD2 antibody or antigen binding fragment thereof) exhibits increased binding to the FcγRIIIA receptor as compared to anti-CD2 Antibody 1. In certain embodiments, an anti-CD2 antibody or antigen binding fragment thereof exhibits increased binding to the FcγRIIA receptor as compared to anti-CD2 Antibody 1. In certain embodiments, an anti-CD2 antibody or antigen binding fragment thereof exhibits increased binding to the FcγRI receptor as compared to anti-CD2 Antibody 1. In certain embodiments, an anti-CD2 antibody or antigen binding fragment thereof as described herein binds at about, at least about, or at most about 0%, 5%, 10%, 15%, 20%, 25%, 30%, 35%, 40%, 45%, 50%, 55%, 60%, 65%, 70%, 75%, 80%, 85%, 90%, or 95% of the binding ability exhibited by anti-CD2 Antibody 1. In certain embodiments, an anti-CD2 antibody or antigen binding fragment thereof as described herein has about or at least about 5%, 10%, 15%, 20%, 25%, 30%, 35%, 40%, 45%, 50%, 55%, 60%, 65%, 70%, 75%, 80%, 85%, 90%, or 95% higher binding ability (e.g., to FcγRIIA, FcγRIIIA, and/or FcγRI) as compared to the binding ability exhibited by anti-CD2 Antibody 1 (e.g., to FcγRIIA, FcγRIIIA, and/or FcγRI). In certain embodiments, an anti-CD2 antibody or antigen binding fragment thereof as described herein has about or at least about 5%, 10%, 15%, 20%, 25%, 30%, 35%, 40%, 45%, 50%, 55%, 60%, 65%, 70%, 75%, 80%, 85%, 90%, or 95% lower binding ability (e.g., to FcγRIIA, FcγRIIIA, and/or FcγRI) as compared to the binding ability exhibited by anti-CD2 Antibody 1 (e.g., to FcγRIIA, FcγRIIIA, and/or FcγRI). Assays to detect binding events can include, but are not limited to, Enzyme-Linked Immunosorbent Assays (ELISAs) and/or surface plasmon resonance (SPR) methods such as the Biacore system.

In certain embodiments, the Fc region can be modified by any appropriate method known in the art. In certain embodiments, a modification can result in Fc silencing. In certain embodiments, the modification can include the mutation of the amino acid sequence of the IgG Fc. In certain embodiments, the modification can include the mutation of the glycosylation site (N297) or of the consensus sequence comprising N297. In certain embodiments, modifications can include mutations that inhibit FcγR and C1q binding. In certain embodiments, these mutations can include any or all of the mutations K322A, L234A and L235A. In certain embodiments, an anti-CD2 antibody or antigen binding fragment thereof described herein can be a Fab, wherein no Fc is present. In certain embodiments, an anti-CD2 antibody or antigen binding fragment thereof described herein is an IgG4 antibody. Without being bound by any particular theory, the IgG4 subclass is desirable for therapeutic purposes due to the lack of effector functions including, but not limited to ADCC (Davies and Sutton, Immunol Rev. 2015 November; 268 (1): 139-15). In certain embodiments, an anti-CD2 antibody or antigen binding fragment thereof as described herein is an IgG2 antibody. Without being bound by any particular theory, beneficial characteristics of the IgG2 subclass include that IgG2 does not cross placenta (Einarsdottir et al., PLoS One. 2014 Sep. 24; 9 (9):e108319) and that IgG2 has very low/no Fc receptor binding capacity (Vidarsson et al., Front Immunol. 2014; 5:520).

In certain embodiments, the anti-CD2 antibody or antigen binding fragment thereof described herein can have an antigen binding variable region and a Fc region. In certain embodiments, the Fc region of the anti-CD2 antibody or antigen binding fragment thereof described herein contains a glycosylation consensus sequence in each of the heavy chains of the antibody. In certain embodiments, the glycosylation consensus sequence is Asn-X-Ser. In certain embodiments, the glycosylation consensus sequence is Asn-X-Thr. In certain embodiments, the anti-CD2 antibody or antigen binding fragment thereof described herein is glycosylated at Asn297. In certain embodiments, the anti-CD2 antibody or antigen binding fragment thereof described herein has been deglycosylated. In certain embodiments, the glycan attached to the asparagine residue can be an N-linked glycan. In certain embodiments, the glycan attached to the asparagine residue can be an O-linked glycan. In a specific embodiment, the anti-CD2 antibody or antigen binding fragment thereof described herein can contain a single N-linked glycosylation site on Asn297 of the heavy chain.

In certain embodiments, the anti-CD2 antibody or antigen binding fragment thereof described herein can be glycosylated at the Fc glycosylation consensus sequence. In certain embodiments, the anti-CD2 antibody or antigen binding fragment thereof described herein is deglycosylated. In a certain embodiment, the deglycosylation of the anti-CD2 antibody or antigen binding fragment thereof described herein is achieved by modifying the Fc region, specifically by introducing a point mutation at position N297. In a certain embodiment, the mutation introduced at position 297 (N297) can be, but is not limited to, N297G, N297Q, or N297A. Without being bound by any particular theory, the N297 point mutation can result in the lack of glycosylation and silencing of Fc signaling. In certain embodiments, the deglycosylation of the anti-CD2 antibody or antigen binding fragment thereof described herein is achieved by chemical or enzymatic degradation of the anti-CD2 antibody or antigen binding fragment thereof glycan structures. In a specific embodiment, the chemical or enzymatic methods of glycan degradation preserves the Fc amino acid sequence.

In certain embodiments, the constant region of the anti-CD2 antibody or antigen binding fragment thereof described herein can be switched with the constant region of an antibody of a different subclass. Without being bound by any particular theory, while the variable region does not change, a subclass-switched antibody retains its specific affinity while interacting with different effector molecules (Valenzuela and Schaub, Transplantation. 2018 January; 102 (1S Suppl 1): S7-S13). In a certain embodiment, the constant region of the anti-CD2 antibody or antigen binding fragment thereof described herein can be switched with a constant region of a different antibody. In a certain embodiment, this switch can result in the anti-CD2 antibody or antigen binding fragment thereof becoming a different subclass of antibody than it was originally. In certain embodiments, the constant region of the anti-CD2 antibody or antigen binding fragment thereof can be switched with a different antibody while preserving the specific binding of the variable regions. In certain embodiments, the constant region of the anti-CD2 antibody or antigen binding fragment thereof can be switched with a different antibody and the specific variable region is preserved, wherein the VH region of the anti-CD2 antibody or antigen binding fragment thereof is about or at least about 80%, 85%, 90%, 91%, 92%, 93%, 94%, 95%, 96%, 97%, 98%, 99%, or 100% identical to SEQ ID NO: 1, and the sequence of the VL region of the anti-CD2 antibody or antigen binding fragment thereof is about or at least about 80%, 85%, 90%, 91%, 92%, 93%, 94%, 95%, 96%, 97%, 98%, 99%, or 100% identical to SEQ ID NO: 2.

In certain embodiments, the anti-CD2 antibody or antigen binding fragment thereof binds specifically to the same epitope in human CD2 as anti-CD2 Antibody 1. In certain embodiments, the anti-CD2 antibody or antigen binding fragment thereof binds specifically to the same epitope in human CD2 as siplizumab. In certain embodiments, the anti-CD2 antibody or antigen binding fragment thereof can be an animal-specific antibody, a human-specific antibody, a chimeric antibody, a humanized antibody, a full length antibody, an antibody fragment, a single chain variable fragment (scFv), a naturally occurring antibody, a synthetic antibody, an engineered antibody, enlarged anti-CD2 variants wherein additional components are added to the Fc region (e.g., a component can include an scFv, a CH2 domain, and/or a CH3 domain), or a combination thereof. In certain embodiments, the antibody Fc region has a point mutation (e.g., in N297) resulting in Fc silencing. In certain embodiments, the antibody is an IgG1. In certain embodiments, the antibody is an IgG2. In certain embodiments, the antibody is an IgG4. In certain embodiments, the antibody has a different native constant region than anti-CD2 Antibody 1. In certain embodiments, the antibody has a different native constant region than anti-CD2 Antibody 1 and a point mutation in the Fc region resulting in Fc silencing. In some embodiments, the anti-CD2 antibody or antigen binding fragment thereof is a humanized anti-CD2 monoclonal antibody. In a specific embodiment, the anti-CD2 antibody or antigen binding fragment thereof is siplizumab. In certain embodiments, the anti-CD2 antibody or antigen binding fragment thereof as described herein can have a modified Fc region wherein the modification can include, but is not limited to, a point mutation resulting in Fc silencing, a switched native constant region, or an Fc silenced switched new native constant region. These combinations of different antibody subclasses and modifications produce different versions of the anti-CD2 antibody or antigen binding fragment thereof which are outlined in Table 3. In certain embodiments, the anti-CD2 antibody or antigen binding fragment thereof as described herein can be an IgG1, an IgG2, or an IgG4 subclass of antibody.

TABLE 3
Examples of versions of anti-CD2 antibody
or antigen binding fragment thereof

| IgG | Modification of CD2-binding molecule (or anti-CD2 |
|---|---|
| subclass | antibody or antigen binding fragment thereof) |
| IgG1 | A point mutation resulting in silenced Fc |
| IgG1 | same variable region but combined with a different native |
| | constant region |
| IgG1 | same variable region but combined with a Fc-silenced new |
| | native constant region |
| IgG2 | same variable region but combined with a different native |
| | constant region |
| IgG2 | same variable region but combined with a Fc-silenced new |
| | native constant region |
| IgG4 | same variable region but combined with a different native |
| | constant region |
| IgG4 | same variable region but combined with a Fc-silenced new |
| | native constant region |

Comparisons of anti-CD2 antibody or antigen binding fragment thereof are outlined in Table 4.

TABLE 4
Comparisons of anti-CD2 antibody or
antigen binding fragment thereof

| | VH | VL | | Fc | IgG |
|---|---|---|---|---|---|
| Molecule | region: | region: | Deglycosylated | modification | subclass |
| CD2-binding | SEQ ID | SEQ ID | Yes | None | IgG1 |
| molecule 1 | NO: 1 | NO: 2 | | | |
| CD2-binding | SEQ ID | SEQ ID | No | LALA-PG | IgG1 |
| molecule 2 | NO: 1 | NO: 2 | | mutations | |
| CD2-binding | SEQ ID | SEQ ID | No | V234A, | IgG2 |
| molecule 3 | NO: 1 | NO: 2 | | G237A, | |
| | | | | P238S, | |
| | | | | H268A, | |
| | | | | V309L, | |
| | | | | A330S, | |
| | | | | P331S | |
| CD2-binding | SEQ ID | SEQ ID | No | S228P, | IgG4 |
| molecule 4 | NO: 1 | NO: 2 | | L235E, | |
| | | | | P329G | |

In certain embodiments, the anti-CD2 antibody or antigen binding fragment thereof described herein, including examples presented above, have little to no ADCC activity compared to anti-CD2 Antibody 1, yet retain the immunomodulatory effects exhibited by anti-CD2 Antibody 1. In certain embodiments, the anti-CD2 antibody or antigen binding fragment thereof provided herein can have a molecular weight that is higher than the molecular weight of an unmodified IgG1 antibody. In certain embodiments, this increase of molecular weight can be accomplished by the addition of duplicate regions of the molecule. In certain embodiments, the regions can be attached to the anti-CD2 antibody or antigen binding fragment thereof through such means known in the art such as, but not limited to, chemical conjugation, recombinant fusion, and covalent attachment.

In certain embodiments, the additional regions to be attached to the anti-CD2 antibody or antigen binding fragment thereof, can include, but are not limited to, duplicate regions of the molecule such as an additional variable heavy chain (VH), an additional variable light chain (VL), a scFv comprising the fusion of an additional VH and an additional VL, an additional CH2 domain, or an additional CH3 domain. In certain embodiments, the additional regions can be attached to the Fc region of the anti-CD2 antibody or antigen binding fragment thereof.

In certain embodiments, an scFv can be attached to the Fc region of the anti-CD2 antibody or antigen binding fragment thereof. In certain embodiments, the scFv can comprise a fusion of the VH and the VL of anti-CD2 Antibody 1. In certain embodiments, the scFv can comprise a fusion of a VH, wherein the VH can comprise an amino acid sequence of SEQ ID NO: 1, and a VL, wherein the VL can comprise an amino acid sequence of SEQ ID NO: 2. In a specific embodiment, the scFv binds to CD2. In certain embodiments, the CDRs of the scFv are the same as the CDRs of anti-CD2 Antibody 1. In certain embodiments, the scFv attached to the Fc region of the anti-CD2 antibody or antigen binding fragment thereof described herein binds to an epitope on CD2. In certain embodiments, the scFv attached to the Fc region of the anti-CD2 antibody or antigen binding fragment thereof described herein binds to the same epitope of CD2 as anti-CD2 Antibody 1. In certain embodiments, the scFv can be generated from an IgG2 antibody or an IgG4 antibody.

In certain embodiments, an additional CH2 domain can be attached to the Fc. In certain embodiments, the CH2 domain can comprise an amino acid sequence that is about or at least about 80%, 85%, 90%, 95%, 98%, or 99% identical to the amino acid sequence of SEQ ID NO: 18 or 19. In certain embodiments, the additional CH2 domain can be generated from an IgG2 antibody or an IgG4 antibody. In certain embodiments, an additional CH3 domain can be attached to the Fc. In certain embodiments, the CH3 domain can comprise an amino acid sequence that is about or at least about 80%, 85%, 90%, 95%, 98%, or 99% identical to the amino acid sequence of SEQ ID NO: 20 or 21. In certain embodiments, the additional CH3 domain can be generated from an IgG2 antibody or an IgG4 antibody. In a certain embodiment, the additional domains can be attached to the C-terminal end of the Fc. In certain embodiments, the domains can be attached to the anti-CD2 antibody or antigen binding fragment thereof through such means known in the art such as, but not limited to, chemical conjugation, recombinant fusion, and covalent attachment. In certain embodiments, the domains can be attached to the anti-CD2 antibody or antigen binding fragment thereof through such means known in the art such as, but not limited to, chemical conjugation, recombinant fusion, and covalent attachment. Examples of CD2 antibody or antigen binding fragment thereof that can be used in disclosure and production thereof are described, for example, in WO 2021/259927, which is incorporated herein by reference in its entirety.

In certain embodiments, the anti-CD2 antibody or antigen binding fragment thereof described herein can comprise a VH of SEQ ID NO: 1, a VL of SEQ ID NO: 2, a CH of SEQ ID NO: 9, and a CL of SEQ ID NO: 14; a VH of SEQ ID NO: 1, a VL of SEQ ID NO: 2, a CH of SEQ ID NO: 10, and a CL of SEQ ID NO: 14; a VH of SEQ ID NO: 1, a VL of SEQ ID NO: 2, a CH of SEQ ID NO: 11, and a CL of SEQ ID NO: 14; a VH of SEQ ID NO: 1, a VL of SEQ ID NO: 2, a CH of SEQ ID NO: 12, and a CL of SEQ ID NO: 14; or a VH of SEQ ID NO: 1, a VL of SEQ ID NO: 2, a CH of SEQ ID NO: 13, and a CL of SEQ ID NO: 14. In certain embodiments, the anti-CD2 antibody or antigen binding fragment thereof described herein can comprise a VH of SEQ ID NO: 1, a VL of SEQ ID NO: 2, a CH of SEQ ID NO: 9, and a CL of SEQ ID NO: 15; a VH of SEQ ID NO: 1, a VL of SEQ ID NO: 2, a CH of SEQ ID NO: 10, and a CL of SEQ ID NO: 15; a VH of SEQ ID NO: 1, a VL of SEQ ID NO: 2, a CH of SEQ ID NO: 11, and a CL of SEQ ID NO: 15; a VH of SEQ ID NO: 1, a VL of SEQ ID NO: 2, a CH of SEQ ID NO: 12, and a CL of SEQ ID NO: 15; or a VH of SEQ ID NO: 1, a VL of SEQ ID NO: 2, a CH of SEQ ID NO: 13, and a CL of SEQ ID NO: 15.

In certain embodiments, the anti-CD2 antibody or antigen binding fragment thereof described herein can comprise a heavy chain of SEQ ID NO: 16; a VL of SEQ ID NO: 2, and a CL of SEQ ID NO: 14; a heavy chain of SEQ ID NO: 16; a VL of SEQ ID NO: 2, and a CL of SEQ ID NO: 15; a VH of SEQ ID NO: 1; a CH of SEQ ID NO: 9; and a light chain of SEQ ID NO: 17; a VH of SEQ ID NO: 1; a CH of SEQ ID NO: 10; and a light chain of SEQ ID NO: 17; a VH of SEQ ID NO: 1; a CH of SEQ ID NO: 11; and a light chain of SEQ ID NO: 17; a VH of SEQ ID NO: 1; a CH of SEQ ID NO: 12; and a light chain of SEQ ID NO: 17; a VH of SEQ ID NO: 1; a CH of SEQ ID NO: 13; and a light chain of SEQ ID NO: 17; or a heavy chain of SEQ ID NO: 16 and a light chain of SEQ ID NO: 17.

In certain embodiments, the anti-CD2 antibody or antigen binding fragment thereof may comprise enlarged variants. In certain embodiments, the Fc of the anti-CD2 antibody or antigen binding fragment thereof described herein comprises an additional region such that the molecular weight of the anti-CD2 antibody or antigen binding fragment thereof is increased by 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa, or 60 kDa. In certain embodiments, the Fc of the anti-CD2 antibody or antigen binding fragment thereof described herein comprises an additional region such that the molecular weight of the anti-CD2 antibody or antigen binding fragment thereof is increased by a range of 10 kDa to 15 kDa, 11 kDa to 16 kDa, 12 kDa to 17 kDa, 13 kDa to 18 kDa, 14 kDa to 19 kDa, 15 kDa to 20 kDa, 16 kDa to 21 kDa, 17 kDa to 22 kDa, 18 kDa to 23 kDa, 19 kDa to 24 kDa, 20 kDa to 25 kDa, 21 kDa to 26 kDa, 22 kDa to 27 kDa, 23 kDa to 28 kDa, 24 kDa to 29 kDa, 25 kDa to 30 kDa, 26 kDa to 31 kDa, 27 kDa to 32 kDa, 28 kDa to 33 kDa, 29 kDa to 34 kDa, 30 kDa to 35 kDa, 31 kDa to 36 kDa, 32 kDa to 37 kDa, 33 kDa to 38 kDa, 34 kDa to 39 kDa, 35 kDa to 40 kDa, 36 kDa to 41 kDa, 37 kDa to 42 kDa, 38 kDa to 43 kDa, 39 kDa to 44 kDa, 40 kDa to 45 kDa, 41 kDa to 46 kDa, 42 kDa to 47 kDa, 43 kDa to 48 kDa, 44 kDa to 49 kDa, 45 kDa to 50 kDa, 46 kDa to 51 kDa, 47 kDa to 52 kDa, 48 kDa to 53 kDa, 49 kDa to 54 kDa, 50 kDa to 55 kDa, 51 kDa to 56 kDa, 52 kDa to 57 kDa, 53 kDa to 58 kDa, 54 kDa to 59 kDa, or 55 kDa to 60 kDa.

In certain embodiments, duplicate regions of the anti-CD2 antibody or antigen binding fragment thereof can be attached to the molecule to produce enlarged variants. In certain embodiments, these regions can be attached to the Fc region of the anti-CD2 antibody or antigen binding fragment thereof through such means known in the art such as, but not limited to, chemical conjugation, recombinant fusion, and covalent attachment.

In certain embodiments, the enlarged variants of the anti-CD2 antibody or antigen binding fragment thereof can be derived from an IgG1, IgG2, or IgG4. In certain embodiments, an additional scFv can be attached to the Fc of the anti-CD2 antibody or antigen binding fragment thereof to produce an enlarged variant. In certain embodiments, the scFv can comprise a VH and VL wherein the VH can comprise an amino acid sequence that is about or at least about 80%, 85%, 90%, 95%, 98%, or 99% identical to the amino acid sequence of SEQ ID NO: 1, and wherein the VL can comprise an amino acid sequence that is about or at least about 80%, 85%, 90%, 95%, 98%, or 99% identical to the amino acid sequence of SEQ ID NO: 2.

In certain embodiments, an additional CH2 domain can be attached to the Fc of the anti-CD2 antibody or antigen binding fragment thereof to produce an enlarged variant. In certain embodiments, the additional CH2 domain can comprise an amino acid sequence that is about or at least about 80%, 85%, 90%, 95%, 98%, or 99% identical to the amino acid sequence of SEQ ID NO: 18 or 19. In certain embodiments, an additional CH3 domain can be attached to the Fc of the anti-CD2 antibody or antigen binding fragment thereof to produce an enlarged variant. In certain embodiments, the additional CH3 domain can comprise an amino acid sequence that is about or at least about 80%, 85%, 90%, 95%, 98%, or 99% identical to the amino acid sequence of SEQ ID NO: 20 or 21. In some embodiments, the additional fragment or domains can be attached to the C-terminal end of the Fc. In certain embodiments, the enlarged anti-CD2 variant can be an IgG2 antibody. In certain embodiments, the enlarged anti-CD2 variant can be an IgG4 antibody. The details of enlarged anti-CD2 variants are shown in Table 5.

In certain embodiments, the additional region attached to the anti-CD2 antibody or antigen binding fragment thereof can comprise an unrelated or artificial amino acid sequence. In certain embodiments, the additional region attached to the anti-CD2 antibody or antigen binding fragment thereof can comprise a fragment, such as a structural domain, from another human protein. In certain embodiments, the additional region attached to the anti-CD2 antibody or antigen binding fragment thereof can comprise a fragment of a domain from another human protein. In certain embodiments, the addition of such an additional region does not interfere with the binding activity of the parent anti-CD2 antibody or antigen binding fragment thereof and/or does not increase the immunogenicity of the resulting anti-CD2 binder relative to the parent anti-CD2 antibody or antigen binding fragment thereof in humans.

TABLE 5
Enlarged anti-CD2 antibody variants

| | | Additional antibody | Theoretical protein |
|---|---|---|---|
| Description | IgG subclass | component | weight |
| Molecule 5A | IgG2 | scFv | 199.4 kDa |
| Molecule 5B | IgG4 | scFv | 199.6 kDa |
| Molecule 6A | IgG2 | CH2 domain | 170.1 kDa |
| Molecule 6B | IgG4 | CH2 domain | 170.3 kDa |
| Molecule 7A | IgG2 | CH3 domain | 171.2 kDa |
| Molecule 7B | IgG4 | CH3 domain | 171.4 kDa |

In certain embodiments, the anti-CD2 antibody or antigen binding fragment thereof is a humanized monoclonal antibody. In certain embodiments, the anti-CD2 antibody or antigen binding fragment thereof is anti-CD2 Antibody 1. In a specific embodiment, the anti-CD2 antibody or antigen binding fragment thereof can be siplizumab (previously known as MEDI-507). In certain embodiments, the administration of the anti-CD2 antibody or antigen binding fragment thereof can be modified as described herein to achieve and/or maintain mixed chimerism.

In certain embodiments, the anti-CD2 antibody or antigen binding fragment thereof disclosed herein can be modified to increase its permeability across the blood brain barrier (BBB). In some embodiments, the anti-CD2 antibody or antigen binding fragment thereof disclosed herein can be modified to include at least one molecule that binds with receptors on the BBB. In some embodiments, the anti-CD2 antibody or antigen binding fragment thereof disclosed herein can be modified to be a bispecific antibody comprising at least one antigen for an endogenous receptor expressed on the luminal side of the BBB. Non-limiting examples of endogenous receptors expressed the luminal side of the BBB include receptors for insulin, transferrin, insulin-like growth factors 1 and 2 (IGF1 and IGF2), leptin, and/or lipoproteins.

In certain embodiments, the anti-CD2 antibody or antigen binding fragment thereof can be administered to a subject in a convenient manner known in the art including subcutaneously, intravenously, intravascularly, topically, intra-arterially, intra-cranially, intramuscularly, orally, intra-orbitally, by inhalation, transdermally, intra-peritonially, or any combination thereof. In a specific embodiment, the anti-CD2 antibody or antigen binding fragment thereof is administered intravenously. In a specific embodiment, the anti-CD2 antibody or antigen binding fragment thereof is administered subcutaneously.

In certain embodiments, a method described herein comprises administration of the anti-CD2 antibody or antigen binding fragment thereof and one or more agents to treat or prevent a side effect of the anti-CD2 antibody or antigen binding fragment thereof and/or of ALS (e.g., acetaminophen, diphenhydramine, corticosteroids, antihistamines, epinephrine, bronchodilators, oxygen, nonsteroidal anti-inflammatory drug, oral analgesics, antihistamine, and/or immunosuppressant).

### 7.1.1.2 Production of Anti-CD2 Antibody or an Antigen Binding Fragment Thereof

Provided herein are methods for producing the anti-CD2 antibody or an antigen binding fragment thereof as described herein, wherein the anti-CD2 antibody or antigen binding fragment thereof is effective in treating or preventing ALS when administered to a subject in need thereof. Methods for producing the anti-CD2 antibody or antigen binding fragment thereof described herein include recombinant expression techniques, selection methods, transformation into host cells, and purification of the anti-CD2 antibody or antigen binding fragment thereof (such as the methods described in Section 7.1.1.2 (i)). Also provided herein are methods for producing fragments of the anti-CD2 antibody (such as the methods described in Section 7.1.1.2 (ii)), deglycosylated anti-CD2 antibody or antigen binding fragment thereof (such as the anti-CD2 antibody or antigen binding fragment thereof described in Section 7.1.1.2 (iii)), and cells comprising vectors for recombinantly expressing the anti-CD2 antibody or antigen binding fragment thereof described herein (such as the cells described in Section 7.1.1.2 (iv)).

### (i) Recombinant Expression Systems

Described herein are methods for producing the anti-CD2 antibody or an antigen binding fragment thereof of the present disclosure. The anti-CD2 antibody or an antigen binding fragment thereof described herein can be produced by any appropriate expression method known in the art. These methods of producing antibodies can include, but are not limited to, the use of hybridoma cells, bacteria, yeast cells, insect cells, transgenic animals, transgenic plants, phage display, ribosome display, and recombinant expression in mammalian cell lines. In certain embodiments, the method to produce the recombinant anti-CD2 antibody can include isolation, amplifying, and cloning of the VH and VL genes of the antibody; the joining together of the heavy and light fragments; and then cloning the chains into special phage vectors. Upon integration, antibodies are displayed on the cell surface and the antibody with desired binding specificity can be selected for by using any appropriate selection method known in the art. These methods can include, but are not limited to, panning, use of paramagnetic beads, fluorescence-activated cell sorting (FACS), Enzyme-Linked Immunosorbent Assays (ELISAs), and/or surface plasmon resonance (SPR) methods such as the Biacore system.

After selection, the genes for the antibody can be transferred into an expression vector. In certain embodiments, a strong promoter is also inserted into the expression vector. The promoter can be a viral promoter, such as CMV or SV40; or a non-viral promoter such as elongation factor (EF)-1 promotor, UBC, PGK, or CAG promoter. The expression vector is transformed into a host cell line and integrated into the host cell genome. In certain embodiments, the host cell line can include bacteria, yeast, or mammalian cell lines. In a specific embodiment, the mammalian cell lines that can be used as a host cell line for recombinant antibody production includes, but are not limited to, Chinese hamster ovary (CHO) cell line, NSO cell line, Sp2/0 cell line, PER. C6 cell line, and human embryonic kidney (HEK) cell line.

In certain embodiments, the anti-CD2 antibody or antigen binding fragment thereof described herein can be recovered from the host cell cultures and purified by any appropriate method known in the art. Standard antibody purification methods include, but are not limited to, protein-A affinity chromatography, Q-sepharose anion exchange chromatography, sulfopropyl-sepharose cation exchange chromatography, high performance liquid chromatography (HPLC), protein G purification, ammonium sulfate or ethanol precipitation, acid extraction, phosphocellulose chromatography, hydrophobic interaction chromatography, affinity chromatography, hydroxylapatite chromatography and lectin chromatography.

### (ii) Production of Antigen Binding Fragment of Anti-CD2 Antibody

In addition to the procedural steps described above, modifications or additional steps can be taken to produce antigen binding fragment of anti-CD2 antibody. These CD2-binding molecule fragments can include, but are not limited to, Fabs, F(ab′) 2, scFv, and single-domain antibodies (sdAbs). In certain embodiments, the procedure to produce a CD2-binding scFv includes isolation, amplifying, and cloning of the VH and VL genes of the antibody in addition to a linker. A flexible peptide linker is needed to join the VH and VL fragments. In certain embodiments, the procedure to produce CD2-binding sdAb includes isolation, amplifying, and cloning of camelid or shark VH region, that results in a sole VH region lacking a paired VL region, attached to a constant region.

In certain embodiments, the anti-CD2 antibody described herein can be treated with papain to produce Fab binding fragments. In certain embodiments, the anti-CD2 antibody described herein can be treated with pepsin to produce F(ab′)₂. In a specific embodiment, the sequence of the VH region of the Fab, F(ab′)₂, scFv, or sdAb described herein is 80%, 85%, 90%, 91%, 92%, 93%, 94%, 95%, 96%, 97%, 98%, 99%, or 100% identical to SEQ ID NO: 1. In a specific embodiment, the sequence of the VL region of the Fab, F(ab′) 2, scFv, or sdAb described herein is 80%, 85%, 90%, 91%, 92%, 93%, 94%, 95%, 96%, 97%, 98%, 99%, or 100% identical to SEQ ID NO: 2.

(iii) Deglycosylation of Anti-CD2 Antibody

Described herein are methods for the production of a deglycosylated anti-CD2 antibody or antigen binding fragment thereof of the present disclosure. In some embodiments, the glycans of the anti-CD2 antibody or antigen binding fragment thereof can be modified using chemical methods, such as treatment with trifluoromethanesulfonic (TFMS) acid. In some embodiments, the glycans of the anti-CD2 antibody or antigen binding fragment thereof can be modified using enzymatic methods. In a specific embodiment, the chemical or enzymatic methods of glycan degradation preserves the Fc amino acid sequence of the anti-CD2 antibody or antigen binding fragment thereof described herein.

Enzymes that can be used to modify the glycans of the anti-CD2 antibody or antigen binding fragment thereof described herein can include but are not limited to Peptide-N-Glycosidase F (PNGase F), Peptide-N-Glycosidase A (PNGase A), endoglycosidase H, endoglycosidase F, endo-β-N-acetylglucosaminidase (EndoS), endo-α-N-acetylgalactosaminidase, sialidase A, β1-4 galactosidase S, β-N-acetylhexosaminidase, and EndoS2. In a certain embodiment, the anti-CD2 antibody or antigen binding fragment thereof described herein is treated with an endoglycosidase. In a specific embodiment, the endoglycosidase used to degrade the glycan structures of the anti-CD2 antibody or antigen binding fragment thereof described herein is EndoS (Sjogren et al., Glycobiology, 2015, vol. 25, no. 10, 1053-1063). In a specific embodiment, the endoglycosidase used to degrade the glycan structures of the anti-CD2 antibody or antigen binding fragment thereof described herein is EndoS2 (Sjogren et al., Biochem. J. (2013) 455, 107-118).

EndoS and EndoS2, secreted from Streptococcus pyogenes, specifically remove N-linked glycans from the chitobiose core of the heavy chain of human IgG. Incubation with endoglycosidase EndoS2 from Streptococcus pyogenes results in cleavage between the two GlcNAc residues in the chitobiose core of N-glycans, while leaving the core GlcNAc intact. In certain embodiments, the anti-CD2 antibody or antigen binding fragment thereof described herein is treated with EndoS or EndoS2. Both EndoS and EndoS2 remove complex glycans while EndoS2 removes high-mannose glycans.

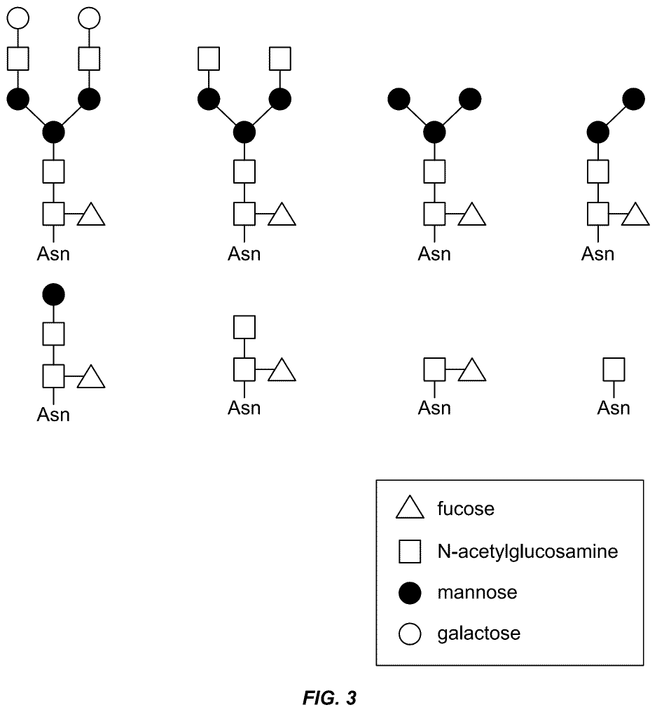

In certain embodiments, the anti-CD2 antibody or antigen binding fragment thereof described herein is an IgG antibody. In certain embodiments, the anti-CD2 antibody or antigen binding fragment thereof described herein has at least one consensus N-linked glycosylation site of the constant region of the heavy chain. In certain embodiments, the anti-CD2 antibody or antigen binding fragment thereof described herein carries at least one of the following oligosaccharides illustrated in FIG. 3. In a specific embodiment, the anti-CD2 antibody or antigen binding fragment thereof described herein is treated with EndoS. In a specific embodiment, the anti-CD2 antibody or antigen binding fragment thereof described herein is treated with EndoS2. In a specific embodiment, the anti-CD2 antibody or antigen binding fragment thereof described herein is deglycosylated. In a specific embodiment, the anti-CD2 antibody or antigen binding fragment thereof described herein is an IgG antibody, wherein the ability of the antibody to mediate antibody-dependent cellular cytotoxicity is reduced or eliminated while the immune regulatory activity of the antibody is maintained relative to anti-CD2 Antibody 1.

In certain embodiments, the deglycosylation of the anti-CD2 antibody or antigen binding fragment thereof described herein can be determined by any appropriate method known in the art. Standard methods to determine deglycosylation include, but are not limited to, size-exclusion chromatography analysis, tertiary structure analysis, thermal stability evaluation, guanidine HCl induced denaturation, resistance to papain digestion, and reduced mass data of heavy chains measured by liquid chromatography-mass spectroscopy (LC-MS).

### (iv) Cells and Vectors

Provided herein are cells expressing the anti-CD2 antibody or antigen binding fragment thereof as described herein. Provided herein are ex vivo cells recombinantly expressing the anti-CD2 antibody or antigen binding fragment thereof as described herein. Also provided herein are expression vectors comprising nucleotide sequences that encode the anti-CD2 antibody or antigen binding fragment thereof as described herein. In a specific embodiment, the cell is an ex vivo cell.

In certain embodiments, the gene expression can be placed under the control of a promoter. The promoter can be a viral promoter, such as CMV or SV40; or a non-viral promoter such as elongation factor (EF)-1 promotor, UBC, PGK, or CAG promoter. In certain embodiments, a nucleotide sequence that encodes the anti-CD2 antibody or antigen binding fragment thereof described herein is cloned into an appropriate vector. In certain embodiments, the vector can be a mammalian vector. In certain embodiments, the vector can be a viral vector. In certain embodiments, the expression vector is transformed into a cell (e.g., an ex vivo cell). In certain embodiments, the cell expresses the vector such that the nucleotide sequence is transcribed and translated by the host cell. In certain embodiments, the host cell line can include bacteria, yeast, or mammalian cell lines. In a specific embodiment, the host cells line is a mammalian cell. In a certain embodiment, the mammalian cell lines that can be used as a host cell line for recombinant antibody production includes, but are not limited to, Chinese hamster ovary (CHO) cell line, NSO cell line, Sp2/0 cell line, PER.C6 cell line, and human embryonic kidney (HEK) cell line.

In certain embodiments, the host cell line can be genetically modified. In certain embodiments, the modification can be made with the goal of improved productivity of the expression of the recombinant protein. In certain embodiments, the modification can be made to act upon the recombinant protein the host cell expresses. In a specific embodiment, the host cell line has been genetically modified to express a desired glycosyltransferase. In a specific embodiment, the host cell line has been genetically modified to express EndoS or EndoS2.

### 7.1.1.3 Pharmaceutical Compositions

Provided herein are pharmaceutical compositions comprising a pharmaceutically effective amount of an anti-CD2 antibody or antigen binding fragment thereof as described herein. In certain embodiments, pharmaceutical compositions disclosed herein can have an anti-CD2 antibody or antigen binding fragment thereof as described herein and at least one pharmaceutically acceptable carrier. As used herein, the term “pharmaceutically acceptable carrier, excipient, or diluent” means a carrier, excipient, or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a therapeutic agent is administered. For example, the term pharmaceutically acceptable carrier, excipient or diluent includes any and all solvents, dispersion media, coatings, antibacterial and antifungal agents, isotonic and absorption delaying agents, and the like.

In certain embodiments, pharmaceutical compositions described herein can be formulated for subcutaneous, intravenous, intravascular, topical, intrarticular, intraarterial, intracranial, intramuscular, oral, intraorbital, inhalation, intraperitonial, intraosseous, endotracheal, sublingual, buccal, rectal, intradermal, intrathecal, intramedullary, or transdermal route of administration. In a specific embodiment, the composition comprising the anti-CD2 antibody or antigen binding fragment thereof described herein are formulated for intravenous administration. In a specific embodiment, the composition comprising the anti-CD2 antibody or antigen binding fragment thereof described herein are formulated for intrathecal administration.

In certain embodiments, the composition or formulation comprising the anti-CD2 antibody or antigen binding fragment thereof of the present disclosure comprises histidine HCl, sucrose, and/or polysorbate 80 (e.g., 10 mH histidine HCl, 234 mM sucrose, and 0.04% polysorbate 80).

In certain embodiments, the composition is formulated as a solution, suspension, or an emulsion. In certain embodiments, the composition is lyophilized. In certain embodiments, the anti-CD2 antibody or antigen binding fragment thereof described herein can be formulated for intravenous or intrathecal administration by combining the anti-CD2 antibody or antigen binding fragment thereof described herein with a pharmaceutically appropriate vehicle. In certain embodiments, vehicles used can be, but are not limited to, water, saline, Ringer's solution, dextrose solution, glycerol, ethanol, 1-10% human serum albumin, 5% dextrose in water, N-methylpyrrolidone, dimethyl sulfoxide, N,N-dimethylacetamide, propylene glycol, polyethylene glycol 400, diethylene glycol monoethyl ether, TWEEN 80, TWEEN 20, polyoxyl-35 castor oil, polyoxyl 40 hydrogenated castor oil, caprylocaproyl macrogol-8-glycerides, soybean oil, polyxoyethyllated oleic glycerides, and medium chain mono- and diglycerides. In certain embodiments, liposomes and non-aqueous vehicles such as fixed oils can also be used to administer an agent (e.g., an anti-CD2 antibody or antigen binding fragment thereof) as described herein.

In certain embodiments, the vehicle can contain additives that maintain isotonicity (e.g., sodium chloride, mannitol). In certain embodiments, the vehicle can contain additives to maintain chemical stability. These additives can include, but are not limited to, buffers (e.g., maleic acid, tartaric acid, lactic acid, citric acid, acetic acid, sodium bicarbonate, and sodium phosphate), and preservatives (e.g., detergents, oxidizing agents, and ionic buffers). The resulting pharmaceutical formulation is sterilized by known or suitable techniques.

In certain embodiments, the intravenous administration of the composition comprising the anti-CD2 antibody or antigen binding fragment thereof described herein can be administered as a bolus injection, a slow intravenous injection, or a continuous intravenous infusion. In certain embodiments, the intrathecal administration of the composition comprising the anti-CD2 antibody or antigen binding fragment thereof described herein can be administered as bolus doses via spinal catheter or lumbar puncture. In certain embodiments, the intrathecal administration of the composition comprising the anti-CD2 antibody or antigen binding fragment thereof described herein can be administered continuously into the intrathecal space by an implantable pump.

### 7.1.1.4 Kits

Provided herein are kits for use in practicing any of the methods disclosed herein. In certain embodiments, kits disclosed herein can contain the anti-CD2 antibody or antigen binding fragment thereof as described herein and at least one container. In certain embodiments, kits disclosed herein can contain the anti-CD2 antibody or antigen binding fragment thereof as described herein for use in in treating or preventing ALS when administered to a subject in need thereof.

In some embodiments, a kit includes one or more anti-CD2 antibody or an antigen binding fragment thereof and/or a second agent (e.g., acetaminophen, diphenhydramine, corticosteroids, antihistamines, epinephrine, bronchodilators, oxygen, nonsteroidal anti-inflammatory drug, oral analgesics, antihistamine, and/or immunosuppressant) of the present disclosure or a composition thereof in the same or a different container. In some embodiments, a kit includes at least one anti-CD2 antibody or an antigen binding fragment thereof in the same or a different container. In some embodiments, a kit contains a library of anti-CD2 antibody or an antigen binding fragment thereof. In some embodiments, a kit includes another active agent/second agent (e.g., acetaminophen, diphenhydramine, corticosteroids, antihistamines, epinephrine, bronchodilators, oxygen, nonsteroidal anti-inflammatory drug, oral analgesics, antihistamine, and/or immunosuppressant). In some embodiments, a kit includes a control and/or a reference (e.g., a reference antibody). In some embodiments, a kit may further include reagents and/or instructions for creating and/or synthesizing an anti-CD2 antibody or an antigen binding fragment thereof, compounds and/or compositions described herein. In some embodiments, a kit may also include one or more buffers. In some embodiments, a kit includes another agent in the same or a different container. In some embodiments, a container with an agent (e.g., anti-CD2 antibody or an antigen binding fragment thereof) is provided for a single dose administration or multiple dose administrations. In some embodiments, an agent (e.g., anti-CD2 antibody or an antigen binding fragment thereof) is present in a container in a kit in an amount sufficient for multiple dosages, usages, or administration. In some embodiments, a kit includes other components necessary for administration of an agent (e.g., anti-CD2 antibody or an antigen binding fragment thereof) (e.g., a kit includes a syringe, a catheter, a cannula, a pump, or any injection device). In certain embodiments, the kit comprises devices that can be used to administer the pharmaceutical composition described herein, including, but not limited to, syringes, needle-less injectors, drip bags, perfusion pumps, pumps, patches, and inhalers. In some embodiments, a kit includes a pharmaceutically acceptable carrier, diluent, excipient, and/or buffer, in the same or separate container as the container holding one or more of agent as described herein.

Components of a kit can be in separate containers or can be combined in a single container. In some embodiments, kit components may be packaged either in aqueous media, in powder form, in crystal form, or in lyophilized form. The containers that the pharmaceutical composition can be packaged in can include, but are not limited to, bottles, packets, ampoules, tubes, inhalers, bags, vials, and containers. The container means of the kits can include at least one of a vial, test tube, flask, bottle, needle-less injectors, drip bags, perfusion pumps, pumps, patches, an inhaler, ampoules, syringe or other container means, into which a component may be placed. In some embodiments, a component is suitably aliquoted. In some embodiments, where there is more than one kit component, a reagent and the corresponding label can be packaged together. In some embodiments, a kit contains second, third or other additional containers into which additional components may be separately placed. In some embodiments, a kit includes a second container means for containing sterile, pharmaceutically acceptable buffers and/or other diluents. In some embodiments, various combinations of components are included in one or more vial. In some embodiments, a kit includes a means for containing antibody and/or compounds and/or compositions of the present disclosure in close confinement for commercial sale. Such containers may include injection or blow-molded plastic containers into which desired vials are retained.

In some embodiments, kit components are provided in one and/or more liquid solutions. In some embodiments, a liquid solution is an aqueous solution or a sterile aqueous solution. In some embodiments, kit components are provided as a depyrogenized solution. In some embodiments, kit components can comprise an anti-CD2 antibody or an antigen binding fragment thereof in solution. In some embodiments, an anti-CD2 antibody or an antigen binding fragment thereof is provided in solution at a concentration of about or at least about 10 mg/mL, 15 mg/mL, 20 mg/mL, 25 mg/mL, 30 mg/mL, 35 mg/mL, 40 mg/mL, 45 mg/mL, 50 mg/mL, 10 mg/mL to 50 mg/mL, 15 mg/mL to 45 mg/mL, 25 mg/mL to 45 mg/mL, 35 mg/mL to 45 mg/mL, or 35 mg/mL to 40 mg/mL.

In some embodiments, kit components are provided as dried powder(s). When reagents and/or components are provided as dry powders, such powders may be reconstituted by the addition of suitable volumes of a solvent. In some embodiments, the solvents are provided in another container means. In some embodiments, kit components can comprise an anti-CD2 antibody or an antigen binding fragment thereof as dried powders. In some embodiments, the anti-CD2 antibody or an antigen binding fragment thereof can be re-suspended in any suitable solvent, such as sterile saline. In some embodiments, labeling dyes are provided as dried powders. In some embodiments, labeling dyes are provided in an amount of about, at least about, or at most about 10, 20, 30, 40, 50, 60, 70, 80, 90, 100, 120, 120, 130, 140, 150, 160, 170, 180, 190, 200, 300, 400, 500, 600, 700, 800, 900, 1000 micrograms. In some embodiments, dye can be re-suspended in any suitable solvent, such as DMSO.

In some embodiments, a kit can include instructions for using the components of the kit. In some embodiments, a kit contains instructions related to dosage, administration, applications, storage conditions, a list of diseases that can be treated or prevented by using one or more of the kit components, and/or use of the components. In some embodiments, instructions are recorded on a suitable recording medium. For example, instructions can be printed on a substrate, such as a paper, a tag (e.g., adhesive tag), or plastic. In some embodiments, instructions are present in a kit as a package insert or on a label attached to a container or components of a kit. In some embodiments, instructions are provided as electronic storage data file present on a suitable computer readable storage medium, e.g., CD-ROM, diskette, USB storage device, or flash drive. In some embodiments, instructions are not present in a kit but are present in a remote source, e.g., via the internet.

### 7.1.2 Patient Populations

Described herein are methods for treating or preventing ALS in a subject suspected of having or diagnosed with ALS. The term “subject” and “patient” are used interchangeably throughout the present disclosure. Subjects for whom the methods provided herein can be used are diagnosed and selected based on assessing diagnostic criteria and/or biomarkers associated with ALS according to the methods described in Section 7.3.1 and Section 7.3.2, respectfully. In certain embodiments, subjects for whom the methods provided herein can be used may have a particular stage of ALS at the time of treatment. Methods of determining disease stage are detailed in Section 7.3.3 below.

In certain embodiments, methods of the present disclosure are directed, in part, toward preventing ALS in a subject suspected of having or diagnosed with ALS. As used herein, the terms “prevention” and “preventing” refer to obtaining beneficial or desired results including, but not limited, to prophylactic benefit. For prophylactic benefit, the anti-CD2 antibodies or antigen binding fragments thereof and pharmaceutical compositions disclosed herein can be administered according to the methods of treating as provided herein to a subject at risk of developing ALS (e.g., a subject diagnosed as having possible ALS) or to a subject reporting one or more physiological symptoms of ALS, even though a diagnosis of the ALS may not have been made. In some embodiments, the subject has at least one symptom associated with ALS. Non-limiting examples of symptoms of ALS include dyspnea, orthopnea, muscle weakness, dysphagia, and dysarthria.

In certain embodiments, methods of the present disclosure are directed, in part, toward treating ALS in a subject suspected of having or diagnosed with ALS. As used herein and unless otherwise indicated, the terms “treat,” “treating,” “treatment,” and “ameliorating” are used interchangeably herein, and mean an alleviation, in whole or in part, of a disorder, disease or condition, such as ALS, or one or more of the symptoms associated with a disorder, disease, or condition, such as ALS, or slowing or halting of further progression or worsening of those symptoms, or alleviating or eradicating the cause(s) of the disorder, disease, or condition itself, such as ALS.

In certain embodiments, a subject to the methods disclosed herein is a mammal. In some embodiments, the subject is a human subject. In some embodiments, the subject is an adult subject. In some embodiments, the subject is female. In some embodiments, the subject is male. In some embodiments, the human subject is between about 18 to about 90 years of age. In some embodiments, the human subject is over 65, over 70, over 75, over 80, over 85, or over 90 years of age.

In certain embodiments, a subject to the methods disclosed herein has been diagnosed has having ALS according to Gold Coast criteria (see Section 7.3.1.3).

In certain embodiments, subjects in need of treatment disclosed herein can include subjects diagnosed as having possible, laboratory supported probable, probable, or definite ALS according to revised El Escorial Criteria (see Section 7.3.1.1). In some embodiments, a subject to the methods for treating or preventing ALS disclosed herein can be a subject diagnosed as having possible ALS. In some embodiments, a subject to the methods for treating or preventing ALS disclosed herein can be a subject diagnosed as having laboratory supported probable ALS. In some embodiments, a subject to the methods for treating or preventing ALS disclosed herein can be a subject diagnosed as having probable ALS. In some embodiments, a subject to the methods for treating or preventing ALS disclosed herein can be a subject diagnosed as having definite ALS.

In certain embodiments, subjects in need of treatment disclosed herein can include subjects diagnosed as having clinically definite, clinically probable, or clinically possible ALS according to Awaji shima criteria (See Section 7.3.1.2) In some embodiments, a subject to the methods for treating or preventing ALS disclosed herein can be a subject diagnosed as having clinically definite ALS. In some embodiments, a subject to the methods for treating or preventing ALS disclosed herein can be a subject diagnosed as having clinically probable ALS. In some embodiments, a subject to the methods for treating or preventing ALS disclosed herein can be a subject diagnosed as having clinically possible ALS.

In certain embodiments, the subject is a human subject recently diagnosed with ALS (e.g., diagnosed as having first symptoms within 3 months to 3 years from start of administration with an anti-CD2 antibody or antigen binding fragment thereof of the present disclosure). In some embodiments, the subject is a human subject diagnosed as having first symptoms within 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months from start of administration with an anti-CD2 antibody or antigen binding fragment thereof of the present disclosure. In some embodiments, the human subject is a naïve subject. In some embodiments, the human subject has not been treated for ALS. In some embodiments, the human subject has been previously treated with an agent that is used for treating ALS. In some embodiments, the human subject was not responsive to previous ALS treatment(s). In some embodiments, the subject is unresponsive to at least one treatment for ALS. In some embodiments, the subject is unresponsive to several treatments of ALS. In some embodiments, the subject has sporadic ALS. In some embodiments, the subject has familial ALS. In some embodiments, the subject has a genetic mutation associated with ALS (e.g., mutation in C9orf72, SOD1, TARDBP and/or FUS). In some embodiments, the subject does not have another acute or chronic disorder of the immune system (e.g., only ALS). In some embodiments, the subject does not have another immune related disorder (e.g., only ALS). In some embodiments, the subject does not have another chronic or acute inflammatory disorder of the immune system (e.g., only ALS). In some embodiments, the subject does not have another disease or condition besides ALS. In some embodiments, the subject has another acute or chronic disorder of the immune system (e.g., in addition to ALS). In some embodiments, the subject has another immune related disorder (e.g., in addition to ALS). In some embodiments, the subject has another chronic or acute inflammatory disorder of the immune system (e.g., in addition to ALS). In some embodiments, the subject has another disease or condition in addition to ALS.

In certain embodiments, patients for whom the methods provided herein can be used include ALS patients at various disease stages (see Section 7.3.3). In some embodiments, ALS patients for whom the methods provided herein can be used can be staged according to the King's system of staging. In some embodiments, ALS patients for whom the methods provided herein can be used are stage 1 ALS patients, stage 2 ALS patients, stage 3 ALS patients, and/or stage 4 ALS patients according to the King's system of staging. In some embodiments, an ALS patient for whom the methods provided herein can be used is a stage 1 ALS patient according to the King's system of staging. In some embodiments, an ALS patient for whom the methods provided herein can be used is a stage 2 ALS patient according to the King's system of staging. In some embodiments, an ALS patient for whom the methods provided herein can be used is a stage 3 ALS patient according to the King's system of staging. In some embodiments, an ALS patient for whom the methods provided herein can be used is a stage 4 ALS patient according to the King's system of staging. In some preferred embodiments, an ALS patient for whom the methods provided herein can be used is a stage 2 ALS patient according to the King's system of staging. In some other preferred embodiments, an ALS patient for whom the methods provided herein can be used is a stage 1 ALS patient according to the King's system of staging.

In some embodiments, ALS patients for whom the methods provided herein can be used can be staged according to the MiToS system of staging. In some embodiments, ALS patients for whom the methods provided herein can be used are stage 1 ALS patients, stage 2 ALS patients, stage 3 ALS patients, and/or stage 4 ALS patients according to the MiToS system of staging. In some embodiments, an ALS patient for whom the methods provided herein can be used is a stage 0 ALS patient according to the MiToS system of staging, wherein the patient reports one or more physiological symptoms of ALS. In some embodiments, an ALS patient for whom the methods provided herein can be used is a stage 1 ALS patient according to the MiToS system of staging. In some embodiments, an ALS patient for whom the methods provided herein can be used is a stage 2 ALS patient according to the MiToS system of staging. In some embodiments, an ALS patient for whom the methods provided herein can be used is a stage 3 ALS patient according to the MiToS system of staging. In some embodiments, an ALS patient for whom the methods provided herein can be used is a stage 4 ALS patient according to the MiToS system of staging. In some preferred embodiments, an ALS patient for whom the methods provided herein can be used is a stage 2 ALS patient according to the MiToS system of staging. In some other preferred embodiments, an ALS patient for whom the methods provided herein can be used is a stage 1 ALS patient according to the MiToSsystem of staging.

### 7.1.3 Dosages and Regimens

In certain embodiments, a dose of an anti-CD2 antibody or antigen binding fragment thereof is a flat dose and not dependent on body weight. In some embodiments, the anti-CD2 antibody or antigen binding fragment thereof is administered to a subject in need thereof at an amount ranging from about 0.1 mg to about 1 g, about 1 mg to about 1 g, or about 10 mg to about 1 g. In some embodiments, the anti-CD2 antibody or antigen binding fragment thereof is administered to a subject in need thereof at an amount of about 0.1 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1 g, or more than about 1 g. In some embodiments, the anti-CD2 antibody or antigen binding fragment thereof is administered to a subject in need thereof at an amount of at least about 0.1 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1 g, or more than about 1 g. In some embodiments, the anti-CD2 antibody or antigen binding fragment thereof is administered to a subject in need thereof at an amount of at most about 0.1 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1 g. In some embodiments, the anti-CD2 antibody or antigen binding fragment thereof is administered to a subject in need thereof at an amount of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1 g, or more than about 1 g. In some embodiments, the anti-CD2 antibody or antigen binding fragment thereof is administered to a subject in need thereof at an amount of at least about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1 g, or more than about 1 g. In some embodiments, the anti-CD2 antibody or antigen binding fragment thereof is administered to a subject in need thereof at an amount of at most about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, or about 1 g.

In certain embodiments, a dose of an anti-CD2 antibody or antigen binding fragment thereof is dependent on body weight (e.g., mg/kg). In certain embodiments, an anti-CD2 antibody or antigen binding fragment thereof is administered to a subject in order to treat or prevent ALS at a dose amount of 0.01 mg/kg/dose, 0.05 mg/kg/dose, 0.1 mg/kg/dose, 0.5 mg/kg/dose, 1.0 mg/kg/dose, 2.0 mg/kg/dose, 3.0 mg/kg/dose, 4.0 mg/kg/dose, 5.0 mg/kg/dose, 5.5 mg/kg/dose, 6.0 mg/kg/dose, 6.1 mg/kg/dose, 6.2 mg/kg/dose, 6.3 mg/kg/dose, 6.4 mg/kg/dose, 6.5 mg/kg/dose, 6.6 mg/kg/dose, 6.7 mg/kg/dose, 6.8 mg/kg/dose, 6.9 mg/kg/dose, 7.0 mg/kg/dose, 7.1 mg/kg/dose, 7.2 mg/kg/dose, 7.3 mg/kg/dose, 7.4 mg/kg/dose, 7.5 mg/kg/dose, 7.6 mg/kg/dose, 7.7 mg/kg/dose, 7.8 mg/kg/dose, 7.9 mg/kg/dose, 8.0 mg/kg/dose, 8.5 mg/kg/dose, 9.0 mg/kg/dose, 10 mg/kg/dose, 11 mg/kg/dose, 12 mg/kg/dose, 13 mg/kg/dose, 14 mg/kg/dose, 15 mg/kg/dose, 16 mg/kg/dose, 17 mg/kg/dose, 18 mg/kg/dose, 19 mg/kg/dose, 20 mg/kg/dose, 21 mg/kg/dose, 22 mg/kg/dose, 23 mg/kg/dose, 24 mg/kg/dose, 25 mg/kg/dose, 26 mg/kg/dose, 27 mg/kg/dose, 28 mg/kg/dose, 29 mg/kg/dose, 30 mg/kg/dose, 31 mg/kg/dose, 32 mg/kg/dose, 33 mg/kg/dose, 34 mg/kg/dose, 35 mg/kg/dose, 36 mg/kg/dose, 37 mg/kg/dose, 38 mg/kg/dose, 39 mg/kg/dose, 40 mg/kg/dose, 41 mg/kg/dose, 42 mg/kg/dose, 43 mg/kg/dose, 44 mg/kg/dose, 45 mg/kg/dose, 46 mg/kg/dose, 47 mg/kg/dose, 48 mg/kg/dose, 49 mg/kg/dose, 50 mg/kg/dose, 51 mg/kg/dose, 52 mg/kg/dose, 53 mg/kg/dose, 54 mg/kg/dose, 55 mg/kg/dose, 56 mg/kg/dose, 57 mg/kg/dose, 58 mg/kg/dose, 59 mg/kg/dose, 60 mg/kg/dose, 61 mg/kg/dose, 62 mg/kg/dose, 63 mg/kg/dose, 64 mg/kg/dose, 65 mg/kg/dose, 66 mg/kg/dose, 67 mg/kg/dose, 68 mg/kg/dose, 69 mg/kg/dose, 70 mg/kg/dose, 71 mg/kg/dose, 72 mg/kg/dose, 73 mg/kg/dose, 74 mg/kg/dose, 75 mg/kg/dose, 76 mg/kg/dose, 77 mg/kg/dose, 78 mg/kg/dose, 79 mg/kg/dose, 80 mg/kg/dose, 81 mg/kg/dose, 82 mg/kg/dose, 83 mg/kg/dose, 84 mg/kg/dose, 85 mg/kg/dose, 86 mg/kg/dose, 87 mg/kg/dose, 88 mg/kg/dose, 89 mg/kg/dose, 90 mg/kg/dose, 91 mg/kg/dose, 92 mg/kg/dose, 93 mg/kg/dose, 94 mg/kg/dose, 95 mg/kg/dose, 96 mg/kg/dose, 97 mg/kg/dose, 98 mg/kg/dose, 99 mg/kg/dose, or 100 mg/kg/dose.

In certain embodiments, an anti-CD2 antibody or antigen binding fragment thereof described herein can be administered to patient in order to treat or prevent ALS at dose ranges of between 0.01 mg/kg/dose and 50 mg/kg/dose, between 0.5 mg/kg/dose and 40 mg/kg/dose, between 1.0 mg/kg/dose and 30 mg/kg/dose, between 1.5 mg/kg/dose and 20 mg/kg/dose, between 2.0 mg/kg/dose and 10 mg/kg/dose, between 2.5 mg/kg/dose and 9.5 mg/kg/dose, between 3.0 mg/kg/dose and 9.0 mg/kg/dose, between 3.5 mg/kg/dose and 8.5 mg/kg/dose, between 4.0 mg/kg/dose and 7.0 mg/kg/dose, or between 4.0 mg/kg/dose and 10.0 mg/kg/dose. In a specific embodiment, an anti-CD2 antibody or antigen binding fragment thereof described herein can be administered to a patient in order to treat or prevent ALS at a dose amount of about 7.0 mg/kg/dose. In a specific embodiment, an anti-CD2 antibody or antigen binding fragment thereof described herein can be administered to a patient in order to treat or prevent ALS at a dose amount of about 10.0 mg/kg/dose. In a specific embodiment, an anti-CD2 antibody or antigen binding fragment thereof described herein can be intravenously administered to a patient in order to treat or prevent ALS at a dose amount of 7.0 mg/kg/dose. In a specific embodiment, an anti-CD2 antibody or antigen binding fragment thereof described herein can be intravenously administered to a patient in order to treat or prevent ALS at a dose amount of 10.0 mg/kg/dose.

In certain embodiments, an anti-CD2 antibody or antigen binding fragment thereof is administered to a subject in order to treat or prevent ALS at a dose amount sufficient to achieve a steady state of the anti-CD2 antibody or an antigen binding fragment thereof in the subject. In certain embodiments, an anti-CD2 antibody or antigen binding fragment thereof is administered to a subject in order to treat or prevent ALS at a dose amount sufficient to achieve a peripheral anti-CD2 antibody or antigen binding fragment thereof trough (C0) concentration. Peripheral anti-CD2 antibody or antigen binding fragment thereof trough (C0) concentration can be measured in serum from blood samples collected

from subjects according to methods described herein (e.g., see paragraph [00228]). In some embodiments, an anti-CD2 antibody or antigen binding fragment thereof is administered to a subject in order to treat or prevent ALS at a dose amount sufficient to achieve a peripheral anti-CD2 antibody or antigen binding fragment thereof trough (C0) concentration greater than about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, or more than 25 μg/mL. In some embodiments, an anti-CD2 antibody or antigen binding fragment thereof is administered to a subject in order to treat or prevent ALS at a dose amount sufficient to achieve a peripheral anti-CD2 antibody or antigen binding fragment thereof trough (C0) concentration of about or at least about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, or more than 25 μg/mL. In some embodiments, an anti-CD2 antibody or antigen binding fragment thereof is administered to a subject in order to treat or prevent ALS at a dose amount sufficient to achieve a peripheral anti-CD2 antibody or antigen binding fragment thereof trough (C0) concentration of about or at least about 1 μg/mL to 25 μg/mL, 2 μg/mL to 24 μg/mL, 3 μg/mL to 23 μg/mL, 4 μg/mL to 22 μg/mL, 5 μg/mL to 21 μg/mL, 6 μg/mL to 20 μg/mL, 7 μg/mL to 19 μg/mL, 8 μg/mL to 18 μg/mL, 9 μg/mL to 17 μg/mL, 15 μg/mL to 25 μg/mL, 16 μg/mL to 24 μg/mL, 17 μg/mL to 23 μg/mL, 18 μg/mL to 22 μg/mL, 18 μg/mL to 21 μg/mL, 18 μg/mL to 20 μg/mL, or 19 μg/mL.

In certain embodiments, an anti-CD2 antibody or antigen binding fragment thereof is administered to a subject in order to treat or prevent ALS at a dose amount sufficient to achieve a steady state of the anti-CD2 antibody or an antigen binding fragment thereof in the subject's CSF. Typically, following repeated administration of an agent (e.g., an anti-CD2 antibody or antigen binding fragment thereof), a steady-state is reached when the quantity of the agent eliminated from the CSF in the unit of time equals the quantity of the agent that reaches the CSF in the unit of time. In some embodiments, an anti-CD2 antibody or antigen binding fragment thereof is administered to a subject in order to treat or prevent ALS at a dose amount sufficient to achieve a steady-state amount of anti-CD2 antibody or an antigen binding fragment thereof in the subject's CSF that is at least about 0.5 ng/ml, at least about 1 ng/ml, at least about 2 ng/ml, at least about 3 ng/ml, at least about 5 ng/ml, at least about 6 ng/ml, at least about 7 ng/ml, at least about 8 ng/ml, at least about 9 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 75 ng/ml, at least about 80 ng/ml, at least about 85 ng/ml, at least about 90 ng/ml, at least about 95 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 ng/ml, at least about 900 ng/ml, at least about 1000 ng/ml, or more than about 1000 ng/ml. In some embodiments, an anti-CD2 antibody or antigen binding fragment thereof is administered to a subject in order to treat or prevent ALS at a dose amount sufficient to achieve a steady-state amount of anti-CD2 antibody or an antigen binding fragment thereof in the subject's CSF ranging from about 0.5 ng/ml to about 100 ng/ml, about 1 ng/ml to about 90 ng/ml, about 5 ng/ml to about 80 ng/ml, about 10 ng/ml to about 70 ng/ml, or about 15 ng/ml to about 60 ng/ml. In some embodiments, an anti-CD2 antibody or antigen binding fragment thereof is administered to a subject in order to treat or prevent ALS at a dose amount sufficient to achieve a steady-state amount of 0.5 ng/ml, 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 26 ng/ml, 27 ng/ml, 28 ng/ml, 29 ng/ml, 30 ng/ml, 31 ng/ml, 32 ng/ml, 33 ng/ml, 34 ng/ml, 35 ng/ml, 36 ng/ml, 37 ng/ml, 38 ng/ml, 39 ng/ml, 40 ng/ml, 41 ng/ml, 42 ng/ml, 43 ng/ml, 44 ng/ml, 45 ng/ml, 46 ng/ml, 47 ng/ml, 48 ng/ml, 49 ng/ml, 50 ng/ml, 51 ng/ml, 52 ng/ml, 53 ng/ml, 54 ng/ml, 55 ng/ml, 56 ng/ml, 57 ng/ml, 58 ng/ml, 59 ng/ml, 60 ng/ml, 61 ng/ml, 62 ng/ml, 63 ng/ml, 64 ng/ml, 65 ng/ml, 70 ng/ml, 80 ng/ml, or 90 ng/ml an anti-CD2 antibody or antigen binding fragment thereof in the subject's CSF.

In some embodiments, a steady state of the anti-CD2 antibody or an antigen binding fragment thereof in the subject's CSF can be achieved after the first dose, after the second dose, after the third dose, after the fourth dose, or after the fifth dose. In some embodiments, a steady state of the anti-CD2 antibody or an antigen binding fragment thereof in the subject's CSF can be achieved after the first dose, after the second dose, after the third dose, or after the fourth dose. In some embodiments, a steady state of the anti-CD2 antibody or an antigen binding fragment thereof in the subject's CSF can be achieved after the first dose, after the second dose, or after the third dose. In some embodiments, a steady state of the anti-CD2 antibody or an antigen binding fragment thereof in the subject's CSF can be achieved at least after 1 hours, at least after 2 hours, at least after 3 hours, at least after 4 hours, at least after 5 hours, at least after 6 hours, at least after 7 hours, at least after 8 hours, at least after 9 hours, at least after 10 hours, at least after 11 hours, or at least after 12 hours after administering a dose of an anti-CD2 antibody or an antigen binding fragment thereof. In some embodiments, a steady state of the anti-CD2 antibody or an antigen binding fragment thereof in the subject's CSF can be achieved within 1 hour to 48 hours, 2 hours to 36 hours, 3 hours to 24 hours, or 4 hours to 24 hours after administering a dose of the anti-CD2 antibody or an antigen binding fragment thereof.

In some embodiments, a steady state of the anti-CD2 antibody or an antigen binding fragment thereof in the subject's CSF can be for about 1 day to about 120 days, about 1 day to about 110 days, about 1 day to about 100 days, about 1 day to about 90 days, about 1 day to about 70 days, about 1 day to about 60 days, about 1 day to about 50 days, about 1 day to about 40 days, or about 1 days to about 30 days. In some embodiments, a steady state of the anti-CD2 antibody or an antigen binding fragment thereof in the subject's CSF can be for about 1 day, 7 days, 14 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or more than 120 days. In some embodiments, a steady state of the anti-CD2 antibody or an antigen binding fragment thereof in the subject's CSF can be for about 1 to 30 days, about 1 to 50 days, about 1 to 90 days, about 1 to 110 days, about 30 to 50 days, about 30 to 90 days, about 30 to 110 days, about 50 to 90 days, about 50 to 110 days, or about 30 to 50 days.

In some embodiments, an anti-CD2 antibody or antigen binding fragment thereof described herein is administered to a subject weekly, four times in a month, biweekly, daily, or monthly. In some embodiments, an anti-CD2 antibody or antigen binding fragment thereof described herein is administered to a subject weekly. In some embodiments, an anti-CD2 antibody or antigen binding fragment thereof described herein is administered to a subject once every 1-2 weeks, 2-3 weeks, 3-4 weeks, 4-5 weeks, 5-6 weeks, 6-7 weeks, 7-8 weeks, 9-10 weeks, 10-11 weeks, 11-12 weeks, 12-13 weeks, 13-14 weeks, 14-15 weeks, 15-16 weeks, 16-17 weeks, 17-18 weeks, 18-19 weeks, 19-20 weeks, or more than every 20 weeks. In some embodiments, an anti-CD2 antibody or antigen binding fragment thereof described herein is administered to a subject once every 3-3.5 months, every 3.5-4 months, every 4-4.5 months, every 4.5-5 months, every 5-5.5 months, every 5.5-6 months, or more than every 6 months. In some embodiments, an anti-CD2 antibody or antigen binding fragment thereof described herein is administered to a subject once every week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 4.5 months, once every 5 months, once every 5.5 months, or once every 6 months. In some embodiments, an anti-CD2 antibody or antigen binding fragment thereof described herein is administered to a subject every 4 weeks. In some embodiments, an anti-CD2 antibody or antigen binding fragment thereof described herein is administered to a subject every 14 weeks. In some embodiments, an anti-CD2 antibody or antigen binding fragment thereof described herein is administered to a subject every 16 weeks. In some embodiments, a second dose of an anti-CD2 antibody or antigen binding fragment thereof described herein is administered to a subject about 2-5 weeks after the first dose. In some embodiments, a second dose of an anti-CD2 antibody or antigen binding fragment thereof described herein is administered to a subject about 4 weeks after the first dose. In some embodiments, a third dose of an anti-CD2 antibody or antigen binding fragment thereof described herein is administered to a subject about 2-5 weeks, or 4 weeks after the second dose. In some embodiments, a fourth dose of an anti-CD2 antibody or antigen binding fragment thereof described herein is administered to a subject about 2-5 weeks, or 4 weeks after the third dose. In some embodiments, a fourth dose of an anti-CD2 antibody or antigen binding fragment thereof described herein is administered to a subject about 10-20 weeks, or 16 weeks after the third dose. In some embodiments, a fifth dose of an anti-CD2 antibody or antigen binding fragment thereof described herein is administered to a subject about 13-15 weeks, or 14 weeks after the fourth dose. In some embodiments, a subsequent dose of an anti-CD2 antibody or antigen binding fragment thereof described herein is administered to a subject about 4-26 weeks after a previous dose.

In some embodiments, an anti-CD2 antibody or antigen binding fragment thereof described herein is administered to a subject for a period of time to treat or ameliorate ALS in the subject. In some embodiments, an anti-CD2 antibody or antigen binding fragment thereof described herein is administered to a subject for about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 6 months, 8 months, 9 months, 10 months, 11 months, 12 months, 1.5 years, 2 years, or longer than 2 years.

In some embodiments, an anti-CD2 antibody or antigen binding fragment thereof described herein is administered to a subject every 4 weeks up to week 12 and once 14 weeks thereafter. In some embodiments, an anti-CD2 antibody or antigen binding fragment thereof described herein is administered in one treatment regimen, where the treatment regimen comprises administering 4 doses to a subject every 4 weeks up to week 12 and one dose 14 weeks thereafter. In some embodiments, the administering of an anti-CD2 antibody or antigen binding fragment thereof described herein occurs three times between the first administration and the last administration for a total of five doses administered to a subject. In some embodiments, the last administration of an anti-CD2 antibody or antigen binding fragment thereof described herein is 26 weeks after the first administration.

In some embodiments, an anti-CD2 antibody or antigen binding fragment thereof described herein is administered to a subject every 4 weeks up to week 8 and once 16 weeks thereafter. In some embodiments, an anti-CD2 antibody or antigen binding fragment thereof described herein is administered in one treatment regimen, where the treatment regimen comprises administering 3 doses to a subject every 4 weeks up to week 8 and one dose 16 weeks thereafter. In some embodiments, the administering of an anti-CD2 antibody or antigen binding fragment thereof described herein occurs two times between the first administration and the last administration for a total of four doses administered to a subject. In some embodiments, the last administration of an anti-CD2 antibody or antigen binding fragment thereof described herein is 24 weeks after the first administration.

In some embodiments, at least one treatment regimen can be administered to a subject described herein. In some embodiments, more than one treatment regimen can be administered to a subject described herein. Where more than one treatment regimen is to be administered to the subject, a subsequent treatment regimen can be administered at 52 weeks after the first dose of the previously administered treatment regimen (i.e., 26 weeks or 28 weeks after the last dose of the previously administered treatment regimen). In some embodiments, multiple rounds of treatment regimens can be administered to a subject described herein for the duration of the subject's life span or until the subject acquires toxicity or resistance to the anti-CD2 antibody or antigen binding fragment thereof described herein.

In certain embodiments, the anti-CD2 antibody or antigen binding fragment thereof described herein can be formulated for subcutaneous, intravenous, intravascular, topical, intrarticular, intraarterial, intracranial, intramuscular, oral, intraorbital, intravitreal, inhalation, intraperitonial, intraosseous, endotracheal, sublingual, buccal, rectal, intradermal, intrathecal, intramedullary, or transdermal routes of administration. One of skill in the art would appreciate that the dosage of the anti-CD2 antibody or antigen binding fragment thereof described herein can be varied depending on the route of administration. In a specific embodiment, the anti-CD2 antibody or antigen binding fragment thereof described herein is formulated for intravenous administration to a patient to treat or ALS. In a specific embodiment, the anti-CD2 antibody or antigen binding fragment thereof described herein is formulated for intrathecal administration to a patient with ALS.

In certain embodiments, the patient with ALS can be administered the pharmaceutical composition comprising an effective amount of the anti-CD2 antibody or antigen binding fragment thereof described herein. In certain embodiments, the pharmaceutical composition comprising an effective amount of the anti-CD2 antibody or antigen binding fragment thereof described herein is contained in a kit or system for administration to a patient in order to treat or prevent ALS. Details of pharmaceutical compositions and kits suitable for use with the methods disclosed herein are found in Sections 7.1.1.3 and 7.1.1.4, respectively.

### 7.1.4 Combination Regimens

In certain embodiments, methods provided herein can comprise administering to the human subject the anti-CD2 antibody or antigen binding fragment thereof of the present disclosure or a pharmaceutical composition comprising the anti-CD2 antibody or antigen binding fragment thereof of the present disclosure in combination with one or more agents. Provided herein are methods of administering an anti-CD2 antibody or antigen binding fragment thereof in combination with another agent(s) that treats or prevents ALS (Section 7.1.4.1). Also provided herein are methods of administering an anti-CD2 antibody or antigen binding fragment thereof in combination with an agent(s) that ameliorates or prevents a side effect associated with treatment or administration of the anti-CD2 antibody or antigen binding fragment thereof of the present disclosure (Section 7.1.4.2).

### 7.1.4.1 Combination Regimens with Agents for Treating ALS

Provided herein are methods of administering an anti-CD2 antibody or antigen binding fragment thereof in combination with another agent that treats or prevents ALS (e.g., based on the agent's product label or known to one skilled in the art e.g., Mead R J et al., Nat Rev Drug Discov. 2022 Dec. 21:1-28). In certain embodiments, an agent to be used in combination with an anti-CD2 antibody or antigen binding fragment thereof disclosed herein can be an FDA approved drug indicated for the treatment of ALS. As known in the art at the time of filing, these agents include: AMX0035 (a 3:1 combination of sodium phenylbutyrate (4-phenyl butyric acid sodium salt) and taurursodiol (2-[(3a, 7β-dihydroxy-24-oxo-5ß-cholan-24-yl)amino]ethane sulfonic acid, dihydrate) also known as tauroursodeoxycholic acid or TUDCA); edaravone (3-methyl-1-phenyl-2-pyrazolin-5-one]; and riluzole (2-amino-6-(trifluoromethoxy)benzothiazole).

In certain embodiments, an agent to be used in combination with an anti-CD2 antibody or antigen binding fragment thereof disclosed herein can be a drug under clinical trial for the treatment of ALS. Non-limiting examples of such agents include Tocilizumab, ACTH/Acthar gel, IL-2, Ibudilast, Pentoxifylline, Masitinib, NP001, Thalidomide, Glatiramer Acetate, CC100, Fingolimod, Minocycline, Pioglitazone, DNL747, Fasudil, Ono-2506, Anakinra, RNS60, Retigabine, Memantine, Talampanel, Perampanel, Mexiletine, Ceftriaxone, Celecoxib, Lamotrigine, Nimodipine, Gabapentin, Epi-589, N-Acetylcysteine, Vitamine E, Coenzyme Q10, Selegiline, Rasagiline, Cu(II)-ATSM, BIIB067, Pyrimethamine, Levosimendan, Tirasemtiv, CK-2127107IGF-1, Xaliproden, EPO, BDNF, GM604, CNTF, SB509, VM202, sNN0029, KP-100, Dexpramipexole, Olexosime, Creatine, Acetyl-L-carnitine, Lithium, Rapamycin, Tamoxifen, Ozanezumab, GDC-0134, YAM80, Methylcobalamin, Pimozide, Omigapil/TCH346, Deferiprone, Arimoclomol, EH301, BIIB078, and MD1003 (see e.g., Wobst H J et al., Med Res Rev. 2020 July; 40 (4): 1352-1384).

In certain embodiments, an agent to be used in combination with an anti-CD2 antibody or antigen binding fragment thereof disclosed herein can be a drug not known to slow progression of the disease but is used to control one or more symptoms of ALS. Non-limiting examples of agents used to control symptoms of ALS include topiramate (2,3:4,5-Bis-O-(1-methylethylidene)-beta-D-fructopyranose sulfamate); gabapentin (2-[1-(aminomethyl)cyclohexyl]acetic acid); lamotrigine (6-(2,3-dichlorophenyl)-1,2,4-triazine-3,5-diamine); talampanel ((8R)-7-Acetyl-5-(4-aminophenyl)-8,9-dihydro-8-methyl-7H-1,3-dioxolo[4,5-h][2,3]benzodiazepine); ceftriaxone ((6R,7R)-7-[[(2Z)-2-(2-amino-1,3-thiazol-4-yl)-2-methoxyiminoacetyl]amino]-3-[(2-methyl-5,6-dioxo-1H-1,2,4-triazin-3-yl) sulfanylmethyl]-8-ox 0-5-thia-1-azabicyclo[4.2.0]oct-2-ene-2-carboxylic acid); an inhibitor of glutamate carboxypeptidase II; dextromethorphan/quinidine; TUDCA (tauroursodeoxycholic acid); baclofen (4-amino-3-(4-chlorophenyl) butanoic acid); diazepam (7-chloro-1-methyl-5-phenyl-3H-1,4-benzodiazepin-2-one); trihexyphenidyl (1-cyclohexyl-1-phenyl-3-piperidin-1-ylpropan-1-ol); and, amitriptyline (N,N-dimethyl-3-(2-tricyclo[9.4.0.03,8]pentadeca-1 (15),3,5,7,11,13-hexaenylidene) propan-1-amine). In some embodiments, the methods provided herein comprise administering to the subject a drug that has been repurposed for the treatment of ALS (e.g., edaravone, rasagiline, AMX0035, masitinib, and/or acrimonclomol). See also Kiernan et al., Nat Rev Neurol. 2021 February; 17 (2): 104-118.

In certain embodiments, an agent to be used in combination with an anti-CD2 antibody or antigen binding fragment thereof disclosed herein can be an off-label drug used to treat ALS. Off-label use of a drug is usually described as any use of a drug outside its approved indications. Non-limiting examples of off-label drugs used to treat ALS include abacavir, dolutegravir, lamivudine, curcumin, sodium phenylbutyrate, naltrexone, tauroursodeoxycholic acid (TUDCA), methylcobalamin, dextromethorphan hydrobromide, quinidine sulfate, nudexta, ropinirol, metformin, tamoxifen, trimetazidine, masitinib, CuATSM (diacetylbis(N (4)-methylthiosemicarbazonato) copper (II)), reldesemtiv, combinations thereof, and the like. See also Pridmore W, (2021). RRNMF Neuromuscular Journal, 2 (1), 36-40 and the ALSUntangled Group website. In certain embodiments, an off-label drug used to treat ALS suitable for use with the methods provided herein can be sodium phenylbutyrate, TUDCA, or a combination thereof.

In some embodiments, the methods provided herein comprise administering to the subject (a) an effective amount of an anti-CD2 antibody or antigen binding fragment thereof described herein and (b) an effective amount of another agent that treats or prevents ALS or a symptom thereof. In a specific embodiment, an anti-CD2 antibody or antigen binding fragment thereof described herein can be administered in combination with sodium phenylbutyrate, TUDCA, or a combination thereof. In another specific embodiment, an anti-CD2 antibody or antigen binding fragment thereof described herein can be administered in combination with riluzole.

In certain embodiments, an anti-CD2 antibody or antigen binding fragment thereof described herein can be administered to a subject who is on or has been on a dosing regimen comprising an agent that treats or prevents ALS. In some embodiments, an anti-CD2 antibody or antigen binding fragment thereof described herein can be administered to a subject who is on or has been on a dosing regimen comprising sodium phenylbutyrate. In some embodiments, an anti-CD2 antibody or antigen binding fragment thereof described herein can be administered to a subject who is on or has been on a dosing regimen comprising TUDCA. In some embodiments, an anti-CD2 antibody or antigen binding fragment thereof described herein can be administered to a subject who is on or has been on a dosing regimen comprising sodium phenylbutyrate and TUDCA. In some embodiments, an anti-CD2 antibody or antigen binding fragment thereof described herein can be administered to a subject who is on or has been on a dosing regimen comprising riluzole. In some embodiments, an anti-CD2 antibody or antigen binding fragment thereof described herein can be administered to a subject who has been on a stable dose of sodium phenylbutyrate, TUDCA, riluzole, or any combination thereof for about 1 month to about one year, about 1 month to about 6 months, or about 1 month to about 3 months. In some embodiments, an anti-CD2 antibody or antigen binding fragment thereof described herein can be administered to a subject who has been on a stable dose of sodium phenylbutyrate, TUDCA, riluzole, or any combination thereof for at least 20, 21, 22, 23, 24, 25, 26, 27, 28, 29, 30, 21, 32, 33, 34, or 35 days.

In certain embodiments, a dosing regimen disclosed herein comprising administering of an anti-CD2 antibody or antigen binding fragment thereof to a subject can coincide with a dosing regimen comprising an agent that treats or prevents ALS. In certain embodiments, a dosing regimen disclosed herein comprising administering of an anti-CD2 antibody or antigen binding fragment thereof to a subject can coincide with a sodium phenylbutyrate, TUDCA, riluzole, or any combination thereof dosing regimen. In some embodiments, an anti-CD2 antibody or antigen binding fragment thereof can be administered to a subject at any of the dosing schedules disclosed herein while the subject is also being administered a daily dose of sodium phenylbutyrate. In some embodiments, an anti-CD2 antibody or antigen binding fragment thereof can be administered to a subject at any of the dosing schedules disclosed herein while the subject is also being administered a daily dose of TUDCA. In some embodiments, an anti-CD2 antibody or antigen binding fragment thereof can be administered to a subject at any of the dosing schedules disclosed herein while the subject is also being administered a daily dose of sodium phenylbutyrate and TUDCA. In some embodiments, an anti-CD2 antibody or antigen binding fragment thereof can be administered to a subject at any of the dosing schedules disclosed herein while the subject is also being administered a daily dose of riluzole. In some embodiments, an anti-CD2 antibody or antigen binding fragment thereof can be administered to a subject once every 4 to 14 weeks while the subject is also being administered a daily dose of sodium phenylbutyrate. In some embodiments, an anti-CD2 antibody or antigen binding fragment thereof can be administered to a subject once every 4 to 14 weeks while the subject is also being administered a daily dose of TUDCA. In some embodiments, an anti-CD2 antibody or antigen binding fragment thereof can be administered to a subject once every 4 to 14 weeks while the subject is also being administered a daily dose of sodium phenylbutyrate and TUDCA. In some embodiments, an anti-CD2 antibody or antigen binding fragment thereof can be administered to a subject once every 4 to 14 weeks while the subject is also being administered a daily dose of riluzole. In some embodiments, an anti-CD2 antibody or antigen binding fragment thereof can be administered to a subject once every 4 to 14 weeks while the subject is also being administered a daily dose of riluzole and TUDCA. In some embodiments, an anti-CD2 antibody or antigen binding fragment thereof can be administered to a subject once every 4 to 14 weeks while the subject is also being administered a daily dose of riluzole and sodium phenylbutyrate. In some embodiments, an anti-CD2 antibody or antigen binding fragment thereof can be administered to a subject once every 4 to 14 weeks while the subject is also being administered a daily dose of riluzole, TUDCA, and sodium phenylbutyrate.

In some embodiments, an effective amount of an anti-CD2 antibody or antigen binding fragment thereof can be administered to a subject while the subject is also being administered a daily dose of about 50 mg to about 150 mg of riluzole. In some embodiments, an effective amount of an anti-CD2 antibody or antigen binding fragment thereof can be administered to a subject while the subject is also being administered a daily dose of 100 mg of riluzole.

In some embodiments, an effective amount of an anti-CD2 antibody or antigen binding fragment thereof can be administered to a subject while the subject is also being administered a daily dose of about 1 g to about 3 g of TUDCA. In some embodiments, an effective amount of an anti-CD2 antibody or antigen binding fragment thereof can be administered to a subject while the subject is also being administered a daily dose of 1 g, 2 g, or 3 g of TUDCA. In some embodiments, an effective amount of an anti-CD2 antibody or antigen binding fragment thereof can be administered to a subject while the subject is also being administered a daily dose of about 3 g to about 6 g of sodium phenylbutyrate. In some embodiments, an effective amount of an anti-CD2 antibody or antigen binding fragment thereof can be administered to a subject while the subject is also being administered a daily dose of 3 g, 4 g, 5 g, or 6 g of sodium phenylbutyrate. In some embodiments, an effective amount of an anti-CD2 antibody or antigen binding fragment thereof can be administered to a subject while the subject is also being administered a daily dose of about 1 g to about 3 g of TUDCA and a daily dose of about 3 g to about 6 g of sodium phenylbutyrate. In some embodiments, an effective amount of an anti-CD2 antibody or antigen binding fragment thereof can be administered to a subject while the subject is also being administered a daily dose of TUDCA and sodium phenylbutyrate where the ratio of TUDCA to sodium phenylbutyrate administered is 1:1, 1:2, or 1:3. In certain embodiments, an effective amount of an anti-CD2 antibody or antigen binding fragment thereof can be administered to a subject while the subject is also being administered a daily dose of TUDCA and sodium phenylbutyrate in a 3:1 ratio. In some specific embodiments, an effective amount of an anti-CD2 antibody or antigen binding fragment thereof can be administered to a subject while the subject is also being administered a daily dose of 1 g TUDCA and 3 g sodium phenylbutyrate. In other specific embodiments, an effective amount of an anti-CD2 antibody or antigen binding fragment thereof can be administered to a subject while the subject is also being administered a daily dose of 2 g TUDCA and 6 g sodium phenylbutyrate.

In some embodiments, an effective amount of an anti-CD2 antibody or antigen binding fragment thereof can be administered to a subject while the subject is also being administered a daily dose of about 50 mg to about 150 mg of riluzole and a daily dose of about 1 g to about 3 g of TUDCA. In some embodiments, an effective amount of an anti-CD2 antibody or antigen binding fragment thereof can be administered to a subject while the subject is also being administered a daily dose of about 50 mg to about 150 mg of riluzole and a daily dose of about 3 g to about 6 g of sodium phenylbutyrate. In some embodiments, an effective amount of an anti-CD2 antibody or antigen binding fragment thereof can be administered to a subject while the subject is also being administered a daily dose of about 50 mg to about 150 mg of riluzole, a daily dose of about 3 g to about 6 g of sodium phenylbutyrate, and a daily dose of about 1 g to about 3 g of TUDCA. In certain embodiments, an effective amount of an anti-CD2 antibody or antigen binding fragment thereof can be administered to a subject while the subject is also being administered a daily dose of 100 mg of riluzole and a daily dose of TUDCA and sodium phenylbutyrate in a 3:1 ratio. In certain embodiments, an effective amount of an anti-CD2 antibody or antigen binding fragment thereof can be administered to a subject while the subject is also being administered a daily dose of 100 mg of riluzole and a daily dose of 1 g TUDCA and 3 g sodium phenylbutyrate. In certain embodiments, an effective amount of an anti-CD2 antibody or antigen binding fragment thereof can be administered to a subject while the subject is also being administered a daily dose of 100 mg of riluzole and a daily dose of 2 g TUDCA and 6 g sodium phenylbutyrate.

In some embodiments, an effective amount of an anti-CD2 antibody or antigen binding fragment thereof can be administered to a subject while the subject is also being administered a standard of care ALS treatment. Non-limiting examples of standard of care ALS treatments include mechanical ventilation, tracheostomy, physical therapy, acupuncture, speech/swallowing therapy, nutritional support, and the like.

### 7.1.4.2 Combination Regimens with Agents for Treating Side Effects

In some embodiments, an anti-CD2 antibody or antigen binding fragment thereof of the present disclosure can be used in combination with an agent that ameliorates or prevents a side effect associated with treatment or administration of the anti-CD2 antibody or antigen binding fragment thereof of the present disclosure (e.g., corticosteroids, antihistamines, epinephrine, bronchodilators, oxygen, diphenhydramine, NSAIDs, and/or oral analgesics/antipyretics (e.g., paracetamol/acetaminophen)). In some embodiments, prior to the administration of an anti-CD2 antibody or antigen binding fragment thereof about 650-1000 mg acetaminophen (i.e., paracetamol) and/or an H1-antagonist (antihistamine, e.g., 25 mg diphenhydramine or 4 mg chlorpheniramine) is administered to a subject, for example to minimize a symptom or side effect of an injection site. In some embodiments, prednisone is administered to a subject. In some embodiments, diphenhydramine (e.g., 25 mg oral) is administered to a subject, for example, about 15 minutes prior to administration (e.g., intrathecal, intravenous, and/or subcutaneous administration) of an anti-CD2 antibody or antigen binding fragment thereof. In some embodiments, acetaminophen (e.g., 15 mg/kg oral; up to 650 mg) is administered to a subject, for example, about 15 minutes prior to administration (e.g., intrathecal, intravenous, and/or subcutaneous administration) of an anti-CD2 antibody or antigen binding fragment thereof.

In some embodiments, an additional agent is an agent that ameliorates at least one side effect associated with ALS or treatment with an anti-CD2 antibody or antigen binding fragment thereof of the present disclosure is administered to a subject. In some embodiments, a steroid is administered to a subject. In some embodiments, a steroid is administered prior to or after an anti-CD2 antibody or antigen binding fragment thereof of the present disclosure. In some embodiments, the steroid is administered in an amount between about 5 mg and about 30 mg. In some embodiments, the steroid is methylprednisolone (e.g., about, at least about, or at most about 250 mg IV). In some embodiments, the steroid described herein is administered in an amount between about 5 mg and about 250 mg. In some embodiments, the steroid is administered in an amount between about 5 mg and about 150 mg. In some embodiments, the steroid is administered in an amount between about 8 mg and about 120 mg. In some embodiments, the steroid is administered in an amount between about 10 mg and about 200 mg. In some embodiments, the steroid is administered in an amount of about 10 mg. In some embodiments, the steroid is administered in an amount of 10 mg. In some embodiments, the steroid is administered in an amount between about 18 mg and about 22 mg. In some embodiments, the steroid is administered in an amount of about 20 mg. In some embodiments, the steroid is administered in an amount of 20 mg. In some embodiments, the steroid is dexamethasone. In some embodiments, the steroid is corticosteroid. In some embodiments, the steroid is dexamethasone or corticosteroid and is administered in an amount between about 10 mg and about 20 mg. In some embodiments, the steroid is dexamethasone or corticosteroid and is administered in an amount of about 10 mg. In some embodiments, the steroid is dexamethasone. In some embodiments, dexamethasone or corticosteroid is administered intravenously. In some embodiments, the steroid is dexamethasone or corticosteroid and is administered in an amount of about 20 mg. In some embodiments, the steroid is dexamethasone or corticosteroid and is administered between about 15 minutes and 75 minutes before each administration of an anti-CD2 antibody or antigen binding fragment thereof of the present disclosure. In some embodiments, the steroid is dexamethasone or corticosteroid and is administered about or at most about 60 minutes before each administration of an anti-CD2 antibody or antigen binding fragment thereof of the present disclosure. In some embodiments, the steroid is dexamethasone or corticosteroid and is administered about 60 minutes before an administration of an anti-CD2 antibody or antigen binding fragment thereof of the present disclosure.

In some embodiments, acetaminophen (e.g., 650 mg orally) or an equivalent is administered to a subject. In some embodiments, an antipyretic is administered to a subject. In some embodiments, an analgesic is administered to a subject. In some embodiments, diphenhydramine (e.g., 25 to 50 mg intravenously or orally) or an equivalent is administered to a subject for treating rash, pruritus, or other signs and/or symptoms of hypersensitivity (allergic) reaction. In some embodiments, pressors, fluids, oxygen, epinephrine, bronchodilators, ventilatory support, antipyretic, and/or analgesic is administered to a subject.

In some embodiments, an antihistamine is administered prior to or after an anti-CD2 antibody or antigen binding fragment thereof of the present disclosure. In some embodiments, the antihistamine is an H1 antagonist. In some embodiments, the H1 antagonist is a first generation H1 antagonist. In some embodiments, the antihistamine is an ethanolamine. In some embodiments, the ethanolamine is diphenhydramine, carbinoxamine, doxylamine, orphenadrine, bromazine, clemastine, dimenhydrinate, or any combination thereof. In some embodiments, the antihistamine is diphenhydramine. In some embodiments, the antihistamine is administered in an amount between about 20 mg and 60 mg. In some embodiments, the antihistamine is administered in an amount between about 20 mg and 30 mg. In some embodiments, the antihistamine is administered in an amount of about 25 mg. In some embodiments, the antihistamine is administered in an amount of 25 mg. In some embodiments, the antihistamine is administered in an amount between about 40 mg and 60 mg. In some embodiments, the antihistamine is administered in an amount between about 45 mg and 55 mg. In some embodiments, the antihistamine is administered in an amount of about 50 mg. In some embodiments, the antihistamine is administered in an amount of 50 mg. In some embodiments, the antihistamine is diphenhydramine and the amount between about 20 mg and about 30 mg. In some embodiments, the antihistamine is diphenhydramine and the amount is about 25 mg. In some embodiments, the antihistamine is diphenhydramine and is administered between about 10 minutes and 70 minutes before each administration of an anti-CD2 antibody or antigen binding fragment thereof of the present disclosure. In some embodiments, the antihistamine is diphenhydramine and is administered between about 5 minutes and 60 minutes before each administration of an anti-CD2 antibody or antigen binding fragment thereof of the present disclosure. In some embodiments, the antihistamine is diphenhydramine and is administered between about 15 minutes and 60 minutes before an anti-CD2 antibody or antigen binding fragment thereof of the present disclosure. In some embodiments, about 25 mg of diphenhydramine is administered between about 15 minutes and 30 minutes before an administration of an anti-CD2 antibody or antigen binding fragment thereof of the present disclosure. In some embodiments, about 25 mg of diphenhydramine is administered between about 5 minutes and 20 minutes before an administration of an anti-CD2 antibody or antigen binding fragment thereof of the present disclosure.

In some embodiments, acetaminophen is administered in an amount between about 100 mg and 1000 mg. In some embodiments, acetaminophen is administered in an amount between about 400 mg and 600 mg. In some embodiments, acetaminophen is administered in an amount of about 500 mg. In some embodiments, acetaminophen is administered in an amount of 500 mg. In some embodiments, acetaminophen is administered in an amount between about 500 mg and 800 mg. In some embodiments, acetaminophen is administered in an amount between about 550 mg and 750 mg. In some embodiments, acetaminophen is administered in an amount between about 600 mg and 700 mg. In some embodiments, acetaminophen is administered in an amount of about 650 mg. In some embodiments, acetaminophen is administered in an amount of 650 mg. In some embodiments, the acetaminophen is administered between about 15 minutes and about 45 minutes before each administration of an anti-CD2 antibody or antigen binding fragment thereof of the present disclosure. In some embodiments, the acetaminophen is administered about 30 minutes before each administration of an anti-CD2 antibody or antigen binding fragment thereof of the present disclosure. In some embodiments, the acetaminophen is administered between about 10 minutes and about 60 minutes before an administration of an anti-CD2 antibody or antigen binding fragment thereof of the present disclosure. In some embodiments, about 650 mg of acetaminophen is administered about 15 minutes before an administration of an anti-CD2 antibody or antigen binding fragment thereof of the present disclosure.

In some embodiments, a steroid, an H1 antagonist, and acetaminophen are administered prior to and/or after an anti-CD2 antibody or antigen binding fragment thereof of the present disclosure. In some embodiments, diphenhydramine is administered in an amount of about 25 mg to 50 mg, and/or acetaminophen is administered in an amount of about 650 mg prior to and/or after an anti-CD2 antibody or antigen binding fragment thereof of the present disclosure. In some embodiments, chlorpheniramine (e.g., 4 mg) is administered prior to and/or after an anti-CD2 antibody or antigen binding fragment thereof of the present disclosure is administered to a subject (e.g., between 30 minutes and 3 hours prior to administration of the anti-CD2 antibody or antigen binding fragment thereof).

### 7.2 Outcome Assessment

As provided herein, an anti-CD2 antibody or antigen binding fragment thereof of the present disclosure can be used in a method for treating, preventing or ameliorating at least one symptom associated with amyotrophic lateral sclerosis (ALS) in a subject in need thereof, such as a method or use described in Section 7.1.3. Various systems for determining the stage of ALS progression are known in the art and include the King's system of staging and the MitoS system of staging such as those described in Section 7.3.3.

In certain embodiments, the effect of a method of administering an anti-CD2 antibody or antigen binding fragment thereof as provided herein can be assessed by monitoring clinical signs and symptoms of the disease (e.g., ALS) to be treated. In certain embodiments, the effect of a method of administering an anti-CD2 antibody or antigen binding fragment thereof as provided herein can be assessed by monitoring cell type markers in biological samples (e.g., blood, serum, PBMCs, CSF) collected from the subject receiving the anti-CD2 antibody or antigen binding fragment thereof (see Section 7.3.2). The efficacy of a method of administering an anti-CD2 antibody or antigen binding fragment thereof described herein may be assessed at about 1 week, about 2 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, at about 2-3 months, about 3-4 months, about 4-5 months, about 5-6 months, about 6-7 months, about 7-8 months, about 8-9 months, about 9-10 months, about 10-11 months, about 11-12 months, about 12-18 months, about 18-24 months, about 24-36 months, about 1-2 years, about 2-3 years, about 3-4 years, about 4-5 years, about 5-6 years, about 6-7 years, about 7-8 years, about 8-9 years, or about 9-10 years after initiation of treatment in accordance with the methods described herein.

In certain embodiments, methods of administering an anti-CD2 antibody or antigen binding fragment thereof provided herein can result in a change in the Amyotrophic Lateral Sclerosis Functional Rating Scale-revised (ALSFRS-R) score compared to baseline. In the context of an assessment of the effect of a method of treatment as disclosed herein, the term “baseline” refers to a measurement pre-treatment (e.g., to the ALSFRS-R total score obtained from the subject prior to the administering of the anti-CD2 antibody). The ALSFRS-R score assesses the progression in ALS disease severity and can define a specific rate at which subject ALS disease severity is increasing. ALSFRS-R is a score from 0-48 assessing disability. The ALSFRS-R includes 12 questions, each being rated on a five-point scale from 0=cannot do, to 4=normal ability. Individual item scores are summed to produce a reported score of between 0=worst and 48-best. See Cedarbaum J M, J Neurol Sci 1999; 169:13-21. The term “progression of ALSFRS-R score” means the rate of change in ALSFRS-R score prior to treatment initiation expressed in points per unit of time (e.g., weeks or months). As the disease progresses, the ALSFRS-R score decreases, i.e., the rate of change in ALSFRS-R score is a loss of points.

In some embodiments, methods of administering an anti-CD2 antibody or antigen binding fragment thereof provided herein can maintain the subject's ALSFRS-R total score compared to baseline. In some embodiments, the ALSFRS-R score increases in a subject treated in accordance with a method provided herein compared to baseline, indicating an improvement of symptoms. In some embodiments, methods of administering an anti-CD2 antibody or antigen binding fragment thereof provided herein can increase the subjects's ALSFRS-R total score by at least about 0.3, 0.4, 0.5, 0.6, 0.8, 0.9, 1.0, 1.1, 1.2, 1.3, 1.4, or 1.5 points compared to baseline. In some embodiments, methods of administering an anti-CD2 antibody or antigen binding fragment thereof provided herein can decrease the subject's ALSFRS-R total score by no more than about 0.3, 0.4, 0.5, 0.6, 0.8, 0.9, 1.0, 1.1, 1.2, 1.3, 1.4, or 1.5 points compared to baseline. In other embodiments, the ALSFRS-R score remains unchanged in a subject treated in accordance with a method provided herein compared to baseline.

“Progression of ALSFRS-R score” (point/month) can be evaluated from the date of first ALS-related symptom to time of first treatment (baseline). In other words, in certain embodiments, progression of ALSFRS-R score before treatment initiation corresponds to the rate of change of ALSFRS-R score from the date of first ALS-related symptom to time of first treatment (baseline). In some embodiments, methods of administering an anti-CD2 antibody or antigen binding fragment thereof provided herein can slow the subject's disease progression rate by at least 0.5, 0.6, 0.7, 0.8, 0.9, 1.0, or 1.1 ALSFRS-R points per month (ALSFRS-R/month) compared to baseline. In some embodiments, methods of administering an anti-CD2 antibody or antigen binding fragment thereof provided herein can slow the subject's disease progression rate by about 0.5 to 1.5, about 0.8 to about 1.1, or by 0.5, 0.6, 0.7, 0.8, 0.9, 1.0, or 1.1 ALSFRS-R points per month (ALSFRS-R/month) compared to baseline.

In certain embodiments, methods of administering an effective amount of an anti-CD2 antibody or antigen binding fragment thereof provided herein can result in an increased life expectancy of the subject after the administering as compared to a subject having a similar diagnosis and rate of disease progression that was not administered the effective amount of an anti-CD2 antibody or an antigen binding fragment thereof. In some embodiments, the life expectancy can be increased by about a month, 3 months, 6 months, a year, 2 years, 3 years, 4 years, 5 years, or greater than 5 years in a subject treated in accordance with a method provided herein compared to a subject having a similar diagnosis and rate of disease progression that was not administered the effective amount of an anti-CD2 antibody or an antigen binding fragment thereof. In some embodiments, the life expectancy remains unchanged in a subject treated in accordance with a method provided herein compared to a subject having a similar diagnosis and rate of disease progression that was not administered the effective amount of an anti-CD2 antibody or an antigen binding fragment thereof.

In certain embodiments, methods of administering an anti-CD2 antibody or antigen binding fragment thereof provided herein maintains or improves respiratory function of the subject compared to baseline. Respiratory function can be assessed by one or more tests such as forced vital capacity (FVC) and slow vital capacity (SVC). FVC (percent of predicted normal) is the vital capacity (VC) measured when the subject is exhaling with maximal speed and effort whereas SVC is the is the vital capacity (VC) measured when the subject is exhaling with minimal speed and effort. FVC and SVC can be measured using conventional spirometers that have had a calibration check prior to testing.

In some embodiments, methods of administering an anti-CD2 antibody or antigen binding fragment thereof provided herein results in a change in the subject's FVC compared to baseline. In some embodiments, methods of administering an anti-CD2 antibody or antigen binding fragment thereof provided herein can increase or maintain the subject's FVC compared to baseline. In some embodiments, methods of administering an anti-CD2 antibody or antigen binding fragment thereof provided herein can maintain the subject's FVC at 75% or higher, 80% or higher, or 85% or higher. In some embodiments, methods of administering an anti-CD2 antibody or antigen binding fragment thereof provided herein can increase the subject's FVC to 75% or higher, 80% or higher, or 85% or higher. In some embodiments, methods of administering an anti-CD2 antibody or antigen binding fragment thereof provided herein can reduce the decline of FVC in the subject by about 0.5% to about 15%, by about 1% to about 10%, or about by about 2% to about 5% compared to baseline and/or to a subject having a similar diagnosis and rate of disease progression that was not administered the effective amount of an anti-CD2 antibody or an antigen binding fragment thereof.

In some embodiments, methods of administering an anti-CD2 antibody or antigen binding fragment thereof provided herein results in a change in the subject's SVC compared to baseline. In some embodiments, methods of administering an anti-CD2 antibody or antigen binding fragment thereof provided herein can increase or maintain the subject's SVC compared to baseline. In some embodiments, methods of administering an anti-CD2 antibody or antigen binding fragment thereof provided herein can maintain the subject's SVC at 75% or higher, 80% or higher, or 85% or higher. In some embodiments, methods of administering an anti-CD2 antibody or antigen binding fragment thereof provided herein can increase the subject's SVC to 75% or higher, 80% or higher, or 85% or higher. In some embodiments, methods of administering an anti-CD2 antibody or antigen binding fragment thereof provided herein can reduce the decline of SVC in the subject by about 0.5% to about 15%, by about 1% to about 10%, or about by about 2% to about 5% compared to baseline and/or to a subject having a similar diagnosis and rate of disease progression that was not administered the effective amount of an anti-CD2 antibody or an antigen binding fragment thereof.

In some embodiments, methods of administering an anti-CD2 antibody or antigen binding fragment thereof provided herein results in a change in the subject's ALS stage compared to baseline. Various ALS staging systems are known in the art. Exemplary staging systems include the King's system and the MiToS system as described in Section 7.3.3.

In certain embodiments, improvement as a result of the methods provided herein can be a regression of the ALS stage according to the King's system (“King's ALS stage”) of the patient, such as going from stage 4 to stage 3, 2, or 1, from stage 3 to stage 2 or 1, and/or from stage 2 to stage 1, or from any stage of King's ALS stage to free of ALS. Such improvement as a result of the methods provided herein can also be a retardation of the progression of the patient's King's ALS stage, such as less rapid progression from one stage to another when compared with a similar patient not treated or treated with a placebo, or a stabilization of the patient's King's ALS stage, such as the patient's King's ALS staying at stage 1, 2, 3, or 4 without worsening of the disease into the next stage. In certain embodiments of the methods provided herein, the dose of the administered anti-CD2 antibody or antigen binding fragment thereof can be adjusted based on the therapeutic outcome as determined by the patient's King's ALS stage before and after the application of the method. In certain embodiments, if there is a regression and/or stabilization of the patient's King's ALS stage as described in this paragraph, then the treatment method for the patient is continued with the same or decreased dose of the administered anti-CD2 antibody or antigen binding fragment thereof. In certain embodiments, the dose of the anti-CD2 antibody or antigen binding fragment thereof is increased if there is no regression and/or no stabilization of the patient's King's ALS stage as described in this paragraph. In a specific embodiment, the method comprises (1) administering to the patient at a first dose of an anti-CD2 antibody or antigen binding fragment thereof provided herein, (2) determining the King's ALS stage of a patient, and (3) if the King's ALS stage of the patient is improved (e.g., regressed or stabilized), then administering the anti-CD2 antibody or antigen binding fragment thereof to the patient at the first dose or at a dose lower than the first dose; or, if the King's ALS stage of the patient is not improved, then administering the anti-CD2 antibody or antigen binding fragment thereof at a dose higher than the first dose.

In certain embodiments, improvement as a result of the methods provided herein can be a regression of the ALS stage according to the MiToS system (“MiTOS ALS stage”) of the patient, such as going from stage 4 to stage 3, 2, 1, or 0, from stage 3 to stage 2, 1, or 0, from stage 2 to stage 1 or 0, and/or from stage 1 to 0, or from any stage of MiToS ALS stage to free of ALS. Such improvement as a result of the methods provided herein can also be a retardation of the progression of the patient's MiToS ALS stage, such as less rapid progression from one stage to another when compared with a similar patient not treated or treated with a placebo, or a stabilization of the patient's MiToS ALS stage, such as the patient's MiToS ALS staying at stage 0, 1, 2, 3, or 4 without worsening of the disease into the next stage. In certain embodiments of the methods provided herein, the dose of the administered anti-CD2 antibody or antigen binding fragment thereof can be adjusted based on the therapeutic outcome as determined by the patient's MiTOS ALS stage before and after the application of the method. In certain embodiments, if there is a regression and/or stabilization of the patient's MiToS ALS stage as described in this paragraph, then the treatment method for the patient is continued with the same or decreased dose of the administered anti-CD2 antibody or antigen binding fragment thereof. In certain embodiments, the dose of the anti-CD2 antibody or antigen binding fragment thereof is increased if there is no regression and/or no stabilization of the patient's MiTOS ALS stage as described in this paragraph. In a specific embodiment, the method comprises (1) administering to the patient at a first dose of an anti-CD2 antibody or antigen binding fragment thereof provided herein, (2) determining the MiToS ALS stage of a patient, and (3) if the MiToS ALS stage of the patient is improved (e.g., regressed or stabilized), then administering the anti-CD2 antibody or antigen binding fragment thereof to the patient at the first dose or at a dose lower than the first dose; or, if the MiToS ALS stage of the patient is not improved, then administering the anti-CD2 antibody or antigen binding fragment thereof at a dose higher than the first dose.

Methods provided in the present disclosure herein include administering to the human subject the anti-CD2 antibody or antigen binding fragment thereof of the present disclosure or a pharmaceutical composition comprising the anti-CD2 antibody or antigen binding fragment thereof to treat and/or alleviate at least one symptom associated with ALS (e.g., dyspnea, orthopnea, muscle weakness, dysphagia, dysarthria). In certain embodiments, methods provided herein can modulate immunomodulatory activity associated with ALS in the treated subject.

Immunomodulatory activities exhibited by the anti-CD2 antibody or antigen binding fragment thereof described herein can include a decrease in pathogenic lymphocytes. Typically, in subjects having ALS, pathogenic lymphocytes are triggered in the periphery to infiltrate the central nervous system (CNS) and cause local inflammation and neurodegeneration. In some embodiments, the anti-CD2 antibody or antigen binding fragment thereof described herein can decrease circulating pathogenic lymphocytes, pathogenic lymphocytes located in the cerebrospinal fluid (CSF), or both.

Pathogenic lymphocytes include T cells, B cells, natural killer (NK) cells, or any combination thereof. T cells (CD3+ cells) are a class of lymphocytes, having specific T cell receptors (TCRs) that are produced as a result of gene rearrangement. T cells can be identified in subclasses, which include but are not limited to helper T cells (CD4+ T cells), killer T cells (CD8+ T cells), and regulatory T cells, or Tregs (CD4+/CD25+ T cells). CD4+T cell are mediators of cellular immunity that play a critical role in the activation of other immune cells such as B cells and CD8+ T cells, as well as in the regulation of immune responses. Tregs (CD4+/CD25+ T cells) are potent inhibitors of immune responses. B cells (CD19+ cells), also known as B lymphocytes, produce high-affinity antibodies, generate immunological memory, act as antigen-presenting cells, and secrete cytokines. Unlike other the lymphocytes (e.g., T cells and B cells), NK cells (CD3/CD16+CD56 cells) can selectively lyse cells in an antigen-independent manner that generally does not give rise to immunological memory or long-term protective immunity.

The determination of pathogenic lymphocytes can be achieved by any appropriate method known in the art. These methods can include, but are not limited to, cell proliferation assays, T cell activation functional assays, ELISPOT assays, intracellular staining, cytokine capture, tetramer staining, spectra-typing assays, flow cytometry, FACS (fluorescence activated cell sorting), single-cell RNA sequencing (scRNA-seq), and biosensor assays. In certain embodiments, blood, serum, PBMCs and/or CSF samples collected from a subject described herein can be subjected to scRNA-seq to determine percentages of lymphocytes subsets. In certain embodiments, PBMCs and/or CSF samples collected from a subject described herein can be subjected to flow cytometry to determine percentages of lymphocytes subsets based on cell surface markers as follows: T (CD3+) lymphocytes, B (CD19+) lymphocytes, helper/inducer T (CD3+CD4+) lymphocytes, suppressor/cytotoxic T (CD3+CD8+) lymphocytes, and natural killer (NK) (CD3-CD16+ and/or CD56+) lymphocytes. The helper/suppressor T-lymphocyte ratio (CD3+CD4+/CD3+CD8+) can also be determined using the same flow cytometry method.

Without being bound by any particular theory, any of the methods disclosed herein of administering the anti-CD2 antibody or antigen binding fragment thereof can decrease the level of T cells in the subject. In certain embodiments, methods of administering the anti-CD2 antibody or antigen binding fragment thereof described herein can decrease circulating T cells in the blood, cerebrospinal fluid (CSF), or both in the subject relative to circulating T cells in the blood and/or CSF without treatment with the anti-CD2 antibody or antigen binding fragment thereof (i.e., relative to the subject's baseline and/or relative to a subject having a similar diagnosis and rate of disease progression that was not administered the effective amount of an anti-CD2 antibody or an antigen binding fragment thereof). In some embodiments, methods of administering the anti-CD2 antibody or antigen binding fragment thereof described herein can decrease circulating T cells, T cells located in the cerebrospinal fluid (CSF), or both in the subject by at least about 50%, 55%, 60%, 65%, 70%, 75%, 80%, 85%, 90%, 95%, or 100% relative to circulating T cells in the blood and/or CSF without treatment with the anti-CD2 antibody or antigen binding fragment thereof. In some embodiments, methods of administering the anti-CD2 antibody or antigen binding fragment thereof described herein can decrease circulating T cells, T cells located in the cerebrospinal fluid (CSF), or both in the subject by about 50%, 55%, 60%, 65%, 70%, 75%, 80%, 85%, 90%, 95%, or 100% relative to circulating T cells in the blood and/or CSF without treatment with the anti-CD2 antibody or antigen binding fragment thereof. In some embodiments, methods of administering the anti-CD2 antibody or antigen binding fragment thereof described herein can decrease circulating T cells, T cells located in the cerebrospinal fluid (CSF), or both in the subject by 80%, 81%, 82%, 83%, 84%, 85%, 86%, 87%, 88%, 89%, 89%, 90%, 91%, 92%, 93%, 94%, 95%, 96%, 97%, 98%, 99%, 99%, or 100% relative to circulating T cells in the blood and/or CSF without treatment with the anti-CD2 antibody or antigen binding fragment thereof.

Without being bound by any particular theory, any of the methods disclosed herein of the anti-CD2 antibody or antigen binding fragment thereof can decrease the level of B cells in the subject. In certain embodiments, methods of administering the anti-CD2 antibody or antigen binding fragment thereof described herein can decrease circulating B cells, B cells located in the cerebrospinal fluid (CSF), or both in the subject relative to circulating B cells in the blood and/or CSF without treatment with the anti-CD2 antibody or antigen binding fragment thereof. In some embodiments, methods of administering the anti-CD2 antibody or antigen binding fragment thereof described herein can decrease circulating B cells, B cells located in the cerebrospinal fluid (CSF), or both in the subject by at least about 50%, 55%, 60%, 65%, 70%, 75%, 80%, 85%, 90%, 95%, or 100% relative to circulating B cells in the blood and/or CSF without treatment with the anti-CD2 antibody or antigen binding fragment thereof. In some embodiments, methods of administering the anti-CD2 antibody or antigen binding fragment thereof described herein can decrease circulating B cells, B cells located in the cerebrospinal fluid (CSF), or both in the subject by about 50%, 55%, 60%, 65%, 70%, 75%, 80%, 85%, 90%, 95%, or 100% relative to circulating B cells in the blood and/or CSF without treatment with the anti-CD2 antibody or antigen binding fragment thereof.

Without being bound by any particular theory, any of the methods disclosed herein of administering the anti-CD2 antibody or antigen binding fragment thereof can decrease the level of NK cells in the subject. In certain embodiments, methods of administering the anti-CD2 antibody or antigen binding fragment thereof described herein can decrease circulating NK cells, NK cells located in the cerebrospinal fluid (CSF), or both in the subject relative to circulating NK cells in the blood and/or CSF without treatment with the anti-CD2 antibody or antigen binding fragment thereof. In some embodiments, methods of administering the anti-CD2 antibody or antigen binding fragment thereof described herein can decrease circulating NK cells, NK cells located in the cerebrospinal fluid (CSF), or both in the subject by at least about 50%, 55%, 60%, 65%, 70%, 75%, 80%, 85%, 90%, 95%, or 100% relative to circulating NK cells in the blood and/or CSF without treatment with the anti-CD2 antibody or antigen binding fragment thereof. In some embodiments, methods of administering the anti-CD2 antibody or antigen binding fragment thereof described herein can decrease circulating NK cells, NK cells located in the cerebrospinal fluid (CSF), or both in the subject by about 50%, 55%, 60%, 65%, 70%, 75%, 80%, 85%, 90%, 95%, or 100% relative to circulating NK cells in the blood and/or CSF without treatment with the anti-CD2 antibody or antigen binding fragment thereof. In some embodiments, methods of administering the anti-CD2 antibody or antigen binding fragment thereof described herein can decrease circulating NK cells, NK cells located in the cerebrospinal fluid (CSF), or both in the subject by 75%, 76%, 77%, 78%, 79%, 80%, 81%, 82%, 83%, 84%, 85%, 86%, 87%, 88%, 89%, 89%, 90%, 91%, 92%, 93%, 94%, or 95% relative to circulating NK cells in the blood and/or CSF without treatment with the anti-CD2 antibody or antigen binding fragment thereof.

Without being bound by any particular theory, any of the methods disclosed herein of administering the anti-CD2 antibody or antigen binding fragment thereof can maintain or increase the level of Tregs in the subject. In certain embodiments, methods of administering the anti-CD2 antibody or antigen binding fragment thereof described herein can maintain the amount of circulating Tregs, Tregs located in the cerebrospinal fluid (CSF), or both relative to circulating Tregs in the blood and/or CSF without treatment with the anti-CD2 antibody or antigen binding fragment thereof. In some embodiments, methods of administering the anti-CD2 antibody or antigen binding fragment thereof described herein can increase circulating Tregs, Tregs located in the cerebrospinal fluid (CSF), or both by no more than about 25%, 20%, 15%, 10%, 5%, or 1% relative to circulating Tregs in the blood and/or CSF without treatment with the anti-CD2 antibody or antigen binding fragment thereof. In some embodiments, methods of administering the anti-CD2 antibody or antigen binding fragment thereof described herein can increase circulating Tregs, Tregs located in the cerebrospinal fluid (CSF), or both by at least about 1%, 5%, 10%, 15%, 20%, 25%, 50%, 75%, or 100% relative to circulating Tregs in the blood and/or CSF without treatment with the anti-CD2 antibody or antigen binding fragment thereof. In some embodiments, methods of administering the anti-CD2 antibody or antigen binding fragment thereof described herein can increase circulating Tregs, Tregs located in the cerebrospinal fluid (CSF), or both by about 1%, 5%, 10%, 15%, 20%, 25%, 50%, 75%, or 100% relative to circulating Tregs in the blood and/or CSF without treatment with the anti-CD2 antibody or antigen binding fragment thereof.

In certain embodiments, any of the methods disclosed herein of administering the anti-CD2 antibody or antigen binding fragment thereof can increase the percentage of the ratio of regulatory T cells to effector T cells (Treg:Teff) in the subject's blood and/or CSF relative to the Treg:Teff ratio in the blood and/or CSF without treatment with the anti-CD2 antibody or antigen binding fragment thereof. Any of the methods of measuring Treg:Teff known in the art can be used to measure Treg:Teff. Typically, Treg:Teff is measured by FACS sorting CD4+ T cells based on surface expression of CD25 and CD127 to yield: 1) CD4+CD127 lo/2 CD25+ T cells; and 2) CD4+CD127+CD25 2 T effector, or “Teff” cells. Following isolation, Teff cells are labeled with CFSE (C₂₉H₁₉NO₁₁; CAS No. 150347-59-4) and Treg cells are labeled with PKH26 (C₅₉H₉₇IN₂; CAS No. 154214-55-8). Fluorescence levels of the labeled Treg and Teff cells are measured, and the ratio is determined from the measurements. In some embodiments, methods of administering the anti-CD2 antibody or antigen binding fragment thereof described herein can increase Treg:Teff in the cerebrospinal fluid (CSF), or both by at least about 1%, 5%, 10%, 15%, 20%, 25%, 50%, 75%, or 100% relative to the Treg:Teff in the blood and/or CSF without treatment with the anti-CD2 antibody or antigen binding fragment thereof. In some embodiments, methods of administering the anti-CD2 antibody or antigen binding fragment thereof described herein can increase Treg:Teff in the cerebrospinal fluid (CSF), or both by about 1%, 5%, 10%, 15%, 20%, 25%, 50%, 75%, or 100% relative to the Treg:Teff in the blood and/or CSF without treatment with the anti-CD2 antibody or antigen binding fragment thereof.

In some embodiments, administration of or treatment with an anti-CD2 antibody or antigen binding fragment thereof of the present disclosure results in an increase in the ratio of CD4 Treg/Tem in a biological sample from the subject (e.g., in the subject's blood and/or CSF) as compared to a reference, baseline, or prior to administration or treatment. In some embodiments, Treg is defined as CD3+CD56-CD8-CD4+CD127loFoxP3+. In some embodiments, CD4+ Tem is defined as CD3+CD56-CD8-CD4+ Foxp3-CD45RA-CD45RO+CCR7−. In some embodiments, the ratio of CD4 Treg (CD3+CD56-CD8-CD4+CD127loFoxP3+)/Tem (CD3+CD56-CD8-CD4+ Foxp3-CD45RA-CD45RO+CCR7−) is measured via a multi-color flow cytometry. In some embodiments, treatment or administration of an anti-CD2 antibody or antigen binding fragment thereof of the present disclosure results in about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100% increase from a reference in the CD4 Treg/Tem ratio in a biological sample from a subject in need thereof (e.g., subject with ALS). In some embodiments, treatment with or administration of an anti-CD2 antibody or antigen binding fragment thereof of the present disclosure results in about a 75% increase or greater from baseline or a reference in the CD4 Treg/Tem ratio in a biological sample from a subject in need thereof (e.g., subject with ALS).

In some embodiments, a subject experiences a decrease in intensity, frequency, or experiences fewer adverse events after treatment with an anti-CD2 antibody or antigen binding fragment thereof as compared to prior to treatment. Examples of adverse events include systemic allergic reaction, lymphopenia, thrombocytopenia, neutropenia, anemia, asymptomatic hepatic injury, infection, and/or any adverse event grade 3 or higher. In some embodiments, an adverse event is a systemic allergic reaction, lymphopenia, thrombocytopenia, neutropenia, anemia, asymptomatic hepatic injury, infection, or any adverse event grade 3 or higher. In some embodiments, a subject has a decrease in frequency, intensity, or has fewer adverse events selected from any one or more of systemic allergic reaction, lymphopenia, thrombocytopenia, neutropenia, anemia, asymptomatic hepatic injury, infection, and/or any adverse event grade 3 or higher. In some embodiments, a subject is responsive to treatment with an anti-CD2 antibody or antigen binding fragment thereof of the present disclosure if the subject has a decrease in frequency, intensity, or has fewer adverse events after the start of treatment with an anti-CD2 antibody or antigen binding fragment thereof (e.g., from different time points after the start of treatment and/or at the end of treatment) as compared to prior to the start of treatment. In some embodiments, an adverse event is determined weekly, monthly, biweekly, daily, or at any suitable time.

### 7.3 Diagnostic Criteria, Biomarkers, and Disease Staging

Provided herein are various methods for selecting patients for treatment with the methods disclosed herein. Also provided herein are methods of selecting patients with various stages of ALS to be treated with the methods disclosed herein. Methods of diagnosing subjects to be treated with a method disclosed herein can include assessing diagnostic criteria (Section 7.3.1) and/or biomarkers associated with ALS (Section 7.3.2) in the subject. Methods of determining disease stage are detailed in Section 7.3.3 below.

### 7.3.1 Diagnostic Criteria

Various diagnostic criteria are available for diagnosing and/or predicting the onset of ALS. Exemplary diagnostic criteria for ALS include El Escorial criteria, Awaji Shima criteria, and Gold Coast criteria.

### 7.3.1.1 El Escorial Criteria

An ALS diagnosis may be carried out according to revised El Escorial criteria as described in Ludolph A et al., Amyotroph Lateral Scler Frontotemporal Degener. 2015; 16 (5-6): 291-2. According to such analysis, a subject's upper motor neuron (UMN) and lower motor neuron (LMN) impairment is assessed in bulbar, cervical, thoracic, and lumbosacral regions of the body. UMN assessment includes analysis of spasticity and hyper-reflexivity. LMN assessment includes assessment of weakness, atrophy, and fasciculation. Subjects with signs of UMN and LMN impairment in at least three regions tested are diagnosed as having “definite” ALS. Subjects with signs of UMN and LMN impairment in at least two regions tested are diagnosed as having “probable” ALS. Subjects with UMN and LMN signs in one region with evidence by electromyography (EMG) of LMN involvement in another region are characterized as having “laboratory supported probable” ALS. Subjects with signs of UMN and LMN impairment in one region tested or have UMN impairment alone in two or more regions are characterized as having “possible” ALS. Subjects with signs of LMN impairment alone in two or more regions tested are characterized as having “suspected” ALS.

### 7.3.1.2 Awaji Shima Criteria

ALS may be diagnosed according to Awaji shima criteria as described in deCarvalho M et al., Clin Neurophysiol. 2008; 119 (3): 497-503. Awaji shima criteria integrate electrophysiological criteria with clinical examination findings where the electrophysiological evidence is considered of equal weight to the clinical exam findings of lower motor neuron abnormality. In some examples, Awaji shima criteria and El Escorial criteria can be used in combination to diagnose a subject as having ALS. Electrophysiological criteria are assessed by EMG, a technique known in the art that detects electrical activity in muscles. EMG is useful in detecting the findings of acute denervation (fibrillation and positive sharp waves), chronic denervation (long-duration, complex motor unit action potentials [MUAP]), and chronic reinnervation (large amplitude MUAP).

To be indicative of ALS according to Awaji shima criteria, an EMG generally shows signs of acute or chronic denervation in at least three spinal levels (bulbar, cervical, thoracic, and/or lumbosacral). If three spinal levels are not abnormal, acute or chronic denervation needs to be evident in three extremities with the involvement of at least two muscles supplied by two different roots and two different nerves in each extremity. According to Awaji shima criteria, “clinically definite ALS” is defined by clinical or electrophysiological evidence by the presence of LMN as well as UMN signs in the bulbar region and at least 2 spinal regions or the presence of LMN and UMN signs in 3 spinal regions. “Clinically probable ALS” is defined by clinical or electrophysiological evidence of LMN and UMN signs in at least 2 regions with some UMN signs necessarily rostral to (above) the LMN signs. “Clinically possible ALS” is defined when clinical or electrophysiological signs of UMN and LMN dysfunction are found in only 1 region or UMN signs are found alone in 2 regions or LMN signs are found rostral to UMN signs.

### 7.3.1.3 Gold Coast Criteria

ALS may be diagnosed in a subject according to Gold Coast criteria as described in Shefner J M et al., Clin Neurophysiol 2020 August; 131 (8): 1975-1978. According to Gold Coast criteria, a subject may be diagnosed as having ALS if: 1) progressive motor impairment was documented by history or repeated clinical assessment, preceded by normal motor function; 2) upper and lower motor neuron dysfunction is present in at least one body region with either (i) upper and lower motor neuron dysfunction noted in the same body region if only one region is involved or (ii) lower motor neuron dysfunction in at least two body regions; and 3) other disease processes are excluded. Upper motor neuron dysfunction implies the presence of at least one of the following: increased deep tendon reflexes, including the presence of a reflex in a clinically weak and wasted muscle, or spread to adjacent muscles; or the presence of pathological reflexes, including Hoffman sign, Babinski sign, crossed adductor reflex, or snout reflex. Lower motor neuron dysfunction in a given muscle requires either: (1) clinical examination evidence of muscle weakness and muscle wasting; or (2) EMG abnormalities that generally include both (i) evidence of chronic neurogenic change, defined by large motor unit potentials of increased duration and/or increased amplitude (with polyphasia) and motor unit instability regarded as supportive but not obligatory evidence, and (ii) evidence of ongoing denervation, including fibrillation potentials or positive sharp waves, or fasciculation potentials.

### 7.3.2 Biomarkers

Various physiological parameters (e.g., levels of specified proteins or peptides, histological changes, levels of specific compounds in biological fluids, modulation in gene expression, etc.) can be used to monitor the course of treatment and to adjust the dosing of the active pharmaceutical ingredient (such as the antibodies described in Section 7.1.1) accordingly. In addition or alternatively, biomarkers can be used to select patients for treatment with the methods disclosed herein. In the following sections, various such biomarkers that can be used with the methods provided herein are described.

### 7.3.2.1 Biomarkers in Biological Samples

One or more specific substances in a biological sample of a patient, or the ratio of the level of one substance against another in the biological sample, the aggregate level of one kind of substance (e.g., total protein, gene expression) in the biological sample, or the ratio involving such aggregate level, can be used as biomarkers. Non-limiting examples of biological samples include CSF, serum, blood, peripheral blood mononuclear cells (PBMCs), urine, salvia, tissue, and the like. A biological sample can be collected from a patient using standard techniques known in the art. The substance that serves as the biomarker can be cells, tissue, protein, DNA, RNA, microRNA, lipid, metabolite or any other matter that can reflect the presence and/or progression of ALS. Methods of measuring biomarkers from a biological sample can include known methods, including but not limited to, ELISA, cytometric bead array analysis, immunonephelometric assays, genomic sequencing methods, real-time quantitative PCR, fluorescence activated cell sorting (FACS) analysis, chemical analysis, microscopic analysis, microarray, and the like. One of skill in the art would appreciate that the method of measuring the biomarker(s) in a biological sample depends on the type of sample and/or the biomarker of interest.

In certain embodiments, a biomarker suitable for use herein can be a substance (e.g., a protein, DNA, RNA) associated with neuroinflammation, systemic inflammation, mitochondrial dysfunction, neurodegeneration, protein aggregation, and or protein proteostasis. In some embodiments, a biomarker for ALS can be found in a biological fluid sample (e.g., blood, serum, PBMCs, CSF, urine). Some examples of such biomarkers for detection of ALS in are known in the art, e.g., Wilkins H M et al., AMYOTROPHIC LATERAL SCLEROSIS [Internet]. Brisbane (AU): Exon Publications; 2021 Jul. 25. Chapter 6 and Staats K A et al., Mol Neurodegener. 2022 Jan. 24; 17 (1): 11. In some embodiments, exemplary biomarkers include neurofilament light chain (NfL) protein in cerebrospinal fluid (CSF), serum, and/or blood. In some other embodiments, exemplary biomarkers include RNA and/or protein levels of adenosine deaminase (ADA) in cerebrospinal fluid (CSF), serum, and/or blood.

In some embodiments, a biomarker for ALS can be a genetic marker. Genetic markers for ALS can be found in the art, for example in the NHGRI-EBI GWAS Catalog (Sollis E et al., Nucleic Acids Res. 2022 Nov. 9: gkac1010). Non-limiting examples of genetic biomarkers for ALS include SOD1, C9orf72, TARDBP, FUS, ANG, VABP, SETX, DCTN1, VCP, UBQLN2, PFN1, and ATXN2. In certain embodiments, exemplary biomarkers include SOD1, C9orf72, TARDBP, and/or FUS.

### 7.3.2.2 Functional and Structural Biomarkers

As progression of ALS closely correlates with the functionality of the brain and nervous system, markers for neurological changes can also include functional markers. In some embodiments, cortical hyperexcitability can be a functional marker of ALS. Transcranial magnetic stimulation (TMS) can be used to assess motor cortical hyperexcitability as well as other neurophysiological exploration of the motor cortex in vivo. Other functional biomarkers of ALS can be assessed by other neurophysiological approaches, such as quantitative electroencephalography and the like.

One of skill in the art would appreciate that functional changes associated with ALS can also correlate with structural changes in the brain and/or other neurological tissues. In certain embodiments, diagnostic imaging including Magnetic Resonance Imaging (MRI) can be used to diagnosis ALS by observing such structural changes. Types of MRI scans than can be useful in diagnosis ALS include, but are not limited to structural MRI, voxel-based morphometry (VBM), diffusion tensor imaging (DTI), functional MRI (fMRI), and magnet resonance spectroscopy (MRS). Diagnostic imaging can also be used as a neurophysiological approach, for example to assess effective connectivity in different regions of the brain.

### 7.3.3 Disease Staging

Various staging systems are available for measuring the severity and/or rate of progression of ALS. Exemplary staging systems include the King's Clinical Staging system and the Milano-Torino Staging (MiToS) system. The MiToS system generally has a higher resolution in late disease, when functional involvement has developed, and the King's system generally has a higher resolution in early disease to mid-disease, when clinical or disease burden is increasing.

The King's system uses five stages, from 1 to 5 and is based on disease burden as measured by clinical involvement and significant feeding or respiratory failure, with stage 1 being symptom onset and stage 5 being death. ALS progression is defined by loss of independence in four key domains on the ALS Functional Rating Scale (ALSFRS): swallowing, walking/self-care, communicating and breathing. The King's system defines the stages as follows: stage 0, functional involvement but no loss of independence on any domain; stages 1-4, number of domains in which independence was lost; and stage 5, death.

The MiToS system involves six stages (0-5) based on functional ability assessed with the ALSFRS-R; stage 0 is defined as normal function and stage 5 is death. The MiToS stage at diagnosis correlates with survival at 18 months. Stage 1: symptom onset (involvement of first region); Stage 2A: diagnosis; Stage 2B: involvement of second region; Stage 3: involvement of third region; Stage 4A: need for gastrostomy; and Stage 4B: need for non-invasive ventilation.

### 7.4 Equivalents and Incorporation by Reference

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 8. ILLUSTRATIVE EMBODIMENTS

Illustrative embodiments of the present disclosure are provided in the paragraphs below:

1. A method of treating, preventing or ameliorating at least one symptom associated with amyotrophic lateral sclerosis (ALS) in a subject in need thereof, wherein the method comprises administering to the subject an effective amount of a recombinant anti-CD2 antibody or an antigen binding fragment thereof.

2. The method of embodiment 1, wherein the recombinant anti-CD2 antibody comprises:
(a) a heavy chain variable region CDR1 comprising the amino acid sequence of SEQ ID NO: 3;
(b) a heavy chain variable region CDR2 comprising the amino acid sequence of SEQ ID NO: 4;
(c) a heavy chain variable region CDR3 comprising the amino acid sequence of SEQ ID NO: 5;
(d) a light chain variable region CDR1 comprising the amino acid sequence of SEQ ID NO: 6;
(e) a light chain variable region CDR2 comprising the amino acid sequence of SEQ ID NO: 7; and
(f) a light chain variable region CDR3 comprising the amino acid sequence of SEQ ID NO: 8.

3. The method of embodiment 1 or embodiment 2, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 1.

4. The method of any one of embodiments 1-3, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 2.

5. The method of any one of embodiments 1-4, wherein the heavy chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 16.

6. The method of any one of embodiments 1-5, wherein the light chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 17.

7. The method of any one of embodiments 1-6, wherein the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 9, 10, 11, 12, or 13.

8. The method of any one of embodiments 1-7, wherein the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 14 or 15.

9. The method of embodiment 1, wherein the recombinant anti-CD2 antibody comprises:
(a) a heavy chain variable region CDR1 comprising the amino acid sequence of SEQ ID NO: 24;
(b) a heavy chain variable region CDR2 comprising the amino acid sequence of SEQ ID NO: 25;
(c) a heavy chain variable region CDR3 comprising the amino acid sequence of SEQ ID NO: 26;
(d) a light chain variable region CDR1 comprising the amino acid sequence of SEQ ID NO: 27;
(e) a light chain variable region CDR2 comprising the amino acid sequence of SEQ ID NO: 28; and
(f) a light chain variable region CDR3 comprising the amino acid sequence of SEQ ID NO: 29.

10. The method of embodiment 1 or embodiment 9, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 22.

11. The method of any one of embodiments 1, 9 or 10, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 23.

12. The method of any one of embodiments 1 or 9-11, wherein the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 30.

13. The method of any one of embodiments 1 or 9-11, wherein the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 31.

14. The method of embodiment 1, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 31, 32, 33, 34, or 35.

15. The method of embodiment 1 or embodiment 14, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 36, 37, 38, 39, or 40.

16. The method of any one of embodiments 1-15, wherein the anti-CD2 antibody or antigen binding fragment thereof comprises an additional CH2 domain.

17. The method of embodiment 16, wherein the additional CH2 domain comprises an amino acid sequence that is about or at least about 80%, 85%, 90%, 95%, 98%, or 99% identical to the amino acid sequence of SEQ ID NO: 18 or 19.

18. The method of any one of embodiments 1-17, wherein the anti-CD2 antibody or antigen binding fragment thereof comprises an additional CH3 domain.

19. The method of embodiment 18, wherein the additional CH3 domain comprises an amino acid sequence that is about or at least about 80%, 85%, 90%, 95%, 98%, or 99% identical to the amino acid sequence of SEQ ID NO: 20 or 21.

20. The method of any one of embodiments 1-19, wherein the anti-CD2 antibody or antigen binding fragment thereof is a humanized antibody.

21. The method of any one of embodiments 1-20, wherein the anti-CD2 antibody or antigen binding fragment thereof is an IgG1.

22. The method of any one of embodiments 1-20, wherein the anti-CD2 antibody or antigen binding fragment thereof is an IgG4.

23. The method of any one of embodiments 1-22, wherein the effective amount is sufficient to:
(a) increase the subject's Revised Amyotrophic Lateral Sclerosis Functional Rating Scale (ALSFRS-R) total score by at least 0.5 points;
(b) maintain the subject's ALSFRS-R total score; or
(c) slow the subject's disease progression rate by at least 0.5 ALSFRS-R/month, as compared to the ALSFRS-R total score obtained from the subject prior to the administering.

24. The method of any one of embodiments 1-23, wherein the effective amount is sufficient to increase life expectancy of the subject after the administering as compared to a subject having a similar diagnosis and rate of disease progression that was not administered the effective amount of a recombinant anti-CD2 antibody or an antigen binding fragment thereof.

25. The method of embodiment 24, wherein the increase is an increase of about or at least about a month, 3 months, 6 months, a year, 2 years, 3 years, 4 years, 5 years, or greater than 5 years.

26. The method of any one of embodiments 1-25, wherein the effective amount is sufficient to increase the percentage of the ratio of regulatory T cells to effector T cells (Treg:Teff) in a biological sample from the subject after the administering as compared to the percentage of Treg:Teff ratio in a biological sample obtained from the subject prior to the administering.

27. The method of embodiment 26, wherein the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

28. The method of any one of embodiments 1-27, wherein the effective amount is sufficient to decrease circulating T-cells in a biological sample from the subject after the administering as compared to the amount of circulating T-cells in a biological sample obtained from the subject prior to the administering.

29. The method of embodiment 28, wherein the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

30. The method of any one of embodiments 1-29, wherein the effective amount is sufficient to decrease natural killer (NK)-cells in a biological sample from the subject after the administering as compared to the amount of NK-cells in a biological sample obtained from the subject prior to the administering.

31. The method of embodiment 30, wherein the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

32. The method of any one of embodiments 1-31, wherein the effective amount is sufficient to increase regulatory T cells in a biological sample from the subject after the administering as compared to the amount of regulatory T cells in a biological sample obtained from the subject prior to the administering.

33. The method of embodiment 32, wherein the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

34. The method of any one of embodiments 26-33, wherein a biological sample comprises the subject's serum, peripheral blood mononuclear cells (PBMCs), cerebrospinal fluid (CSF), or any combination thereof.

35. The method of any one of embodiments 1-34, wherein subject in need thereof has been diagnosed as having familial ALS or sporadic ALS.

36. The method of any one of embodiments 1-35, wherein subject in need thereof has been diagnosed as having ALS within 36 months or less.

37. The method of any one of embodiments 1-35, wherein subject in need thereof has been diagnosed as having ALS within 24 months or less.

38. The method of any one of embodiments 1-37, wherein the at least one symptom associated with ALS comprises dyspnea, orthopnea, muscle weakness, dysphagia, dysarthria, or a combination of two or more thereof.

39. The method of any one of embodiments 1-38, wherein the administering to the subject comprises intravenous administration, intrathecal administration, or both.

40. The method of any one of embodiments 1-39, wherein the subject is currently being treated or has been previously treated with one or more ALS treatments.

41. The method of embodiment 40, wherein the one or more ALS treatments comprises riluzole, tauroursodeoxycholic acid (TUDCA), sodium phenylbutyrate, or any combination thereof.

42. The method of any one of embodiments 1-41, wherein the administering is for about or at least about 26 weeks.

43. The method of any one of embodiments 1-41, wherein the administering is for about or at least about 24 weeks.

44. The method of any one of embodiments 1-43, wherein the administering is once every week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 3 months, once every 3.5 months, once every 4 months, once every 4.5 months, once every 5 months, once every 5.5 months, or once every 6 months.

45. The method of any one of embodiments 1-44, wherein the effective amount is sufficient to achieve about 10 ng/ml recombinant anti-CD2 antibody or an antigen binding fragment thereof in the subject's CSF at a steady state.

46. The method of any one of embodiments 1-45, wherein the effective amount is sufficient to achieve a trough concentration (C0) greater than about 19 g/mL recombinant anti-CD2 antibody or an antigen binding fragment thereof in the subject. 47. The method of embodiment 45 or embodiment 46, wherein the effective amount is sufficient to achieve about 10 ng/ml recombinant anti-CD2 antibody or an antigen binding fragment thereof in the subject's CSF at a steady state for at least 30 days.

48. The method of embodiment 45 or embodiment 46, wherein the effective amount is sufficient to achieve about 10 ng/ml recombinant anti-CD2 antibody or an antigen binding fragment thereof in the subject's CSF at a steady state for at least 50 days.

49. The method of any one of embodiments 1-48, wherein the effective amount is from about 0.01 mg/kg to about 100 mg/kg and the administering is about every 4-14 weeks for a period of time sufficient to treat, prevent, or ameliorate at least one symptom associated with ALS in the subject.

50. The method of embodiment 49, wherein the administering occurs three times between the first administration and the last administration for a total of five administrations.

51. The method of embodiment 50, wherein the last administration is 26 weeks after the first administration.

52. The method of any one of embodiments 1-48, wherein the effective amount is from about 0.01 mg/kg to about 100 mg/kg and the administering is about every 4-16 weeks for a period of time sufficient to treat, prevent, or ameliorate at least one symptom associated with ALS in the subject.

53. The method of embodiment 52, wherein the administering occurs two times between the first administration and the last administration for a total of four administrations.

54. The method of embodiment 53, wherein the last administration is 24 weeks after the first administration.

### 9. EXAMPLES

Examples are provided below to illustrate the present invention. These examples are not meant to constrain the present invention to any particular application or theory of operation.

### 9.1 Example 1-A 52-Week, Safety, Pharmacokinetic and Pharmacodynamic Trial of an Anti-CD2 Antibody (e.g., Anti-CD2 Antibody 1) in Newly Diagnosed Adult Amyotrophic Lateral Sclerosis (ALS) Patients

The purpose of this study is to investigate the safety, pharmacokinetics and pharmacodynamics following administration of an anti-CD2 antibody (e.g., anti-CD2 Antibody 1) in newly diagnosed adult ALS patients. This study is directed to using an anti-CD2 mAb (e.g., anti-CD2 Antibody 1) to decrease pathogenic lymphocytes, such as circulating T-cells and NK-cells, while supporting regulatory T-cells (Treg) in order to reduce disease progression in ALS patients.

Objectives of the study include the following:
Evaluate the safety of anti-CD2 Antibody 1 in adult patients with ALS at 12 months;
Determine the pharmacokinetics and pharmacodynamics of anti-CD2 Antibody 1;
Correlate immunological profiles (e.g., Teff/Treg ratio) in blood and CSF with disease status;
Analyze biomarkers of Treg activity in serum, PBMCs, and/or CSF;
Determine time to tracheostomy/death;
Assess laboratory measurements and vital signs;
Assess change in ALSFRS-R over time; and
Analyze neurofilament light chain and ADA concentrations over time in serum and/or CSF.

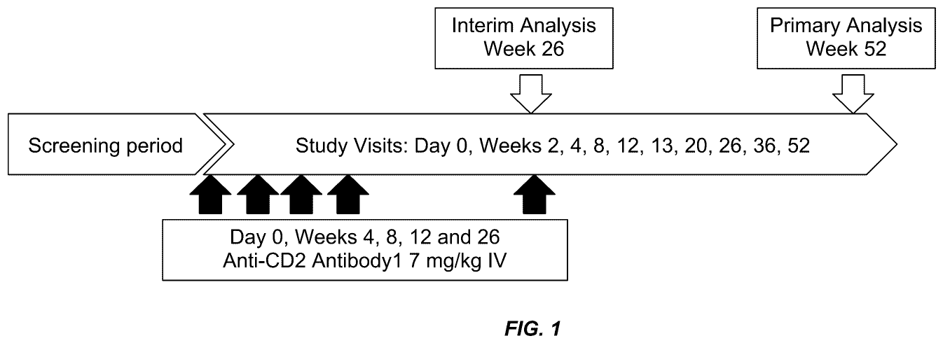

Study Design: Adults between 18-80 years of age recently diagnosed with sporadic ALS are recruited for this study. The duration of the study is 52 weeks following an initial screening period. After the screening period and following informed consent, subjects receive 5 doses of intravenous (IV) of an anti-CD2 mAb (e.g., anti-CD2 Antibody 1) at 7 mg/kg on Day 0 and Weeks 4, 8, 12 and 26 (FIG. 1). During and after completion of the anti-CD2 mAb (e.g., anti-CD2 Antibody 1) treatment period, subjects undergo follow-up visits at Weeks 2, 13, 20, 36 and 52. An interim analysis is performed at Week 26 and the primary analysis will be conducted at Week 52.

Treatment of ALS with best practice standard of care is permitted during the trial. However, subjects are maintained on a constant regimen (e.g., the same dose of riluzole and/or TUDCA, sodium phenylbutyrate, or TUDCA/sodium phenylbutyrate) from the screening period until the end of the study (EOS) at 12 months.

Population: Approximately 36 adult subjects with newly diagnosed sporadic ALS are enrolled. The sample size was chosen to provide sufficient patients to characterize the pharmacokinetic and mechanistic aspects of an anti-CD2 mAb (e.g., anti-CD2 Antibody 1) treatment in ALS. This sample size also provides a reasonable number of patients for an initial evaluation of safety in ALS. All subjects enrolled are followed for a minimum of 12 months.

### Inclusion Criteria:

1. Able to understand the study requirements and provide written informed consent before any study assessment is performed.
2. Subjects (male or female) aged 18 to 80 years.
3. Diagnosis of possible, laboratory supported probable, probable, or definite sporadic ALS by Revised El Escorial Criteria, study entry within 24-36 months of first symptoms.
4. Estimated ALS disease progression rate from first symptoms to Screening >0.5 ALSFRS-R/month.
5. Vital capacity greater than 60% of predicted for age, height, and sex at screening by slow vital or forced vital capacity (SVC or FVC).
6. Patients on existing ALS treatment with riluzole and/or TUDCA, sodium phenylbutyrate, or TUDCA/sodium phenylbutyrate must have been on a stable dose of for 28 days.
7. Up to date on vaccination requirements according to local guidelines.

### Exclusion Criteria:

1. Patient with severe systemic infections, current or within the two weeks prior to randomization.
2. Patient taking any other immunosuppression, except topical steroids.
3. Subjects with any other clinically significant medical or psychiatric condition, active infection or laboratory abnormality that would, in the judgment of the investigator, interfere with the subject's ability to participate in the study.
4. Subjects who, in the opinion of the investigator, are not capable of giving informed consent for the study or who are unable or unwilling to adhere to the study requirements outlined in the protocol.
5. Use of other investigational products or enrollment in another investigational drug study within 30 days of screening or 5 half-lives of the medication, whichever is longer.
6. Pregnant or nursing (lactating) women, where pregnancy is defined as the state of a female after conception and until the termination of gestation, confirmed by a positive human chorionic gonadotropin (hCG) laboratory test.
7. Women of child-bearing potential, defined as all women physiologically capable of becoming pregnant, unless they are using highly effective methods of contraception during dosing and for 24 weeks after the study medications have been stopped.

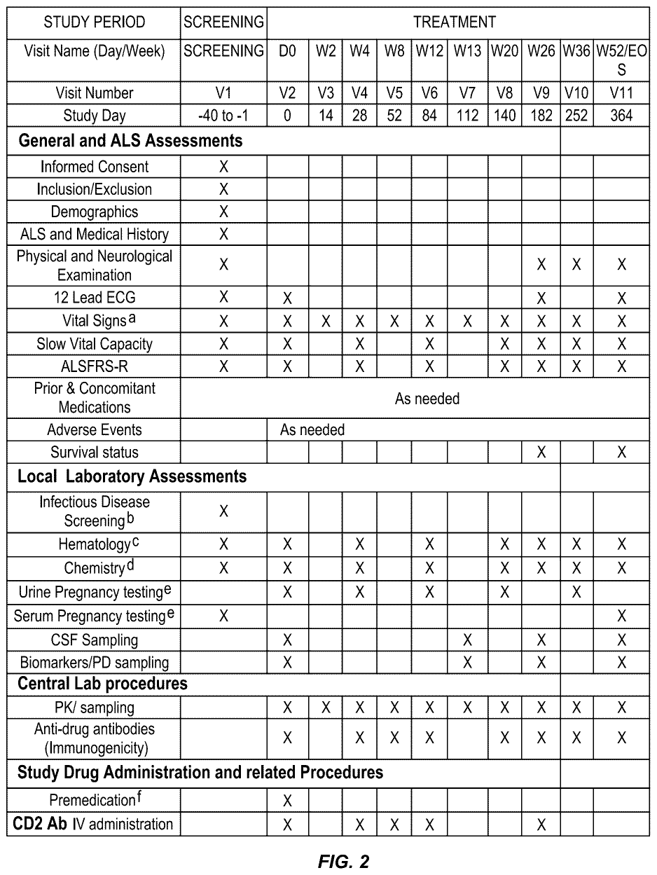

Study Assessments: During the course of the study, assessments are made according to the schedule of assessments provided in FIG. 2.

Where indicated in FIG. 2, safety assessments are performed at designated visits during the study. Safety assessments monitor for death, changes in ventilation, appearance of AEs and/or SAEs, and clinically significant changes in clinical chemistry, hematology, vital signs, and ECG. An adverse event (AE) is defined as any untoward medical occurrence in a subject administered a pharmaceutical product which does not necessarily have to have a causal relationship with this treatment. Serious adverse events (SAEs) are a subset of AEs and refer to any untoward medical occurrence that at any dose: 1) results in death; 2) is life threatening; 3) requires inpatient hospitalization or prolongation of existing hospitalization; and/or 4) results in persistent or significant disability/incapacity. Clinical chemistry includes measurement of a subject's sodium, potassium, chloride, urea, bicarbonate, creatinine, glucose, ALT, ALP, alkaline phosphatase, bilirubin, and CRP. Hematology includes measurement of a subject's hemoglobin, hematocrit, red blood cell (RBC), platelet count, white blood cells (WBC) with differential count (% and absolute). Vital signs include measuring a subject's blood pressure, pulse, body temperature, and respiratory rate. In addition, urine and serum pregnancy testing occur throughout the study for female participants of reproductive capacity.

Additional assessments are also performed at designated visits during the study as indicated in FIG. 2. A revised ALS Functional Rating Scale (ALSFRS-R) is administered by a healthcare provider during study visits to track changes in physical function, or lack thereof, during the study. Slow vital capacity (SVC), the volume of air expired through an unforced maneuver, is measured as an estimate respiratory function. Both ALSFRS-R and SVC are indicative of ALS disease status.

Blood and cerebrospinal fluid (CSF) samples are collected from the subjects. Serum is collected from the blood sample. Peripheral blood mononuclear cells (PBMCs) are isolated from the peripheral circulation (i.e., isolated from whole blood). PBMCs and CSF samples are subjected to lymphocyte profiling which measures the absolute number of T cells, B cells and NK cells in a subject. PBMCs and CSF samples are also subjected to a severe combined immunodeficiency (SCID) panel to determine B and T cell, T subsets, T-memory and naive subsets, and natural killer cell quantitation as determined by cell markers that include: CD3+; CD3+CD4+; CD3+CD8+; CD3-CD16/56+; CD19+; CD3+CD4+CD45RA+; CD3+CD4+CD45RO+; CD3+CD8+CD45RA+; CD3+CD8+CD45RO+; and a calculated CD4:CD8 ratio (T4:T8 ratio). In addition, PBMCs and CSF samples are subjected to flow cytometry FACS to measure cell subpopulations including CD2, CD3, CD4, CD5, CD8, CD19, CD25, CD27, CD38, CD45RA, CD45RO, CD59, CD127, CD138, CD154, FoxP3 and HLA-DR. Biomarkers of Treg activity are assessed in PBMCs, serum, and CSF samples. Biomarkers of Treg activity include 1) measuring amounts of immunosuppressive cytokines including TGF-beta, IL-10, and IL-35 in the samples, and/or 2) measuring amounts of Tregs present in the samples. Treg cells are identified as CD3+CD4+CD25+FoxP3+ cells having additional cell surface markers CD39, 5′Nucleotidase/CD73, CTLA-4, GITR, LAG-3, LRRC32, and Neuropilin-1. Neurofilament light chain and adenosine deaminase (ADA) concentrations are measured in serum and CSF samples. The amount of anti-CD2 antibody (e.g., anti-CD2 Antibody 1) is measured in serum and CSF samples.

Data Analysis: All information obtained on(S) AEs is displayed by subject. The number and percentage of subjects with(S) AEs are tabulated by body system and preferred term. A subject with multiple(S) AEs within a body system is only counted once towards the total of this body system. No formal statistical analysis is done for the safety and efficacy evaluation.

Pharmacodynamic and lymphocyte profiling data is analyzed in relation to disease status and response to gain mechanistic insights on effects of an anti-CD2 antibody (e.g., anti-CD2 Antibody 1) in this population. The magnitude and duration of pharmacodynamic effect of an anti-CD2 antibody (e.g., anti-CD2 Antibody 1) is characterized by measuring the change in peripheral immunophenotype, including all subsets of T-, B- and NK-cells. Summary statistics are also provided such as mean, median, SD, minimum and maximum, by visit/time for all PD. Summary statistics are produced for ALSFRS-R and SVC. These parameters are compared to historical control data from the PRO-ACT or other ALS datasets.

The immunomodulatory approach described herein using an anti-CD2 antibody (e.g., anti-CD2 Antibody 1) (five 7 mg/kg doses on Day 0, Weeks 4, 8, 12 and 26) decreases pathogenic lymphocytes by ~97% of circulating T-cells and 90% of NK-cells, while supporting regulatory T-cells (Treg) and may be able to reduce ALS disease progression as determined, in part, by ALSFRS-R and SVC scores.

Study Stopping Rules: The study will be placed on hold pending review by the Data Monitoring Committee (DMC) when any participant develops: 1) a Grade 3 infection or other infectious event per CTCAE criteria; and/or 2) an infection or other AE<Grade 2 that the investigator judges to be significant.

### 9.2 Example 2-A 52-Week, Safety, Pharmacokinetic and Pharmacodynamic Trial of an Anti-CD2 Antibody (e.g., Anti-CD2 Antibody 1) in Newly Diagnosed Adult Amyotrophic Lateral Sclerosis (ALS) Patients

The purpose of this study is to investigate the safety, pharmacokinetics, and pharmacodynamics of four 10 mg/kg doses of an anti-CD2 antibody (e.g., anti-CD2 Antibody 1) in newly diagnosed adult ALS patients. This study uses an anti-CD2 antibody (e.g., anti-CD2 Antibody 1) at four 10 mg/kg doses on Day 0 (baseline), Weeks 4, 8 and 24. The dose regimen was selected for providing anti-CD2 antibody (e.g., anti-CD2 Antibody 1) concentrations within the CNS and periphery sufficient to achieve both T cell depletion and immunomodulation.

Objectives of the study include the following:
Evaluate the safety and tolerability of an anti-CD2 antibody (e.g., anti-CD2 Antibody 1) in adult patients with ALS over 12 months;
Evaluate pharmacokinetics (PK) and pharmacodynamics (PD) of an anti-CD2 antibody (e.g., anti-CD2 Antibody 1) in blood and CSF;
Correlate leucocyte phenotypic profiles in blood and CSF with disease status;
Evaluate the time to tracheostomy/death;
Assess clinically relevant changes in laboratory measurements and vital signs;
Explore the mean ALSFRS-R and change in ALSFRS-R over time;
Measure SVC and change in SVC over time; and
Analyze ALS biomarkers, including neurofilament light chain (NfL), in serum and CSF.

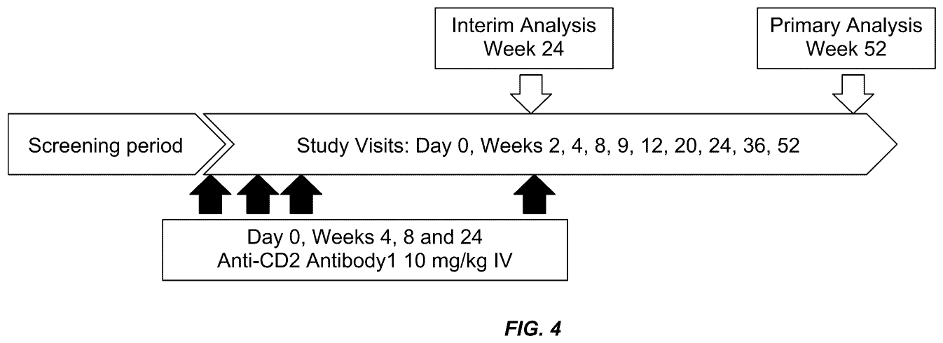

Study Design: Approximately 48 patients with sporadic ALS are enrolled as subjects for the study and followed for 12 months. Following informed consent, the eligible subjects intravenously (IV) receive 4 doses of an anti-CD2 antibody (e.g., anti-CD2 Antibody 1) (10 mg/kg) on Day 0 and Weeks 4, 8 and 24 (See FIG. 4). During and after completion of the anti-CD2 mAb (e.g., anti-CD2 Antibody 1) treatment period, subjects undergo follow-up visits at Weeks 2, 4, 8, 9, 12, 20, 24, 36 and 52. An interim analysis is performed at Week 24 and the primary analysis will be conducted at Week 52.

Treatment of ALS with a best practice standard of care is permitted during the study. Concomitant approved ALS treatment (e.g., riluzole) is allowed for all subjects; however, patients are maintained on a constant regimen (e.g., the same dose of riluzole) from screening until the end of the study (EOS) at 12 months.

Population: The sample size of approximately 48 patients with sporadic ALS is chosen to provide sufficient patients to characterize the pharmacokinetic and mechanistic aspects of an anti-CD2 mAb (e.g., anti-CD2 Antibody 1) treatment in ALS. This sample size also provides a reasonable number of patients for an initial evaluation of safety in ALS.

Inclusion Criteria: The eligible subjects in this study fulfill all the following criteria:
1. Able to understand the study requirements and provide written informed consent before any study assessment is performed;
2. Male or female patients ≥18 to 80 years of age;
3. Diagnosis of possible, laboratory supported probable, probable, or definite sporadic ALS by revised El Escorial Criteria (See, e.g., 7.3.1.1), at study entry within 24 months of first symptoms;
4. Estimated ALS disease progression rate from first symptoms to Screening ≥0.5 ALSFRS-R units/month (See, e.g., paragraph [00164]);
5. Vital capacity ≥60% of predicted for age, height, and sex at screening by slow vital capacity (SVC) (See, e.g., paragraph [00166]);
6. Patients on existing ALS treatment with riluzole must have been on a stable dose for 28 days; and
7. Up to date on age specific vaccinations according to local guidelines, including seasonal influenza and SARS-COV-2/COVID-19 as well as Hepatitis B and Pneumococcal disease.

Exclusion Criteria: Patients meeting any of the following criteria are not eligible for inclusion in this study:
1. Patients with severe systemic infections, current or within the two weeks prior to randomization;
2. History of recent [within 3 Months of Screening (Visit 1)] or ongoing serious uncontrolled bacterial, viral, fungal, or other opportunistic infections, including:
   Human immunodeficiency virus (HIV)
   Current or prior infection with hepatitis B (HBV), as indicated by positive HBsAg or positive HBcAb
   Current or prior hepatitis C (HCV), unless treated with anti-viral therapy with achievement of a sustained virologic response (undetectable viral load (VL) 12 weeks after cessation of therapy)
   Positive Interferon Gamma Release Assay (IGRA) TB test, e.g., Quantiferon-TB Gold or Quantiferon-TB Gold Plus tests
   Active infection with EBV as defined by EBV viral load ≥10,000 copies per 10⁶ PBMCs or ≥2,000 copies per mL of whole blood
   Active infection with Cytomegalovirus (CMV) as defined by CMV viral load ≥10,000 IU or copies per mL of whole blood or plasma;
3. Subjects with any other clinically significant medical or psychiatric condition, active infection or laboratory abnormality that would interfere with the subject's ability to participate in the study;
4. Subjects with any of the following: hemoglobin (Hbg)<8 mg/dL, WBC count≤1,500/mm³, ALC<800 cells/mL or platelet count≤75,000/mm³;
5. Subjects who are not capable of giving informed consent for the study or who are unable or unwilling to adhere to the study requirements outlined in the protocol;
6. Use of other investigational products or treatment in another investigational drug study within 30 days of screening or 5 half-lives of the medication, whichever is longer;
7. Pregnant or nursing (lactating) women, where pregnancy is defined as the state of a female after conception and until the termination of gestation, confirmed by a positive human chorionic gonadotropin (hCG) laboratory test; and
8. Women of child-bearing potential, defined as all women physiologically capable of becoming pregnant, unless they are using highly effective methods of contraception during dosing and for 24 weeks after the study medications have been stopped.

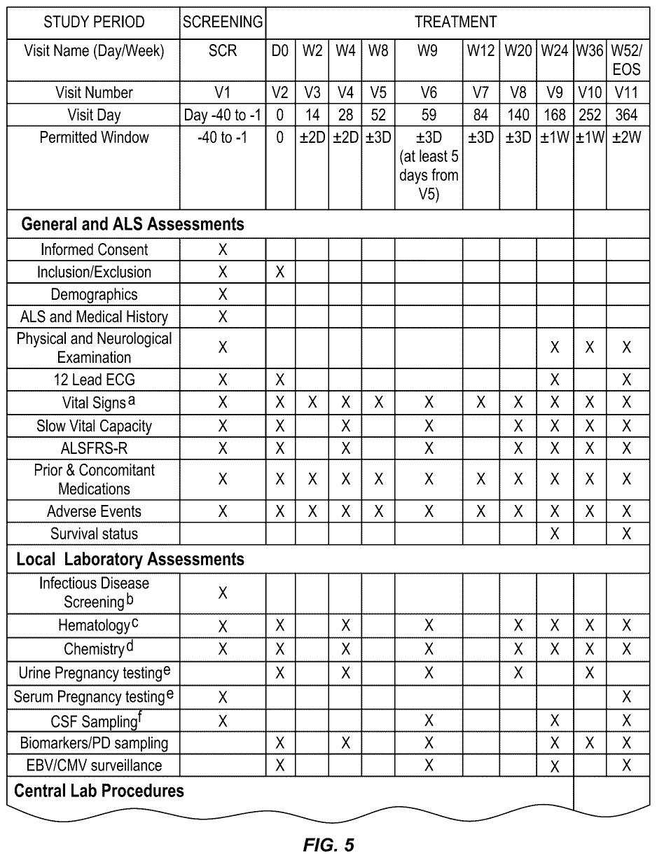

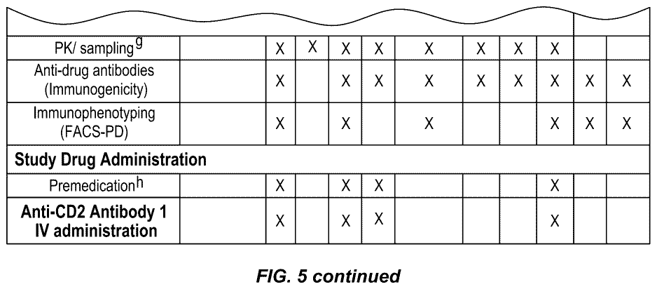

Study Assessments: During the course of the study, assessments are made according to the schedule of assessments provided in FIG. 5.

Where indicated in FIG. 5, safety assessments are performed at designated visits during the study. Safety assessments monitor for death, changes in ventilation, appearance of AEs and/or SAEs, and clinically significant changes in clinical chemistry, hematology, vital signs, and ECG. An adverse event (AE) is defined as any untoward medical occurrence in a subject administered a pharmaceutical product which does not necessarily have to have a causal relationship with this treatment. Serious adverse events (SAEs) are a subset of AEs and refer to any untoward medical occurrence that at any dose: 1) results in death; 2) is life threatening; 3) requires inpatient hospitalization or prolongation of existing hospitalization; and/or 4) results in persistent or significant disability/incapacity. Clinical chemistry includes measurement of a subject's sodium, potassium, chloride, urea, bicarbonate, creatinine, glucose, ALT, ALP, alkaline phosphatase, bilirubin, and CRP. Hematology includes measurement of a subject's hemoglobin, hematocrit, red blood cell (RBC), platelet count, white blood cells (WBC) with differential count (% and absolute). Vital signs include measuring a subject's blood pressure, pulse, body temperature, and respiratory rate. In addition, urine and serum pregnancy testing occur throughout the study for female participants of reproductive capacity.

Additional assessments are also performed at designated visits during the study as indicated in FIG. 5. A revised ALS Functional Rating Scale (ALSFRS-R) is administered by a healthcare provider during study visits to track changes in physical function, or lack thereof, during the study. Slow vital capacity (SVC), the volume of air expired through an unforced maneuver, is measured as an estimate respiratory function. Both ALSFRS-R and SVC are indicative of ALS disease status.

Blood and cerebrospinal fluid (CSF) samples are collected from the subjects. Serum is collected from the blood sample. Peripheral blood mononuclear cells (PBMCs) are isolated from the peripheral circulation (i.e., isolated from whole blood). Blood and CSF samples are tested for ALS biomarkers. For example, serum neurofilament light chain (Nfl) is increased due to neuronal damage in ALS and therefore is known in the art as a diagnostic and prognostic biomarker for ALS and ALS disease progression. In the present study, NfL in serum and CSF is measured by a specific ELISA and examined for correlation to disease status in the subjects. The change of NfL in serum and CSF over time and is examined for correlations with ALSFRS-R score and/or survival.

PBMCs and CSF samples are subjected to lymphocyte profiling which measures the absolute number of T cells, B cells and NK cells in a subject. PBMCs and CSF samples are also subjected to flow cytometry (FACS) to evaluate leukocyte phenotypes over the duration of the study according to methods described in Section 9.1). The functional status of all leukocyte populations in blood, PBMCs and CSF is assessed using single cell RNA-sequencing (scRNA-seq) using the 10× Genomics platform, and is measured and presented as change over time. Peripheral blood mononuclear cells (PBMCs) are isolated using density gradient centrifugation. CSF samples with visible blood contamination or >200 RBCs/μl are excluded from scRNA-seq analysis. Correlations between leukocyte composition and ALSFRS-R score and/or survival is determined. B-cell receptor and T-cell receptor composition, as determined by scRNAseq, are presented as change over time and correlations between clonality and ALSFRS-R score or survival is determined. Changes in cellular differentiation, analyzed through pseudotime inference, are used to order the cells along a trajectory—a path—that describes how clonally expanded T cells traverse through different states. Alterations in cell signaling and cellular phenotypes are determined by gene set enrichment analysis. Exploratory transcriptomic analysis (e.g., Serial and Cap analysis of gene expression (SAGE/CAGE), RNA sequencing, RNA microarray) can be performed where needed/appropriate.

The amount of anti-CD2 antibody (e.g., anti-CD2 Antibody 1) is measured in serum and CSF samples collected from study subjects. Anti-CD2 antibody concentrations are determined by a validated ELISA method with a Lower Limit of Quantification (LLOQ) of 10 ng/mL. Concentrations are expressed in mass per volume units. Anti-CD2 antibody plasma concentration data is listed by subject, and visit/sampling time point. Descriptive summary statistics are provided by treatment and visit/sampling time point, including the frequency (n, %) of concentrations below LLOQ and reported as zero. Summary statistics includes mean (arithmetic and geometric), SD (standard deviation), coefficient of variation (CV) (arithmetic and geometric), median, minimum and maximum. Concentrations below LLOQ are treated as zero in summary statistics and for PK parameter calculations. A geometric mean is not reported if the dataset includes zero values.

Based on the expected PK/PD activity peripheral anti-CD2 antibody (e.g., anti-CD2 Antibody 1) trough (C0) concentrations of >19 g/mL results in adequate target engagement and lymphocyte suppression in the CNS, lymphoid and peripheral vascular compartments. To maintain a trough concentration (C0)>19 g/mL over a 30-day dosing interval, assuming standard clearance and an 8.5-day half-life, IV administration of 10 mg/kg anti-CD2 antibody (e.g., anti-CD2 Antibody 1) is administered. To provide adequate exposure with minimal accumulation, subjects are treated with three (3) IV anti-CD2 antibody (e.g., anti-CD2 Antibody 1) doses over 2 months. Following the dose at month 2 (week 8), the anti-CD2 antibody (e.g., anti-CD2 Antibody 1) concentrations drop below the limit of quantification (10 ng/mL) 50 days thereafter resulting in approximately 90 days of PK exposure in the CNS and 110 days systemically.

Data Analysis: Pharmacodynamic and leukocyte profiling/leukocyte composition data are analyzed in relation to disease status and response to assess mechanistic effects of anti-CD2 antibody (e.g., anti-CD2 Antibody 1) in the ALS population. The magnitude and duration of pharmacodynamic effect of an anti-CD2 antibody (e.g., anti-CD2 Antibody 1) is characterized by measuring the change in peripheral immunophenotype and functional immune status (scRNA-seq), including all subsets of T-, B- and NK-cells. Summary statistics are produced for ALSFRS-R and SVC. These parameters are compared to historical control data from the PRO-ACT or other ALS datasets.

The following pharmacokinetic (PK) parameters of the anti-CD2 antibody (e.g., anti-CD2 Antibody 1) are determined using the actual recorded sampling times and non-compartmental method(s) from the plasma concentration-time data: Cₘₐₓ, Tₘₐₓ, area under the curve (AUC) last, the area from time of dosing extrapolated to infinity (AUC_{inf}), T_{1/2}, volume of distribution based on the terminal elimination phase following extravascular (Vz/F) administration, and clearance following extravascular (CL/F) administration. The linear trapezoidal rule is used for (AUC) calculation. Regression analysis of the terminal plasma elimination phase for the determination of T_{1/2} includes at least 3 data points after Cₘₐₓ. If the adjusted R² value of the regression analysis of the terminal phase is less than 0.75, no values are reported for T_{1/2}, AUC_{inf} and CL.

All information obtained on(S) AEs is displayed by subject. The number and percentage of subjects with(S) AEs is tabulated by body system and preferred term. A subject with multiple(S) AEs within a body system is only counted once towards the total of this body system. No formal statistical analysis is done for the safety and efficacy evaluation.

The immunomodulatory approach described herein using an anti-CD2 antibody (e.g., anti-CD2 Antibody 1) at four 10 mg/kg doses on Day 0, Weeks 4, 8 and 24 decreases pathogenic lymphocytes by ~97% of circulating T-cells and 90% of NK-cells, while supporting regulatory T-cells (Treg) and may be able to reduce ALS disease progression as determined, in part, by ALSFRS-R and SVC scores.

Study Stopping Rules: The study is placed on hold if any participant develops 1) a single Grade 4 or higher infection per CTCAE criteria, and/or 2) three or more infections or other clinically significant events >Grade 3 considered to be anti-CD2 antibody related.

### SEQUENCES

| SEQ ID | | |
|---|---|---|
| NO | DESCRIPTION | SEQUENCE |
| 1 | heavy chain | QVQLVQSGAEVQRPGASVKVSCKASGYIFTEYYMYWVRQA |
| | variable region | PGQGLELVGRIDPEDGSIDYVEKFKKKVTLTADTSSSTAY |
| | | MELSSLTSDDTAVYYCARGKENYRFAYWGQGTLVTVSS |
| 2 | light chain | DVVMTQSPPSLLVTLGQPASISCRSSQSLLHSSGNTYLNW |
| | variable region | LLQRPGQSPQPLIYLVSKLESGVPDRFSGSGSGTDFTLKI |
| | | SGVEAEDVGVYYCMQFTHYPYTFGQGTKLEIK |
| 3 | heavy chain | EYYMY |
| | variable region | |
| | CDR1 | |
| 4 | heavy chain | RIDPEDGSIDYVEKFKK |
| | variable region | |
| | CDR2 | |
| 5 | heavy chain | GKFNYRFAY |
| | variable region | |
| | CDR3 | |
| 6 | light chain | RSSQSLLHSSGNTYLN |
| | variable region | |
| | CDR1 | |
| 7 | light chain | LVSKLES |
| | variable region | |
| | CDR2 | |
| 8 | light chain | MQFTHYPYT |
| | variable region | |
| | CDR3 | |
| 9 | heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQT |
| | | YICNVNHKPSNTKVDKRVEPKSCDKTHTCPPCPAPELLGG |
| | | PSVFLFPPKPKDTLMISRTPEVTCVVVDVSHEDPEVKENW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 10 | heavy chain | ASTKGPSVFPLAPSSKSTSGGTAALGCLVKDYFPEPVTVS |
| | constant region | WNSGALTSGVHTFPAVLQSSGLHSLSSVVTVPSSSLGTQT |
| | | YICNVNHKPSNTKVDKKVEPKSCDKTHTCPPCPAPELLGG |
| | | PSVELFPPKPKDTLMISRTPEVTCVVVDVSHEDPEVKENW |
| | | YVDGVEVHNAKTKPREEQYNSTYRVVSVLTVLHQDWLNGK |
| | | EYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 11 | heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKENW |
| | | YVDGVEVHNAKTKPREEQYNSTYRVVSVLTVLHQDWLNGK |
| | | EYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNIFSCSVMHEALHNHYT |
| | | QKSLSLSPGK |
| 12 | heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKENW |
| | | YVDGVEVHNAKTKPREEQYNSTYRVVSVLTVLHQDWLNGK |
| | | EYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVESCSVMHEALHNHYT |
| | | QKSLSLSPGK |
| 13 | heavy chain | ASTKGPSVFPLAPSSKSTSGGTAALGCLVKDYFPEPVTVS |
| | constant region | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVELFPPKPKDTLMISRTPEVTCVVVDVSHEDPEVKENW |
| | | YVDGVEVHNAKTKPREEQYNSTYRVVSVLTVLHQDWLSGK |
| | | EYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 14 | light chain | RTVAAPSVFIFPPSDEQLKSGTASVVCLLNNFYPREAKVQ |
| | constant region | WKVDNALQSGNSQESVTEQDSKDSTYSLSSTLTLSKADYE |
| | | KHKLYACEVTHQGLSSPVTKSENRGEC |
| 15 | light chain | RTVAAPSVFIFPPSDEQLKSGTASVVCLLNNFYPREAKVQ |
| | constant region | WKVDNALQSGNSQESVTEQDSKDSTYSLSSTLTLSKADYE |
| | | KHKVYACEVTHQGLSSPVTKSENRGEC |
| 16 | optimized heavy | CAAGTGCAGCTGGTGCAGAGCGGAGCTGAGGTGCAGAGAC |
| | chain sequence | CCGGCGCCAGCGTCAAGGTGAGCTGTAAGGCCAGCGGCTA |
| | | CATCTTCACAGAATACTACATGTACTGGGTGAGGCAAGCC |
| | | CCCGGCCAAGGACTGGAGCTGGTGGGCAGAATCGATCCAG |
| | | AGGATGGCAGCATCGACTACGTGGAGAAGTTCAAGAAGAA |
| | | GGTGACTCTGACAGCCGACACAAGCAGCAGCACTGCTTAC |
| | | ATGGAGCTGAGCTCTCTGACTAGCGATGACACTGCCGTGT |
| | | ACTACTGTGCTAGGGGCAAGTTCAACTATAGGTTCGCCTA |
| | | CTGGGGCCAAGGCACTCTGGTGACAGTCAGCAGCGCTAGC |
| | | ACCAAGGGCCCATCGGTCTTCCCCCTGGCACCCTCCTCCA |
| | | AGAGCACCTCTGGGGGCACAGCGGCCCTGGGCTGCCTGGT |
| | | CAAGGACTACTTCCCCGAACCGGTGACGGTGTCGTGGAAC |
| | | TCAGGCGCCCTGACCAGCGGCGTGCACACCTTCCCGGCCG |
| | | TCCTACAGTCCTCAGGACTCTACTCCCTCAGCAGCGTGGT |
| | | GACCGTGCCCTCCAGCAGCTTGGGCACCCAGACCTACATC |
| | | TGCAACGTGAATCACAAGCCCAGCAACACCAAGGTGGACA |
| | | AGAAGGTTGAGCCCAAATCTTGTGACAAAACTCACACATG |
| | | CCCACCGTGCCCAGCACCTGAACTCCTGGGGGGACCGTCA |
| | | GTCTTCCTCTTCCCCCCAAAACCCAAGGACACCCTCATGA |
| | | TCTCCCGGACCCCTGAGGTCACATGCGTGGTGGTGGACGT |
| | | GAGCCACGAAGACCCTGAGGTCAAGTTCAACTGGTACGTG |
| | | GACGGCGTGGAGGTGCATAATGCCAAGACAAAGCCGCGGG |
| | | AGGAGCAGTACAACAGCACGTACCGTGTGGTCAGCGTCCT |
| | | CACCGTCCTGCACCAGGACTGGCTGAATGGCAAGGAGTAC |
| | | AAGTGCAAGGTCTCCAACAAAGCCCTCCCAGCCCCCATCG |
| | | AGAAAACCATCTCCAAAGCCAAAGGGCAGCCCCGAGAACC |
| | | ACAGGTGTACACCCTGCCCCCATCCCGGGACGAGCTGACC |
| | | AAGAACCAGGTCAGCCTGACCTGCCTGGTCAAAGGCTTCT |
| | | ATCCCAGCGACATCGCCGTGGAGTGGGAGAGCAATGGGCA |
| | | GCCGGAGAACAACTACAAGACCACGCCTCCCGTGCTGGAC |
| | | TCCGACGGCTCCTTCTTCCTCTACAGCAAGCTCACCGTGG |
| | | ACAAGAGCAGGTGGCAGCAGGGGAACGTCTTCTCATGCTC |
| | | CGTGATGCATGAGGCTCTGCACAACCACTACACGCAGAAG |
| | | AGCCTCTCCCTGTCTCCGGGTAAATGATGA |
| 17 | optimized Light | GACGTGGTGATGACTCAGAGCCCTCCTTCTCTGCTGGTGA |
| | chain sequence | CTCTGGGCCAGCCAGCCAGCATCAGCTGTAGGAGCAGCCA |
| | | GTCTCTGCTGCACTCCAGCGGCAACACTTATCTGAACTGG |
| | | CTGCTGCAGAGACCCGGCCAGAGCCCTCAGCCTCTGATCT |
| | | ACCTCGTGAGCAAGCTGGAGAGCGGCGTGCCAGATAGGTT |
| | | TAGCGGCAGCGGAAGCGGCACTGACTTCACTCTGAAGATC |
| | | AGCGGCGTGGAAGCTGAGGATGTGGGCGTCTACTACTGCA |
| | | TGCAGTTCACACACTACCCATACACTTTCGGCCAAGGCAC |
| | | AAAGCTGGAAATCAAGCGTACGGTGGCTGCACCATCTGTC |
| | | TTCATCTTCCCGCCATCTGATGAGCAGTTGAAATCTGGAA |
| | | CTGCCTCTGTTGTGTGCCTGCTGAATAACTTCTATCCCAG |
| | | AGAGGCCAAAGTACAGTGGAAGGTGGATAACGCCCTCCAA |
| | | TCGGGTAACTCCCAGGAGAGTGTCACAGAGCAGGACAGCA |
| | | AGGACAGCACCTACAGCCTCAGCAGCACCCTGACGCTGAG |
| | | CAAAGCAGACTACGAGAAACACAAAGTCTACGCCTGCGAA |
| | | GTCACCCATCAGGGCCTGAGCTCGCCCGTCACAAAGAGCT |
| | | TCAACAGGGGAGAGTGTTGATGA |
| 18 | CH2 domain | SVFLFPPKPKDTLMISRTPEVTCVVVDVSAEDPEVQENWY |
| | | VDGVEVHNAKTKPREEQFNSTFRVVSVLTVLHQDWLNGKE |
| | | YKCKVSNKGLPSSIEKTISKTK |
| 19 | CH2 domain | SVFLFPPKPKDTLMISRTPEVTCVVVDVSQEDPEVQENWY |
| | | VDGVEVHNAKTKPREEQFNSTYRVVSVLTVLHQDWLNGKE |
| | | YKCKVSNKGLGSSIEKTISKAK |
| 20 | CH3 domain | GQPREPQVYTLPPSREEMTKNQVSLTCLVKGFYPSDIAVE |
| | | WESNGQPENNYKTTPPMLDSDGSFFLYSKLTVDKSRWQQG |
| | | NVFSCSVMHEALHNHYTQKSLSLSPGK |
| 21 | CH3 domain | GQPREPQVYTLPPSQEEMTKNQVSLTCLVKGFYPSDIAVE |
| | | WESNGQPENNYKTTPPVLDSDGSFFLYSRLTVDKSRWQEG |
| | | NVFSCSVMHEALHNHYTQKSLSLSLGK |
| 22 | heavy chain | EVQLVESGGGLVQPGGSLRLSCAASGFTESDYWMSWVRQA |
| | variable region | PGKRLEWIGHIKYDGSYTNYAPSLKNRFTISRDNAKNSLY |
| | | LQMNSLRSEDTAVYYCAREAPGAASYWGQGTLVTVSS |
| 23 | light chain | DVVLTQTPLSLSVTPGQPASISCRSSQSLVHSNGNTYLEW |
| | variable region | FLQKPGQSPQLLIYKVSNRFSGVPDRESGSGSGSDETLKI |
| | | SRVEAEDVGVYYCFQGTHDPYTFGQGTKVEIK |
| 24 | heavy chain | DYWMS |
| | variable region | |
| | CDR1 | |
| 25 | heavy chain | HIKYDGSYTNYAPSLKNRF |
| | variable region | |
| | CDR2 | |
| 26 | heavy chain | EAPGAASY |
| | variable region | |
| | CDR3 | |
| 27 | light chain | RSSQSLVHSNGNTYLE |
| | variable region | |
| | CDR1 | |
| 28 | light chain | KVSNRES |
| | variable region | |
| | CDR2 | |
| 29 | light chain | FQGTHDPYT |
| | variable region | |
| | CDR3 | |
| 30 | heavy chain | ASTKGPSVFPLAPSSRSTSESTAALGCLVKDYFPEPVTVS |
| | constant region | WNSGSLTSGVHTFPAVLQSSGLYSLSSVVTVPSSSLGTQT |
| | | YVCNVNHKPSNTKVDKRVEIKTCGGGSKPPTCPPCPAPEL |
| | | LGGPSVFLFPPKPKDTLMISRTPEVTCVVVDVSQEDPDVK |
| | | FNWYVNGAEVHHAQTKPRETQYNSTYRVVSVLTVTHQDWL |
| | | NGKEYTCKVSNKALPAPIQKTISKDKGQPREPQVYTLPPS |
| | | REELTKNQVSLTCLVKGFYPSDIVVEWESSGQPENTYKTT |
| | | PPVLDSDGSYFLYSKLTVDKSRWQQGNVFSCSVMHEALHN |
| | | HYTQKSLSVSPGK |
| 31 | light chain | RAVAAPSVEIFPPSEDQVKSGTVSVVCLLNNFYPREASVK |
| | constant region | WKVDGVLKTGNSQESVTEQDSKDNTYSLSSTLTLSNTDYQ |
| | | SHNVYACEVTHQGLSSPVTKSENRGEC |
| 32 | light chain | DIVMTQSPLSLSVTPGEPASISCRSSQSLVHSNGNTYLEW |
| | variable region | FLQKPGQSPQLLIYKVSNRFSGVPDRESGSGSGTDFTLKI |
| | | SRVEAEDVGVYYCFQGTHDPYTFGQGTKLEIK |
| 33 | light chain | DIVMTQSPLSLPVTPGEPASISCRSSQSLVHSNGNTYLEW |
| | variable region | FLQKPGQSPQLLIYKVSNRFSGVPDRESGSGSGTDETLKI |
| | | SRVEAEDVGVYYCFQGTHDPYTFGQGTKLEIK |
| 34 | light chain | DVVLTQSPLSLPVTPGEPASISCRSSQSLVHSNGNTYLEW |
| | variable region | FLQKPGQSPQLLIYKVSNRFSGVPDRESGSGSGTDFTLKI |
| | | SRVEAEDVGVYYCFQGTHDPYTFGQGTKLEIK |
| 35 | light chain | DIVMTQSPLSLPVTPGEPASISCRSSQSLLHSNGNNYLEW |
| | variable region | FLQKPGQSPQLLIYKGSNRASGVPDRESGSGSGTDFTLKI |
| | | SRVEAEDVGVYYCFQGTHDPYTFGQGTKLEIK |
| 36 | heavy chain | EVQLVESGGGLVQPGGSLRLSCAASGFTFSDYWMSWVRQA |
| | variable region | PGKRLEWIGHIKYDGSYTNYVPSVKNRFTISRDNAKNSLY |
| | | LQMNSLRSEDTAVYYCAREAPGAASYWGQGTLVTVSS |
| 37 | heavy chain | EVQLVESGGGLVQPGGSLRLSCAASGFTESDYWMSWVRQA |
| | variable region | PGKRLEWIGHIKYDGSYTNYVDSVKNRFTISRDNAKNSLY |
| | | LQMNSLRAEDTAVYYCAREAPGAASYWGQGTLVTVSS |
| 38 | heavy chain | EVQLVESGGGLVQPGGSLRLSCAASGFTFSDYWMSWVRQA |
| | variable region | PGKGLEWIGHIKYDGSYTNYVDSVKGRFTISRDNAKNSLY |
| | | LQMNSLRAEDTAVYYCAREAPGAASYWGQGTLVTVSS |
| 39 | heavy chain | EVQLVESGGGLVQPGGSLRLSCAASGFTESDYWMSWVRQA |
| | variable region | PGKGLEWIGHIKYDGSYTNYVPSVKGRFTISRDNAKNSLY |
| | | LQMNSLRAEDTAVYYCAREAPGAASYWGQGTLVTVSS |
| 40 | heavy chain | EVQLVESGGGLVQPGGSLRLSCAASGFTESDYWMSWVRQA |
| | variable region | PGKGLEWVGHIKYDGSYKNYVDSVKGRFTISRDNAKNSLY |
| | | LQMNSLRAEDTAVYYCAREAPGAASYWGQGTLVTVSS |

What is claimed is:

1. A method of treating, preventing or ameliorating at least one symptom associated with amyotrophic lateral sclerosis (ALS) in a subject in need thereof, wherein the method comprises administering to the subject an effective amount of a recombinant anti-CD2 antibody or an antigen binding fragment thereof.

2. The method of claim 1, wherein the recombinant anti-CD2 antibody comprises:
(a) a heavy chain variable region CDR1 comprising the amino acid sequence of SEQ ID NO: 3;
(b) a heavy chain variable region CDR2 comprising the amino acid sequence of SEQ ID NO: 4;
(c) a heavy chain variable region CDR3 comprising the amino acid sequence of SEQ ID NO: 5;
(d) a light chain variable region CDR1 comprising the amino acid sequence of SEQ ID NO: 6;
(e) a light chain variable region CDR2 comprising the amino acid sequence of SEQ ID NO: 7; and
(f) a light chain variable region CDR3 comprising the amino acid sequence of SEQ ID NO: 8.

3. The method of claim 1 or claim 2, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 1.

4. The method of any one of claims 1-3, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 2.

5. The method of any one of claims 1-4, wherein the heavy chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 16.

6. The method of any one of claims 1-5, wherein the light chain of the anti-CD2 antibody or antigen binding fragment thereof is encoded by a nucleotide sequence comprising about or at least about 80%, 85%, 90%, 91%, 92%, 93%, 94%, 95%, 96%, 97%, 98%, 99%, or 100% identity to SEQ ID NO: 17.

7. The method of any one of claims 1-6, wherein the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 9, 10, 11, 12, or 13.

8. The method of any one of claims 1-7, wherein the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 14 or 15.

9. The method of claim 1, wherein the recombinant anti-CD2 antibody comprises:
(a) a heavy chain variable region CDR1 comprising the amino acid sequence of SEQ ID NO: 24;
(b) a heavy chain variable region CDR2 comprising the amino acid sequence of SEQ ID NO: 25;
(c) a heavy chain variable region CDR3 comprising the amino acid sequence of SEQ ID NO: 26;
(d) a light chain variable region CDR1 comprising the amino acid sequence of SEQ ID NO: 27;
(e) a light chain variable region CDR2 comprising the amino acid sequence of SEQ ID NO: 28; and
(f) a light chain variable region CDR3 comprising the amino acid sequence of SEQ ID NO: 29.

10. The method of claim 1 or claim 9, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 22.

11. The method of any one of claim 1, 9 or 10, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 23.

12. The method of any one of claim 1 or 9-11, wherein the recombinant anti-CD2 antibody or an antigen binding fragment thereof comprises a heavy chain constant region comprising an amino acid sequence that is about or at least about 80%, 85%, 90%, 91%, 92%, 93%, 94%, 95%, 96%, 97%, 98%, 99%, or 100% identical to SEQ ID NO: 30.

13. The method of any one of claim 1 or 9-11, wherein the recombinant anti-CD2 antibody or an antigen binding fragment thereof comprises a light chain constant region comprising an amino acid sequence that is about or at least about 80%, 85%, 90%, 91%, 92%, 93%, 94%, 95%, 96%, 97%, 98%, 99%, or 100% identical to SEQ ID NO: 31.

14. The method of claim 1, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 31, 32, 33, 34, or 35.

15. The method of claim 1 or claim 14, wherein the anti-CD2 antibody or antigen binding fragment thereof comprises an amino acid sequence that is about or at least about 80%, 85%, 90%, 91%, 92%, 93%, 94%, 95%, 96%, 97%, 98%, 99%, or 100% identical to SEQ ID NO: 36, 37, 38, 39, or 40.

16. The method of any one of claims 1-15, wherein the anti-CD2 antibody or antigen binding fragment thereof comprises an additional CH2 domain.

17. The method of claim 16, wherein the additional CH2 domain comprises an amino acid sequence that is about or at least about 80%, 85%, 90%, 95%, 98%, or 99% identical to the amino acid sequence of SEQ ID NO: 18 or 19.

18. The method of any one of claims 1-17, wherein the anti-CD2 antibody or antigen binding fragment thereof comprises an additional CH3 domain.

19. The method of claim 18, wherein the additional CH3 domain comprises an amino acid sequence that is about or at least about 80%, 85%, 90%, 95%, 98%, or 99% identical to the amino acid sequence of SEQ ID NO: 20 or 21.

20. The method of any one of claims 1-19, wherein the anti-CD2 antibody or antigen binding fragment thereof is a humanized antibody.

21. The method of any one of claims 1-20, wherein the anti-CD2 antibody or antigen binding fragment thereof is an IgG1.

22. The method of any one of claims 1-20, wherein the anti-CD2 antibody or antigen binding fragment thereof is an IgG4.

23. The method of any one of claims 1-22, wherein the effective amount is sufficient to:
(a) increase the subject's Revised Amyotrophic Lateral Sclerosis Functional Rating Scale (ALSFRS-R) total score by at least 0.5 points;
(b) maintain the subject's ALSFRS-R total score; or
(c) slow the subject's disease progression rate by at least 0.5 ALSFRS-R/month, as compared to the ALSFRS-R total score obtained from the subject prior to the administering.

24. The method of any one of claims 1-23, wherein the effective amount is sufficient to increase life expectancy of the subject after the administering as compared to a subject having a similar diagnosis and rate of disease progression that was not administered the effective amount of a recombinant anti-CD2 antibody or an antigen binding fragment thereof.

25. The method of claim 24, wherein the increase is an increase of about or at least about a month, 3 months, 6 months, a year, 2 years, 3 years, 4 years, 5 years, or greater than 5 years.

26. The method of any one of claims 1-25, wherein the effective amount is sufficient to increase the percentage of the ratio of regulatory T cells to effector T cells (Treg:Teff) in a biological sample from the subject after the administering as compared to the percentage of Treg:Teff ratio in a biological sample obtained from the subject prior to the administering.

27. The method of claim 26, wherein the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

28. The method of any one of claims 1-27, wherein the effective amount is sufficient to decrease circulating T-cells in a biological sample from the subject after the administering as compared to the amount of circulating T-cells in a biological sample obtained from the subject prior to the administering.

29. The method of claim 28, wherein the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

30. The method of any one of claims 1-29, wherein the effective amount is sufficient to decrease natural killer (NK)-cells in a biological sample from the subject after the administering as compared to the amount of NK-cells in a biological sample obtained from the subject prior to the administering.

31. The method of claim 30, wherein the decrease is a decrease of about or at least about 5%, 10%, 15%, 20%, 25%, 30%, 35%, 40%, 45%, 50%, 55%, 60%, 65%, 70%, 75%, 80%, 85%, 90%, 95%, or 100%.

32. The method of any one of claims 1-31, wherein the effective amount is sufficient to increase regulatory T cells in a biological sample from the subject after the administering as compared to the amount of regulatory T cells in a biological sample obtained from the subject prior to the administering.

33. The method of claim 32, wherein the increase is an increase of about or at least about 5%, 10%, 15%, 20%, 25%, 30%, 35%, 40%, 45%, 50%, 55%, 60%, 65%, 70%, 75%, 80%, 85%, 90%, 95%, 100%, or greater than 100%.

34. The method of any one of claims 26-33, wherein a biological sample comprises the subject's serum, peripheral blood mononuclear cells (PBMCs), cerebrospinal fluid (CSF), or any combination thereof.

35. The method of any one of claims 1-34, wherein subject in need thereof has been diagnosed as having familial ALS or sporadic ALS.

36. The method of any one of claims 1-35, wherein subject in need thereof has been diagnosed as having ALS within 36 months or less.

37. The method of any one of claims 1-35, wherein subject in need thereof has been diagnosed as having ALS within 24 months or less.

38. The method of any one of claims 1-37, wherein the at least one symptom associated with ALS comprises dyspnea, orthopnea, muscle weakness, dysphagia, dysarthria, or a combination of two or more thereof.

39. The method of any one of claims 1-38, wherein the administering to the subject comprises intravenous administration, intrathecal administration, or both.

40. The method of any one of claims 1-39, wherein the subject is currently being treated or has been previously treated with one or more ALS treatments.

41. The method of claim 40, wherein the one or more ALS treatments comprises riluzole, tauroursodeoxycholic acid (TUDCA), sodium phenylbutyrate, or any combination thereof.

42. The method of any one of claims 1-41, wherein the administering is for about or at least about 26 weeks.

43. The method of any one of claims 1-41, wherein the administering is for about or at least about 24 weeks.

44. The method of any one of claims 1-43, wherein the administering is once every week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 3 months, once every 3.5 months, once every 4 months, once every 4.5 months, once every 5 months, once every 5.5 months, or once every 6 months.

45. The method of any one of claims 1-44, wherein the effective amount is sufficient to achieve about 10 ng/ml recombinant anti-CD2 antibody or an antigen binding fragment thereof in the subject's CSF at a steady state.

46. The method of any one of claims 1-45, wherein the effective amount is sufficient to achieve a trough concentration (C0) greater than about 19 g/mL recombinant anti-CD2 antibody or an antigen binding fragment thereof in the subject.

47. The method of claim 45 or claim 46, wherein the effective amount is sufficient to achieve about 10 ng/ml recombinant anti-CD2 antibody or an antigen binding fragment thereof in the subject's CSF at a steady state for at least 30 days.

48. The method of claim 45 or claim 46, wherein the effective amount is sufficient to achieve about 10 ng/ml recombinant anti-CD2 antibody or an antigen binding fragment thereof in the subject's CSF at a steady state for at least 50 days.

49. The method of any one of claims 1-48, wherein the effective amount is from about 0.01 mg/kg to about 100 mg/kg and the administering is about every 4-14 weeks for a period of time sufficient to treat, prevent, or ameliorate at least one symptom associated with ALS in the subject.

50. The method of claim 49, wherein the administering occurs three times between the first administration and the last administration for a total of five administrations.

51. The method of claim 50, wherein the last administration is 26 weeks after the first administration.

52. The method of any one of claims 1-48, wherein the effective amount is from about 0.01 mg/kg to about 100 mg/kg and the administering is about every 4-16 weeks for a period of time sufficient to treat, prevent, or ameliorate at least one symptom associated with ALS in the subject.

53. The method of claim 52, wherein the administering occurs two times between the first administration and the last administration for a total of four administrations.

54. The method of claim 53, wherein the last administration is 24 weeks after the first administration.


SEQUENCE LISTING (40 sequences)

SEQ ID NO: 1 - protein, 118 aa, synthetic construct
  source 1..118: note = heavy chain variable region
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 2 - protein, 112 aa, synthetic construct
  source 1..112: note = light chain variable region
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 3 - protein, 5 aa, synthetic construct
  source 1..5: note = heavy chain variable region CDR1
EYYMY

SEQ ID NO: 4 - protein, 17 aa, synthetic construct
  source 1..17: note = heavy chain variable region CDR2
RIDPEDGSIDYVEKFKK

SEQ ID NO: 5 - protein, 9 aa, synthetic construct
  source 1..9: note = heavy chain variable region CDR3
GKFNYRFAY

SEQ ID NO: 6 - protein, 16 aa, synthetic construct
  source 1..16: note = light chain variable region CDR1
RSSQSLLHSSGNTYLN

SEQ ID NO: 7 - protein, 7 aa, synthetic construct
  source 1..7: note = light chain variable region CDR2
LVSKLES

SEQ ID NO: 8 - protein, 9 aa, synthetic construct
  source 1..9: note = light chain variable region CDR3
MQFTHYPYT

SEQ ID NO: 9 - protein, 330 aa, synthetic construct
  source 1..330: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 330 aa, synthetic construct
  source 1..330: note = heavy chain constant region
ASTKGPSVFPLAPSSKSTSGGTAALGCLVKDYFPEPVTVSWNSGALTSGVHTFPAVLQSSGLHSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 330 aa, synthetic construct
  source 1..330: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 12 - protein, 330 aa, synthetic construct [sequence omitted]

SEQ ID NO: 13 - protein, 330 aa, synthetic construct
  source 1..330: note = heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLSGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct
  source 1..107: note = light chain constant region
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 15 - protein, 107 aa, synthetic construct [sequence omitted]

SEQ ID NO: 16 - DNA, 1350 bp, synthetic construct
  source 1..1350: mol_type = other DNA; note = optimized heavy chain sequence
caagtgcagctggtgcagagcggagctgaggtgcagagacccggcgccagcgtcaaggtgagctgtaaggccagcggctacatcttcacagaatactacatgtactgggtgaggcaagcccccggccaaggactggagctggtgggcagaatcgatccagaggatggcagcatcgactacgtggagaagttcaagaagaaggtgactctgacagccgacacaagcagcagcactgcttacatggagctgagctctctgactagcgatgacactgccgtgtactactgtgctaggggcaagttcaactataggttcgcctactggggccaaggcactctggtgacagtcagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatga

SEQ ID NO: 17 - DNA, 663 bp, synthetic construct
  source 1..663: mol_type = other DNA; note = optimized Light chain sequence
gacgtggtgatgactcagagccctccttctctgctggtgactctgggccagccagccagcatcagctgtaggagcagccagtctctgctgcactccagcggcaacacttatctgaactggctgctgcagagacccggccagagccctcagcctctgatctacctcgtgagcaagctggagagcggcgtgccagataggtttagcggcagcggaagcggcactgacttcactctgaagatcagcggcgtggaagctgaggatgtgggcgtctactactgcatgcagttcacacactacccatacactttcggccaaggcacaaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatga

SEQ ID NO: 18 - protein, 102 aa, synthetic construct
  source 1..102: note = CH2 domain
SVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTK

SEQ ID NO: 19 - protein, 102 aa, synthetic construct
  source 1..102: note = CH2 domain
SVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAK

SEQ ID NO: 20 - protein, 107 aa, synthetic construct
  source 1..107: note = CH3 domain
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 107 aa, synthetic construct
  source 1..107: note = CH3 domain
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 117 aa, synthetic construct
  source 1..117: note = heavy chain variable region
EVQLVESGGGLVQPGGSLRLSCAASGFTFSDYWMSWVRQAPGKRLEWIGHIKYDGSYTNYAPSLKNRFTISRDNAKNSLYLQMNSLRSEDTAVYYCAREAPGAASYWGQGTLVTVSS

SEQ ID NO: 23 - protein, 112 aa, synthetic construct
  source 1..112: note = light chain variable region
DVVLTQTPLSLSVTPGQPASISCRSSQSLVHSNGNTYLEWFLQKPGQSPQLLIYKVSNRFSGVPDRFSGSGSGSDFTLKISRVEAEDVGVYYCFQGTHDPYTFGQGTKVEIK

SEQ ID NO: 24 - protein, 5 aa, synthetic construct
  source 1..5: note = heavy chain variable region CDR1
DYWMS

SEQ ID NO: 25 - protein, 19 aa, synthetic construct
  source 1..19: note = heavy chain variable region CDR2
HIKYDGSYTNYAPSLKNRF

SEQ ID NO: 26 - protein, 8 aa, synthetic construct
  source 1..8: note = heavy chain variable region CDR3
EAPGAASY

SEQ ID NO: 27 - protein, 16 aa, synthetic construct
  source 1..16: note = light chain variable region CDR1
RSSQSLVHSNGNTYLE

SEQ ID NO: 28 - protein, 7 aa, synthetic construct
  source 1..7: note = light chain variable region CDR2
KVSNRFS

SEQ ID NO: 29 - protein, 9 aa, synthetic construct
  source 1..9: note = light chain variable region CDR3
FQGTHDPYT

SEQ ID NO: 30 - protein, 333 aa, synthetic construct
  source 1..333: note = heavy chain constant region
ASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 31 - protein, 107 aa, synthetic construct
  source 1..107: note = light chain constant region
RAVAAPSVFIFPPSEDQVKSGTVSVVCLLNNFYPREASVKWKVDGVLKTGNSQESVTEQDSKDNTYSLSSTLTLSNTDYQSHNVYACEVTHQGLSSPVTKSFNRGEC

SEQ ID NO: 32 - protein, 112 aa, synthetic construct
  source 1..112: note = light chain variable region
DIVMTQSPLSLSVTPGEPASISCRSSQSLVHSNGNTYLEWFLQKPGQSPQLLIYKVSNRFSGVPDRFSGSGSGTDFTLKISRVEAEDVGVYYCFQGTHDPYTFGQGTKLEIK

SEQ ID NO: 33 - protein, 112 aa, synthetic construct
  source 1..112: note = light chain variable region
DIVMTQSPLSLPVTPGEPASISCRSSQSLVHSNGNTYLEWFLQKPGQSPQLLIYKVSNRFSGVPDRFSGSGSGTDFTLKISRVEAEDVGVYYCFQGTHDPYTFGQGTKLEIK

SEQ ID NO: 34 - protein, 112 aa, synthetic construct
  source 1..112: note = light chain variable region
DVVLTQSPLSLPVTPGEPASISCRSSQSLVHSNGNTYLEWFLQKPGQSPQLLIYKVSNRFSGVPDRFSGSGSGTDFTLKISRVEAEDVGVYYCFQGTHDPYTFGQGTKLEIK

SEQ ID NO: 35 - protein, 112 aa, synthetic construct
  source 1..112: note = light chain variable region
DIVMTQSPLSLPVTPGEPASISCRSSQSLLHSNGNNYLEWFLQKPGQSPQLLIYKGSNRASGVPDRFSGSGSGTDFTLKISRVEAEDVGVYYCFQGTHDPYTFGQGTKLEIK

SEQ ID NO: 36 - protein, 117 aa, synthetic construct
  source 1..117: note = heavy chain variable region
EVQLVESGGGLVQPGGSLRLSCAASGFTFSDYWMSWVRQAPGKRLEWIGHIKYDGSYTNYVPSVKNRFTISRDNAKNSLYLQMNSLRSEDTAVYYCAREAPGAASYWGQGTLVTVSS

SEQ ID NO: 37 - protein, 117 aa, synthetic construct
  source 1..117: note = heavy chain variable region
EVQLVESGGGLVQPGGSLRLSCAASGFTFSDYWMSWVRQAPGKRLEWIGHIKYDGSYTNYVDSVKNRFTISRDNAKNSLYLQMNSLRAEDTAVYYCAREAPGAASYWGQGTLVTVSS

SEQ ID NO: 38 - protein, 117 aa, synthetic construct
  source 1..117: note = heavy chain variable region
EVQLVESGGGLVQPGGSLRLSCAASGFTFSDYWMSWVRQAPGKGLEWIGHIKYDGSYTNYVDSVKGRFTISRDNAKNSLYLQMNSLRAEDTAVYYCAREAPGAASYWGQGTLVTVSS

SEQ ID NO: 39 - protein, 117 aa, synthetic construct
  source 1..117: note = heavy chain variable region
EVQLVESGGGLVQPGGSLRLSCAASGFTFSDYWMSWVRQAPGKGLEWIGHIKYDGSYTNYVPSVKGRFTISRDNAKNSLYLQMNSLRAEDTAVYYCAREAPGAASYWGQGTLVTVSS

SEQ ID NO: 40 - protein, 117 aa, synthetic construct
  source 1..117: note = heavy chain variable region
EVQLVESGGGLVQPGGSLRLSCAASGFTFSDYWMSWVRQAPGKGLEWVGHIKYDGSYKNYVDSVKGRFTISRDNAKNSLYLQMNSLRAEDTAVYYCAREAPGAASYWGQGTLVTVSS